(19) United States
(12) Patent Application Publication
(10) Pub. No.: US 2020/0395103 A1
(43) Pub. Date: Dec. 17, 2020

(54) METHODS OF PERFORMING CLINICAL TRIALS

(71) Applicant: KLARITOS, INC., Menlo Park, CA (US)

(72) Inventors: Vijay RAMAKRISHNAN, Palo Alto, CA (US); Edwin P. CHING, Woodside, CA (US)

(21) Appl. No.: 16/971,500

(22) PCT Filed: Feb. 20, 2019
(86) PCT No.: PCT/US2019/018811

§ 371 Date: Aug. 20, 2020

Related U.S. Application Data
(60) Provisional application No. 62/633,583, filed on Feb. 21, 2018.
(60) Provisional application No. 62/646,634, filed on Mar. 22, 2018.
(60) Provisional application No. 62/651,644, filed on Apr. 2, 2018.
(60) Provisional application No. 62/729,157, filed on Sep. 10, 2018.
(60) Provisional application No. 62/768,616, filed on Nov. 16, 2018.
(60) Provisional application No. 62/776,339, filed on Dec. 6, 2018.
(60) Provisional application No. 62/792,382, filed on Jan. 14, 2019.
(60) Provisional application No. 62/792,444, filed on Jan. 15, 2019.

(51) Int. Cl. G16H 10/20; G06F 16/31

(52) U.S. Cl. CPC G16H 10/20; G06F 16/31

(57) ABSTRACT

Disclosed herein are methods of performing clinical trials. Also disclosed herein are methods of obtaining payments for a medicament. Also disclosed herein are systems and devices for selecting a medicament for a subject.

### CROSS-REFERENCE

This application claims the benefit of U.S. Provisional Application No. 62/633,583, filed Feb. 21, 2018; U.S. Provisional Application No. 62/646,634, filed Mar. 22, 2018; U.S. Provisional Application No. 62/651,644, filed Apr. 2, 2018; U.S. Provisional Application No. 62/729,157, filed Sep. 10, 2018; U.S. Provisional Application No. 62/768,616, filed Nov. 16, 2018; U.S. Provisional Application No. 62/776,339, filed Dec. 6, 2018; U.S. Provisional Application No. 62/792,382, filed Jan. 14, 2019; and U.S. Provisional Application No. 62/792,444, filed Jan. 15, 2019; each of which is incorporated by reference herein in their entirety.

### SUMMARY

Disclosed herein are methods that can comprise administering a medicament to a subject enrolled in a clinical trial, where: a clinical trial provider can be paid by a third-party payer only if the medicament shows efficacy in the subject, for so long as the medicament shows efficacy in the subject, on a subject-by-subject basis, where at least one subject does not show efficacy, and the third-party payer can be a party who does not at least partially own or have licensed intellectual property to the medicament, its formulation, or its method of use. In some embodiments, the clinical trial may not be conducted by a manufacturer of the medicament. In some embodiments, the third-party payer may not be an entity that directly paid a manufacturer of the medicament. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, a diagnostic test may not be approved or cleared by a regulatory agency. In some embodiments, a regulatory agency can be the US Food and Drug Administration (FDA). In some embodiments, a diagnostic test can comprise next generation sequencing. In some embodiments, a diagnostic test can comprise accessing a database. In some embodiments, a database can be a relational database. In some embodiments, a diagnostic test can comprise machine learning. In some embodiments, from about 1 to about 8,000 subjects may not show efficacy when a medicament is administered. In some embodiments, from about 1% to about 50% or subjects may not show efficacy when a medicament is administered.

Also disclosed herein are methods that can comprise administering a non-approved and non-licensed medicament to a subject in a therapeutically effective amount in a clinical trial, where the medicament clinical trial provider can be reimbursed for the medicament based on efficacy on a subject-by-subject basis, and where the reimbursement may not be provided by the manufacturer of the medicament or the subject. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, a diagnostic test may not be approved or cleared by a regulatory agency. In some embodiments, a regulatory agency can be the US Food and Drug Administration (FDA). In some embodiments, a diagnostic test can comprise next generation sequencing. In some embodiments, a diagnostic test can comprise accessing a database. In some embodiments, a database can be a relational database. In some embodiments, a diagnostic test can comprise machine learning. In some embodiments, from about 1 to about 8,000 subjects may not show efficacy when a medicament is administered. In some embodiments, from about 1% to about 50% or subjects may not show efficacy when a medicament is administered.

Also disclosed herein are methods that can comprise administering a medicament to a subject in a clinical trial, where the medicament was approved or licensed for a first condition, where the administering can be effective to at least partially treat a second condition that may not be the first condition, and wherein the medicament clinical trial provider can be reimbursed for the medicament based on efficacy on a subject-by-subject basis. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, a diagnostic test may not be approved or cleared by a regulatory agency. In some embodiments, a regulatory agency can be the US Food and Drug Administration (FDA). In some embodiments, a diagnostic test can comprise next generation sequencing. In some embodiments, a diagnostic test can comprise accessing a database. In some embodiments, a database can be a relational database. In some embodiments, a diagnostic test can comprise machine learning. In some embodiments, from about 1 to about 8,000 subjects may not show efficacy when a medicament is administered. In some embodiments, from about 1% to about 50% or subjects may not show efficacy when a medicament is administered.

Also disclosed herein are methods that can comprise administering a medicament to a subject in a clinical trial, where data from the clinical trial can be periodically entered into a clinical trial database, the subject can be administered the medicament and stays on the medicament in the clinical trial as long as the medicament shows efficacy, and the clinical trial provider can be paid for efficacy, on a subject-by-subject basis by a payer, wherein the third-party payer may not be the subject or can be a party who does not at least partially own or have licensed intellectual property to the medicament, its formulation, or its method of use. In some embodiments, the financial and/or legal liabilities related to the clinical trial may rest with the third-party payer. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, a diagnostic test may not be approved or cleared by a regulatory agency. In some embodiments, a regulatory agency can be the US Food and Drug Administration (FDA). In some embodiments, a diagnostic test can comprise next generation sequencing. In some embodiments, a diagnostic test can comprise accessing a database. In some embodiments, a database can be a relational database. In some embodiments, a diagnostic test can comprise machine learning. In some embodiments, from about 1 to about 8,000 subjects may not show efficacy when a medicament is administered. In some embodiments, from about 1% to about 50% or subjects may not show efficacy when a medicament is administered.

Also disclosed herein are methods of stratifying a subject pool of subjects having or suspected of having a disease or condition that can comprise: performing an assay on samples from subjects in the subject pool, or obtaining data derived therefrom, to determine a disease state genotype or immunological feature; and stratifying the subject pool, wherein the stratifying can be performed based on the assay of data derived therefrom and comprises: enrolling subjects to receive a medicament, wherein the enrolling comprises admission to a series of clinical trials conducted simultaneously, or admission to a multi-arm clinical trial conducted simultaneously, wherein a third-party sponsor of the series of clinical trials and the multi-arm clinical trial are the same, and wherein the medicament may not be licensed or approved by a regulatory agency; and providing conventional treatment that can be a licensed and approved drug, wherein the subjects do not participate in the clinical trial; wherein the medicament and the conventional treatment treat the same condition. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, a diagnostic test may not be approved or cleared by a regulatory agency. In some embodiments, a regulatory agency can be the US Food and Drug Administration (FDA). In some embodiments, a diagnostic test can comprise next generation sequencing. In some embodiments, a diagnostic test can comprise accessing a database. In some embodiments, a database can be a relational database. In some embodiments, a diagnostic test can comprise machine learning. In some embodiments, from about 1 to about 8,000 subjects may not show efficacy when a medicament is administered. In some embodiments, from about 1% to about 50% or subjects may not show efficacy when a medicament is administered.

Also disclosed herein are methods of stratifying a subject pool of subjects having or suspected of having a disease or condition that can comprise: performing an assay on samples from subjects in the subject pool, or obtaining data derived therefrom, to determine a disease state genotype or immunological feature; and stratifying the subject pool, wherein the stratifying can be performed based on the assay of data derived therefrom and comprises: enrolling subjects to receive a medicament, wherein the enrolling comprises admission to a series of clinical trials conducted simultaneously, or admission to a multi-arm clinical trial conducted simultaneously, wherein a third-party sponsor of the series of clinical trials and the multi-arm clinical trial are the same, and wherein the medicament can be licensed and approved but may not be marketed in the territory that the medicament is licensed and approved in; and providing conventional treatment, wherein the conventional treatment can be a licensed and approved drug that can be marketed in the territory that the conventional treatment is licensed and approved in, and wherein the subjects do not participate in the clinical trial; wherein the medicament and the conventional treatment treat the same condition. In some embodiments, the medicament may not be marketed because of a freedom to operate issue raised from one or more claims from one or more patents in the territory in which the medicament is licensed and approved. In some embodiments, the medicament is supplied from a pharmacy. In some embodiments, the third-party sponsor is a party who does not at least partially own or have licensed intellectual property to the medicament, its formulation, or its method of use. In some embodiments, the third-party sponsor is responsible for designing, managing, and/or executing the clinical trial; the trial data developed by the third-party sponsor are owned by the sponsor. In some embodiments, a sponsor is the sole representative to communicate with FDA for the clinical trial matters. In some embodiments, the financial and/or legal liabilities related to the clinical trial rest with the third-party sponsor. In some embodiments, the third-party sponsor may only be charged if the medicament shows efficacy in the subject, for so long as the medicament shows efficacy in the subject. In some embodiments, the third-party sponsor may only be charged on a subject-by-subject basis. In some embodiments, the third-party sponsor can be charged based on the percentage of subjects that the medicament shows efficacy in. In some embodiments, the stratifying further comprises providing a prior authorization for subjects to enroll in the series of clinical trials or the multi-arm clinical trial. In some embodiments, the series of clinical trials or the multi-arm clinical trial are performed for discovery of a biomarker of the disease or condition. In some embodiments, the series of clinical trials or the multi-arm clinical trial are performed for discovery of a diagnostic for the disease or condition. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, an in vitro diagnostic test may not be approved or cleared by a regulatory agency. In some embodiments, from about 1 to about 8,000 subjects may not show efficacy when a medicament is administered. In some embodiments, from about 1% to about 50% or subjects may not show efficacy when a medicament is administered.

Also disclosed herein are methods that can comprise administering a medicament to a subject enrolled in a clinical trial, wherein at least 4 clinical trials are conducted in parallel, and wherein: each of the at least 4 clinical trials employs a medicament, the medicament in each of the at least 4 clinical trials can be different, each medicament can be targeted to the same disease or condition, at least one medicament can be a non-licensed and non-approved medicament, one third-party sponsor sponsors all of the at least 4 clinical trials, and a subject stays in one of the at least 4 clinical trials for as long as the medicament in that clinical trial shows efficacy in the subject, or the subject can be placed into a different one of the at least 4 clinical trials when the medicament shows decreased or no efficacy. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, an in vitro diagnostic test may not be approved or cleared by a regulatory agency. In some embodiments, from about 1 to about 8,000 subjects may not show efficacy when a medicament is administered. In some embodiments, from about 1% to about 50% or subjects may not show efficacy when a medicament is administered.

Also disclosed herein are methods that can comprise administering a medicament to a subject enrolled in a clinical trial, wherein at least 4 clinical trials are conducted simultaneously, and wherein: each of the at least 4 clinical trials are directed towards the same disease or condition, the medicament in each of the at least 4 clinical trials can be different, the at least 4 clinical trials run for at least about 10 years, and the at least 4 clinical trials have the same sponsor. In some embodiments, each of the least 4 clinical trials can be a Phase IV or a Phase III clinical trial. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, an in vitro diagnostic test may not be approved or cleared by a regulatory agency. In some embodiments, from about 1 to about 8,000 subjects may not show efficacy when a medicament is administered. In some embodiments, from about 1% to about 50% or subjects may not show efficacy when a medicament is administered.

Also disclosed herein are methods that can comprise administering a medicament to a subject enrolled in a clinical trial, wherein at least 3 clinical trial arms are run in parallel, and wherein: each of the at least 3 clinical trial arms employs a medicament, the medicament in each of the at least 3 clinical trial arms are the same, the medicament can be a non-licensed and non-approved medicament, the medicament in each of the at least 3 clinical trial arms are administered via a different administration paradigm; and a subject stays in one of the at least 3 clinical trial arms when the medicament shows efficacy in the subject, or the subject can be placed into a different one of the at least 3 clinical trial arms when the medicament shows decreased or no efficacy. In some embodiments, at least one clinical trial arm employs at least one additional medicament, wherein the at least one additional medicament can be different than the medicament. In some embodiments, the subject can be administered the medicament by a different route of administration or a different dosing schedule. In some embodiments, each of the least 3 clinical trial arms can be a Phase IV or a Phase III clinical trial. In some embodiments, the method further comprises joining the at least 3 clinical trial arms into a single clinical trial. In some embodiments, a method can further comprise conducting a diagnostic test. In some embodiments, a diagnostic test can be an in vitro diagnostic test. In some embodiments, a diagnostic test can be a theragnostic test. In some embodiments, a diagnostic test can be for efficacy. In some embodiments, an in vitro diagnostic test may not be approved or cleared by a regulatory agency. In some embodiments, from about 1 to about 8,000 subjects may not show efficacy when a medicament is administered. In some embodiments, from about 1% to about 50% or subjects may not show efficacy when a medicament is administered.

Also disclosed herein are methods of slowing a progression of rheumatoid arthritis in a subject enrolled in a clinical trial that can comprise: separating the subject into a subject pool subset based on a selection from the group consisting of: Immunoglobulin G (IgG) rheumatoid factor (RF)+/−, IgG Anti Citrullinated Protein Antibodies (ACPA)+/−, Immunoglobulin A (IgA) RF+/−, IgA ACPA+/−, C-Reactive Protein (CRP)Hi/Lo, fibrinogen (cFib)+/−; and Fc gamma receptor (FcGR)-3A, FcGR-2A, FcGR-3B, and c fragment of IgA receptor (FcAR) polymorphisms; administering a first medicament to the subject, wherein the first medicament can be a non-licensed and non-approved drug that can be biosimilar to a licensed and approved drug; administering a second medicament to the subject in the same clinical trial if the subject does not achieve remission or the first medicament does not slow the progression of rheumatoid arthritis after a time period of about 1 year; wherein the second medicament can be a non-licensed and non-approved drug that can be biosimilar to a licensed and approved drug; wherein the second medicament may not be the same as the medicament; and wherein the subject does not achieve remission or the first medicament does not slow the progression of rheumatoid arthritis in the subject after a time period of about 1 year.

Also disclosed herein are methods that can comprise: obtaining a subject pool that can comprise subjects having or suspected of having a disease or condition, wherein the subject pool can be referred by a payer; compiling a formulary, wherein the formulary comprises: approved or licensed medicaments; and non-approved or non-licensed medicaments; storing the formulary in electronic format into a formulary system that comprises a computer readable memory configured to store the formulary on an electronic storage device; stratifying the subject pool into subjects who will enroll in a clinical trial or subject who will receive a conventional treatment paradigm, wherein the stratifying comprises: consulting the formulary system to access the formulary; performing an assay on samples from subjects, or obtaining data derived therefrom, to determine a disease state genotype or immunological feature; and providing a prior authorization, wherein the prior authorization can be provided based at least in part on the assay of data derived therefrom and comprises: enrolling subjects in the clinical trial to receive a non-licensed and non-approved medicament from the formulary; or providing a conventional treatment from the formulary, wherein the subjects do not participate in the clinical trial; administering the non-licensed and non-approved medicament to subjects enrolled in the clinical trial; administering an additional medicament to the subject if the subjects that do not achieve remission after a time period of about 1 year; wherein the additional medicament can be a non-licensed and non-approved drug that may not be the same as the medicament; and entering data obtained from the clinical trial into a clinical trial system that comprises: a computer readable memory storing on an electronic storage device a database that can comprise the clinical trial data, or a summary thereof, in computer readable format; and a computer processor, wherein the computer processor can be configured to access the clinical trial data from the computer readable memory.

Also disclosed herein are methods of increasing safety, efficacy, or safety and efficacy of a medicament that can comprise employing a method of administering as described herein.

Also disclosed herein are databases that can comprise data, where the data can comprise payer data and efficacy data for a medicament, where the efficacy data can indicate that the medicament did not show efficacy in at least one subject, and where the payer data can indicate that a provider may not be reimbursed for the medicament for the at least one subject. In some embodiments, the payer data can comprise a payment received by the provider. In some embodiments, a payer data can comprise an identification of a payer. In some embodiments, a payer can be a party who does not at least partially own or have licensed intellectual property to the medicament, its formulation, or its method of use. In some embodiments, intellectual property can comprise rights in a patent or a patent application. In some embodiments, the database may not be subject to regulatory clearance, licensing, or approval by a regulatory agency. In some embodiments, the regulatory agency can be the US Federal Drug Administration (FDA), the European Medicines Agency (EMA), the Chinese FDA, or the Pharmaceuticals and Medical Devices Agency (PMDA). In some embodiments, the database can be a research tool. In some embodiments, a user of the database is not a sponsor or owner, before a regulatory agency, of the database or its architecture. In some embodiments, a computer network can be interfaced with the database. In some embodiments, the database can be attached to a specialty pharmacy.

Also disclosed herein are databases that can comprise data, where the data can comprise payer data and efficacy data for a medicament, where the medicament can be approved or licensed for a first condition, where the efficacy data can indicate that the medicament shows efficacy for a second condition that may not be the first condition in at least one subject, and where the payer data may indicate that a provider is reimbursed for the medicament for the at least one subject. In some embodiments, the payer data can comprise a payment received by the provider. In some embodiments, a payer data can comprise an identification of a payer. In some embodiments, a payer can be a party who does not at least partially own or have licensed intellectual property to the medicament, its formulation, or its method of use. In some embodiments, intellectual property can comprise rights in a patent or a patent application. In some embodiments, the database may not be subject to regulatory clearance, licensing, or approval by a regulatory agency. In some embodiments, the regulatory agency can be the US Federal Drug Administration (FDA), the European Medicines Agency (EMA), the Chinese FDA, or the Pharmaceuticals and Medical Devices Agency (PMDA). In some embodiments, the database can be a research tool. In some embodiments, a user of the database is not a sponsor or owner, before a regulatory agency, of the database or its architecture. In some embodiments, a computer network can be interfaced with the database. In some embodiments, the database can be attached to a specialty pharmacy.

Also disclosed herein are databases that can comprise data, where the data can comprise: i) subject identification data (which may be encrypted or coded) for subjects in a subject pool having or suspected of having a disease or condition, and ii) assay data on samples from subjects in the subject pool; where the database can comprise a stratifying algorithm that is performed based on the assay and can comprise enrolling subjects to receive a medicament, where the enrolling can comprise admission to a series of clinical trials conducted simultaneously, or admission to a multi-arm clinical trial conducted simultaneously, where a third-party sponsor of the series of clinical trials and the multi-arm clinical trial may be the same, and where the medicament can be licensed and approved but may not be marketed in the territory that the medicament is licensed and approved in. In some embodiments, the database may not be subject to regulatory clearance, licensing, or approval by a regulatory agency. In some embodiments, the regulatory agency can be the US Federal Drug Administration (FDA), the European Medicines Agency (EMA), the Chinese FDA, or the Pharmaceuticals and Medical Devices Agency (PMDA). In some embodiments, the database can be a research tool. In some embodiments, a user of the database is not a sponsor or owner, before a regulatory agency, of the database or its architecture. In some embodiments, a computer network can be interfaced with the database. In some embodiments, the database can be attached to a specialty pharmacy.

Also provided are methods of using data collected in the course of, or pursuant to, a study described herein. Such data can encompass clinical data, treatment data, critical clinical parameters data including staging and grading of a disease, biomarkers, progression-free survival, and the like. This may be related to treatment of an individual subject, e.g., where data are used to make treatment decisions of that subject (or other like subjects), concurrently or in the future. Thus, e.g., diagnostic data collected on a subject lead to treatment decisions for that patient or such similar patients collectively referred to as a stratified subset. This may result from diagnosis (continuing or newly determined) of the subject, or recognition that a subject is in a differentiated set of patients, e.g., patient subsets or disease subtypes. Alternatively, the data collected may be useful for the insurer, which may be assignment to insurance subsets, e.g., whether or to what an extent insurance coverage or prior authorization may be approved. Or the statistical data may be used to determine financial risk factors for various subsets of patients. All these might be applicable to the drug before product approval for commercialization, or after approval.

Also disclosed herein are databases comprising data, where the data can comprise clinical trial data for at least 3 clinical trial arms run in parallel, where each of the at least 3 clinical trial arms can employ a medicament, the medicament in each of the at least 3 clinical trial arms can be the same, the medicament can be a non-licensed and non-approved medicament, the medicament in each of the at least 3 clinical trial arms can be administered via a different administration paradigm; and i) a subject can stay in one of the at least 3 clinical trial arms when the medicament shows efficacy in the subject, or ii) the subject can be placed into a different one of the at least 3 clinical trial arms when the medicament shows decreased or no efficacy. In some embodiments, the database may not be subject to regulatory clearance by a regulatory agency. In some embodiments, the regulatory agency can be the US Federal Drug Administration (FDA), the European Medicines Agency (EMA), the Chinese FDA, or the Pharmaceuticals and Medical Devices Agency (PMDA). In some embodiments, the database can be a research tool. In some embodiments, a user of the database is not a sponsor or owner, before a regulatory agency, of the database or its architecture. In some embodiments, a computer network can be interfaced with the database. In some embodiments, the database can be attached to a specialty pharmacy.

Also disclosed herein are databases that can comprise data, wherein the data can comprise clinical trial data for at least 4 clinical trials conducted simultaneously, where: each of the at least 4 clinical trials can be directed towards the same disease or condition, the medicament in each of the at least 4 clinical trials can be different, the at least 4 clinical trials can run for at least about 10 years, and the at least 4 clinical trials can have the same sponsor. In some embodiments, the database may not be subject to regulatory clearance by a regulatory agency. In some embodiments, the regulatory agency can be the US Federal Drug Administration (FDA), the European Medicines Agency (EMA), the Chinese FDA, or the Pharmaceuticals and Medical Devices Agency (PMDA). In some embodiments, the database can be a research tool. In some embodiments, a user of the database is not a sponsor or owner, before a regulatory agency, of the database or its architecture. In some embodiments, a computer network can be interfaced with the database. In some embodiments, the database can be attached to a specialty pharmacy.

Also disclosed herein are systems that can comprise a pharmacy and an formulary, wherein the system can be employed to treat a population of subjects that can comprise a first and a second plurality of subjects by at least two different treatment paradigms, wherein the first plurality of subjects are enrolled in a first treatment paradigm and the second plurality of subjects are enrolled in a second treatment paradigm, wherein the first treatment paradigm comprises administering a licensed, approved, or licensed and approved medicament to treat a disease or condition indicated for the licensed, the approved, or the licensed and approved medicament; and the second treatment paradigm comprises administering an unlicensed and unapproved medicament to treat the same disease or condition, wherein the unlicensed and unapproved medicament can be administered with an assurance to a payer based on efficacy on a subject-by-subject basis; wherein the first and second plurality of subjects are different. In some embodiments, a pharmacy comprises unlicensed; unapproved; approved; licensed; and licensed and approved medicaments. In some embodiments, a payer can be a party who does not at least partially own or have licensed intellectual property to the medicament, its formulation, or its method of use. In some embodiments, intellectual property comprises rights in a patent or a patent application. In some embodiments, a system can further comprise a communication device operatively coupled to the system. In some embodiments, a system can further comprise a peripheral component operatively coupled to a power supply, wherein the peripheral component is selected from the group consisting of a keyboard, a computer screen, a router, a USB cable, a computer terminal, a computer terminal screen, and any combination thereof. In some embodiments, a system can be configured to transmit or receive data. In some embodiments, a system can be in communication with an external device. In some embodiments, an external device can be a smart phone, or similar device. In some embodiments, a system may not be subject to regulatory clearance, licensing, or approval by a regulatory agency. In some embodiments, a regulatory agency can be the US Federal Drug Administration (FDA), the European Medicines Agency (EMA), the Chinese FDA, or the Pharmaceuticals and Medical Devices Agency (PMDA). In some embodiments, a system may be previously been exempted, cleared, licensed, or approved by a regulatory agency. In some embodiments, a regulatory agency can be the US Federal Drug Administration (FDA), the European Medicines Agency (EMA), the Chinese FDA, or the Pharmaceuticals and Medical Devices Agency (PMDA). In some embodiments, a system can be a research tool. In some embodiments, a user of the system may not be a sponsor or owner, before a regulatory agency, of the system, its architecture, or its hardware. In some embodiments, a system can be configured to transmit or receive data. In some embodiments, a system can be connected to a computer network. In some embodiments, a system can be configured to display data on a computer screen operatively coupled to the system. In some embodiments, data can comprise clinical trial data. In some embodiments, data can comprise payer data. In some embodiments, data can comprise efficacy data.

Also disclosed herein is computer readable memory storing at least transiently on an electronic storage device a database that can comprise clinical trial data, or a summary thereof, in computer readable format obtained from a clinical trial employing a method of administering as described herein. In some embodiments, a database further comprises diagnostic clinical trial data. In some embodiments, a diagnostic clinical trial data can be theragnostic data.

Also disclosed herein is computer readable (or otherwise accessible) memory storing at least transiently on, e.g., an electronic (including an electrooptical) storage device a database that contains observations or data or information collected, e.g., in the steps of a method described herein.

Also disclosed herein are systems that can comprise a computer readable memory as described herein and a computer processor, wherein the computer processor can be configured to access the clinical trial data from the computer readable memory. In some embodiments, a system can further comprise a communication device operatively coupled to the system. In some embodiments, a system can further comprise a peripheral component operatively coupled to a power supply, wherein the peripheral component is selected from the group consisting of a keyboard, a computer screen, a router, a USB cable, a computer terminal, a computer terminal screen, and any combination thereof. In some embodiments, a system can be configured to transmit or receive data. In some embodiments, a system can be in communication with an external device. In some embodiments, an external device can be a smart phone, or similar device. In some embodiments, a system may not be subject to regulatory clearance, licensing, or approval by a regulatory agency. In some embodiments, a regulatory agency can be the US Federal Drug Administration (FDA), the European Medicines Agency (EMA), the Chinese FDA, or the Pharmaceuticals and Medical Devices Agency (PMDA). In some embodiments, a system may be previously been exempted, cleared, licensed, or approved by a regulatory agency. In some embodiments, a regulatory agency can be the US Federal Drug Administration (FDA), the European Medicines Agency (EMA), the Chinese FDA, or the Pharmaceuticals and Medical Devices Agency (PMDA). In some embodiments, a system can be a research tool. In some embodiments, a user of the system may not be a sponsor or owner, before a regulatory agency, of the system, its architecture, or its hardware. In some embodiments, a system can be configured to transmit or receive data. In some embodiments, a system can be connected to a computer network. In some embodiments, a system can be configured to display data on a computer screen operatively coupled to the system. In some embodiments, data can comprise clinical trial data. In some embodiments, data can comprise payer data. In some embodiments, data can comprise efficacy data. In some cases, data can be transmitted by an email.

Also disclosed herein are smartphones, or similar devices, that can comprise data from a database described herein. Also disclosed herein are personal digital assistants that can comprise data from a database as described herein.

Also disclosed herein is computer readable memory storing at least transiently on, e.g., an electronic storage device an electronic regulatory submission (ERS), or a section thereof, in computer readable format, the electronic regulatory submission containing clinical trial data or a summary thereof, e.g., obtained from a clinical study or trial employing a method of administering as described herein.

Also disclosed herein is computer readable memory storing at least transiently on an electronic storage device an electronic regulatory submission, or a section thereof, in computer readable format, the electronic regulatory submission containing the steps of a method of administering as described herein.

Also disclosed herein is computer readable memory storing at least transiently on an electronic storage device a regulatory submission application in computer readable format seeking approval for label revision of a drug, the regulatory submission application containing clinical trial data or a summary thereof obtained from a clinical trial employing a method of administering as described herein. In some embodiments, clinical trial data comprises data from a diagnostic, and wherein the label revision requires administration of the diagnostic and evaluation before the medicament can be properly or optimally administered.

Also disclosed herein is computer readable memory storing on an electronic storage device a regulatory submission application in computer readable format seeking approval for label revision of a drug, the regulatory submission application contains the steps of a method of administering as described herein.

Also disclosed herein are kits that can comprise a drug and an approved label revision approved based in part on a regulatory agency utilizing computer readable memory as described herein. In some embodiments, a drug can be comprised in a container.

Also disclosed herein are kits that can comprise a drug in a container, and a package insert, wherein the package insert contains the data, a summary thereof, or the steps thereof, of a method of administering as described herein, or any combination thereof.

Also disclosed herein are label revised drugs that was developed under an exemption to patent protection by conducting a method of administering as described herein. In some embodiments, a label revised drug can be approved by a regulatory agency. In some embodiments, a label revised drug can be approved for administration through prior authorization.

Also disclosed herein is computer readable memory storing on an electronic storage device a drug formulary, in computer readable format that can comprise a label revised drug as described herein. In some embodiments, a label revised drug can be approved for administration through prior authorization.

Also disclosed herein is computer readable memory storing on an electronic storage device a drug formulary, in computer readable format that can comprise a medicament that may not be licensed or approved by a regulatory agency. In some embodiments, a medicament may only be authorized in a clinical trial.

Also disclosed herein are systems that can comprise a computer readable memory as described herein and a computer processor, wherein the computer processor can be configured to access the drug formulary from the computer readable memory. In some embodiments, a system can further comprise a communication device operatively coupled to the system. In some embodiments, a system can further comprise a communication device operatively coupled to the system. In some embodiments, a system can further comprise a peripheral component operatively coupled to a power supply, wherein the peripheral component is selected from the group consisting of a keyboard, a computer screen, a router, a USB cable, a computer terminal, a computer terminal screen, and any combination thereof. In some embodiments, a system can be configured to transmit or receive data. In some embodiments, a system can be in communication with an external device. In some embodiments, an external device can be a smart phone, or similar device. In some embodiments, a system may not be subject to regulatory clearance, licensing, or approval by a regulatory agency. In some embodiments, a regulatory agency can be the US Federal Drug Administration (FDA), the European Medicines Agency (EMA), the Chinese FDA, or the Pharmaceuticals and Medical Devices Agency (PMDA). In some embodiments, a system may be previously been exempted, cleared, licensed, or approved by a regulatory agency. In some embodiments, a regulatory agency can be the US Federal Drug Administration (FDA), the European Medicines Agency (EMA), the Chinese FDA, or the Pharmaceuticals and Medical Devices Agency (PMDA). In some embodiments, a system can be a research tool. In some embodiments, a user of the system may not be a sponsor or owner, before a regulatory agency, of the system, its architecture, or its hardware. In some embodiments, a system can be configured to transmit or receive data. In some embodiments, a system can be connected to a computer network. In some embodiments, a system can be configured to display data on a computer screen operatively coupled to the system. In some embodiments, data can comprise clinical trial data. In some embodiments, data can comprise payer data. In some embodiments, data can comprise efficacy data.

Also disclosed herein are pharmacies that can comprise a system as described herein and a physical storage of medicaments, wherein at least some of the medicaments in the physical storage are recited in the system. In some embodiments, a physical storage of medicaments and the system are both present in a same building. In some embodiments, a system can be configured to be accessed in the same building as the physical storage.

Also disclosed herein are pharmacies that can comprise a label revised drug as described herein in a container. In some embodiments, a pharmacy can further comprise a medicament that may not be licensed and not approved by a regulatory agency. In some embodiments, a medicament can be biosimilar to a medicament that is approved and licensed. In some embodiments, a medicament has a commercial cost that can be from about 50% to about 100% of a commercial cost of the medicament that is approved and licensed. In some embodiments, a medicament can be a protein. In some embodiments, a protein has a sequence that can be at least about 95% homologous to a corresponding licensed and approved medicament. In some embodiments, a pharmacy can further comprise a medicament that can be a specialty drug; wherein the specialty drug treats a complex, chronic, rare, or difficult to manage disease or disorder. In some embodiments, a pharmacy can further comprise a medicament that can be interchangeable with a medicament that is approved and licensed.

Also disclosed herein are methods that can comprise: consulting a system as described herein to access the formulary; performing an assay on a sample from a subject, or obtaining data derived therefrom, to determine a disease state; and providing a prior authorization, wherein the prior authorization comprises: enrolling the subject in a clinical trial to receive a non-licensed and non-approved medicament recited in the formulary from a pharmacy; or providing a conventional treatment, wherein the subject does not participate in the clinical trial; wherein the prior authorization can be provided based at least in part on the assay or data derived therefrom.

Also disclosed herein are methods that can comprise consulting a system as described herein and selecting a medicament to be administered based on a progression of treatment. In some embodiments, a clinical trial provider who can be at a different location than the subject consults the system. In some embodiments, a method can further comprise a process for administering the medicament to the subject. In some embodiments, a specialty distributor may not be involved in the process for administering to the subject. In some embodiments, a pharmacy benefit manager (PBM) may not be involved in the process for administering to the subject.

Also disclosed herein are methods of treating, slowing, or preventing the progression of a disease or condition that can comprise employing a method of administering as described herein.

Also disclosed herein are methods of treating, slowing, or preventing the progression of a disease or condition that can comprise employing a system as described herein.

Also disclosed herein are methods of treating, slowing, or preventing the progression of a disease or condition that can comprise employing a computer readable memory as described herein.

Also disclosed herein are methods that can comprise administering a non-approved and non-licensed medicament by employing a method of administering as described herein, wherein the non-licensed and non-approved medicament was taken to trial but discontinued; and generating data or licensing for the non-licensed and non-approved medicament based at least in part from a clinical trial.

Also disclosed herein are methods that can comprise administering a medicament to a subject in a clinical trial, wherein at least about 16,000 subjects are enrolled in the clinical trial, wherein the clinical trial can be in the US, and wherein the clinical trial can be conducted for at least 7 years. In some embodiments, a medicament may not be a prophylactic vaccine.

Also disclosed herein are methods that can comprise administering a medicament that can be a specialty drug to a subject in a clinical trial, wherein at least about 15,000 subjects are enrolled in the clinical trial, wherein the clinical trial can be in the US, wherein the clinical trial can be conducted for at least 7 years; and wherein the specialty drug treats a complex, chronic, rare, or difficult to manage disease or disorder. In some embodiments, a provider of the clinical trial can be paid at least about $2,000 in medicament cost for a month of treatment on a subject-by-subject basis only if the medicament shows efficacy. In some embodiments, a clinical trial can be a prospective clinical trial.

Also disclosed herein are methods of gaining exemption from infringement of a US patent with claims covering a patented technology that can comprise using the patented technology solely for uses reasonably related to the development and/or submission of information, e.g., non-routine submission of data or results, under a Federal law which regulates the manufacture, use, offer to sell, or sale of drugs or veterinary biological products. In some embodiments, a use reasonably related to the development or submission of information under a Federal law which regulates the manufacture, use, offer to sell, or sale of drugs or veterinary biological products can be a clinical trial. In some embodiments, a method can further comprise administering a medicament to a subject in a clinical trial within the US, wherein a clinical trial participation cost can be paid by a third-party payer on a subject-by-subject basis based on efficacy, wherein the payer pays on a pay-as-you-go basis or in multiple installments, wherein at least one subject does not show efficacy, wherein the medicament is recited in Table 1, 2, or 3; or listed in the FDA Orange, Purple Book, or a foreign counterpart thereof; or wherein the payer can be a party who does not at least partially own or have licensed intellectual property to the medicament, its formulation, or its method of use. In some embodiments, the financial and/or legal liabilities related to the clinical trial may rest with the third-party payer.

Also disclosed herein are methods of gaining exemption from infringement of a European or other patent with claims covering a patented technology that can comprise using the patented technology solely for uses reasonably related to the development or submission of information under a Bolar Exception which regulates the manufacture, use, offer to sell, or sale of drugs or veterinary biological products.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 depicts treatment of a cohort of patients by employing diagnostics.

FIG. 2 depicts stratification of a cohort of patients into either conventional treatment or treatment in a clinical trial.

FIGS. 3A and 3B depict platforms that can be used to stratify a patient population into either receiving a conventional treatment or entering into a clinical trial.

FIG. 4 depicts an extended clinical trial for treatment of a rheumatoid arthritis (RA) condition after being selected to enter into the clinical trial.

FIG. 5 depicts an exemplary system workflow for use in conducting a clinical trial as described herein.

FIG. 6A-6E illustrate various features of a clinical trial (or clinical study). FIG. 6A illustrates key features, which may be individually or combined in a trial or study, which may have the exemption (immunity, or safe harbor) provision (from patent infringement, codified in relevant part at 35 U.S.C. § 271(e)), or where the exemption (safe harbor) provision is not required (non-271(e) setting). Notable features can include: (i) the pay-to-participate trial is funded, at least in part, by a third-party payer, or by patients; (ii) the trial is sponsored by a third-party (e.g., a managed care company); (iii) the trial is significantly large (e.g., N=20,000) and/or significantly long (e.g., 7-10 years); (iv) the trial is performed either from a single clinical site (e.g., virtual trial guided by digital health, with or without augmented reality technologies); (v) where the trial is a phase-4 clinical trial; and (vi) with an objective of providing N=1 patient-specific efficacy and/or N=1 patient-specific financial assurances, e.g., reinsurance. Combinations of these features, in all assortments, can be employed to conduct such a clinical trial, as exemplified in FIG. 6B-E.

FIG. 7 shows the entities and/or stakeholders involved in data development and usage.

### DETAILED DESCRIPTION

Overview

Disclosed herein are methods of conducting clinical trials. For instance, a method can comprise enrolling a large number of subjects in a clinical trial. In some cases, a clinical trial can be conducted for at least about 7 years. In some cases, a clinical trial can be held in the United States. In some cases, a medicament can be administered in a clinical trial. In some cases, the medicament may not be a prophylactic vaccine. In some cases, a medicament can be a specialty drug.

Also disclosed herein in some embodiments are methods of conducting clinical trials that can include a payer, e.g., a third-party payer, who pays for the participation of a subject in a clinical trial. In some cases, a payer can pay for participation on a subject-by-subject basis, or alternatively on a collective group basis. In some cases, a clinical trial provider may only be paid for a clinical trial when the medicament shows efficacy in a subject, or may pay less when efficacy is not achieved. In some embodiments, the financial and/or legal liabilities related to the clinical trial may rest with the third-party payer.

Also disclosed herein in some embodiments are methods of performing multiple clinical trials, or methods of performing clinical trials with multiple arms. In some cases, a method can comprise administering a non-licensed and non-approved medicament to a subject in a clinical trial. In some cases, the clinical trials can last at least about 10 years.

Also disclosed herein in some embodiments are methods of generating clinical trial data and entry of such data in a database by performing methods as described herein, as well as computer readable memory capable of storing the data, and systems comprising computer readable memory.

Also disclosed herein in some embodiments are regulatory submission applications in electronic or other format containing data from a clinical trial as described herein. In some instances, a regulatory application can be used to seek approval for a label revision of a drug.

Also disclosed herein in some embodiments are kits that can comprise a drug and an approved label revision resulting from a regulatory submission application as described herein.

Also disclosed herein in some embodiments are electronic formularies that can comprise a label revised drug as described herein.

Also disclosed herein in some embodiments are pharmacies that can comprise a physical storage of a label revised drug as described herein.

Also disclosed herein in some embodiments are methods of selecting a subject for a clinical trial or conventional treatment that can comprise consulting a formulary as described herein, performing an assay on a subject, and administering a drug from a pharmacy.

Also disclosed herein in some embodiments are methods of selecting a medicament to be administered to a subject by consulting a clinical trial system as described herein.

Also disclosed herein in some embodiments are methods of slowing the progression of a disease or condition by performing methods as described herein.

Also disclosed herein in some embodiments are methods of administering non-approved and non-licensed medicaments to subject in a clinical trial.

Also disclosed herein in some embodiments are businesses utilizing any combination of these features.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “subject”, “patient” or “individual” as used herein can encompass a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. In some cases, a mammal can be a display animal, a breeding animal, a companion animal, an endangered species, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old, with some subsets being above or below ages 30, 40, 50, 60, 70, 80, 90, 100 in various combinations. In some instances, a subject can be a male. In some instances, a subject can be a female. In some instances, a female may be pregnant. In some cases, a subject may be a chimera or hybrid. In some instances, a subject can be diagnosed with, or can be suspected of having, a condition or disease. In some cases, a subject may be without a condition or disease. Various stratification criteria may exist, e.g., height, weight, sex, age, other differentiating or relevant features, health statuses, medical histories, genetic features, immunological conditions, and the like. In some instances, a disease or condition can be cancer. A subject can be a patient. A subject can be an individual. In some instances, a subject, patient or individual can be used interchangeably. In some cases, a subject can be a member of a subject pool. In some cases, a “subject” and a “subject pool” can be used interchangeably.

The term “third-party payer” or “payer” herein can refer to an entity that funds a significantly larger-sized clinical trial, e.g., a sizeable clinical trial of N=30000, and bears a substantial component of the clinical trial costs including medicament and/or treatment costs. A patient may pay a component of the drug cost, e.g., coinsurance amount if it is a specialty drug; copay amount if it is a non-specialty drug. In some instance, a patient may pay a component of the clinical trial costs, e.g., about 3% of the per-patient clinical trial cost, about 7% of the per-patient clinical trial cost, or in excess of about 20%, e.g., about 30%, 40%, 50%, 60%, 70%, 80%, 90%, etc., can be paid. Such trials are herein referred to as ‘pay-to-participate’ trials. In some embodiments, a pharmaceutical company that developed, manufactured, or commercialized the drug is not a third-party payer. A third-party payer can be a private insurance company, a government entity (e.g., a government healthcare entity such as U.S. Medicare system, UK PHS), a private payer, an employer or a consortium of employers, a pension fund, and the like. A payer also may encompass a managed care company. In one embodiment, a fully-integrated payer may provide additional functions that can be offered by a managed care company, e.g., pharmacy benefit management (PBM), specialty pharmacy, disease and therapy management, theragnostics, and the like. In some embodiments, a fully-integrated payer can be a non-profit managed care consortium, e.g., Kaiser Permanente. In some embodiments, such a managed care company may also provide fully-integrated clinical trial capabilities and or healthcare provider capabilities. In another embodiment, such a managed care company, through vertical or lateral integration with a large pharmaceutical company, may provide fully-integrated clinical trial capabilities and or healthcare provider capabilities. Traditionally, clinical trials are funded by either pharmaceutical companies (who is also the clinical trial sponsor) or successors of interest. Disease-specific foundations, e.g., Michael J. Fox Foundation for Parkinson's Research; government agencies, e.g., National Institute of Health, also fund clinical trials albeit of much smaller sizes e.g., N=10-200. Historically, traditional sponsors have not conducted such sizeable trials.

In some embodiments, trials of significantly larger size than the typical, limited, patients-funded, pay-to-participate trials, are envisioned. In particular, trials of larger size, distinguishable as “sizeable” from earlier “limited” trials of about 70 subjects (N=70), e.g., about 80, 90, 100, 110, 120, 130, 140, 150, 170, 190, 210, etc., can be included. In one embodiment, a sizeable trial may involve 30,000 subjects. In some embodiments, this can include trials comparing, e.g., biosimilar or small molecule generics as alternative to a reference biologic or reference small molecule, including a reference drug from Table 1, 2, or 3. In various such sizeable trials, larger percentages of participants have clinical trial costs paid by such third-party payers, e.g., in excess of about 70%, e.g., about 75, 80, 85, 90, 95, 97, 99, etc. In some cases, patients enrolled in the trial pay for drug costs and/or trial costs, e.g., 3% of the per-patient trial cost, or in excess of about 30%, e.g., about 40%, 50%, 60%, 70%, 80%, 90%, etc.

The terms “third-party clinical trial sponsor” or “sponsor” or “clinical trial provider” herein can refer to an entity that conducts a clinical trial. In one embodiment, a managed care company sponsors a sizeable clinical trial and may employ telehealth architecture to conduct such sizeable clinical trials, e.g., single-site, virtual or site-less clinical trials. In embodiments, a pharmaceutical company that developed, manufactured, or commercialized the drug is not a sponsor, nor will it have direct access to the clinical trial data. In some embodiments, a fully-integrated third-party payer, e.g., a payer with managed care capabilities, or a managed care company can be a sponsor. Such a sponsor is responsible for initiating, managing, funding, and/or otherwise ensuring that the clinical trial is properly performed, complying with safety, ethical, statistical, reproducibility, and other aspects of a clinical trial. In some embodiments, a fully-integrated provider can be a non-profit managed care consortium, e.g., Kaiser Permanente. Traditionally, a sponsor may include a pharmaceutical company, a patient advocacy foundation, or an academic clinical investigator involved in investigator-initiated trials, e.g., limited trials. In some embodiments, a sponsor can be an entity which tests a biosimilar or generic counterpart or equivalent to a reference biologic or small molecule drug, e.g., including a reference drug from Table 1, 2, or 3; or listed in the FDA Orange, Purple Book, or a foreign counterpart thereof.

The term “trial site” can refer to an investigational site. Traditionally, large trials can occur from multi-site trials but implement the same clinical protocol. These investigational sites can be administratively distinct from one another. Smaller trials, e.g., N=100, may occur from a single-site trial, which utilizes one investigational site to conduct and coordinate the protocol. In one embodiment, a managed care company is the third-party sponsor of a sizeable clinical trial, e.g., single-site, virtual or site-less clinical trials. In some embodiments, a managed care company with offices at multiple locations but has a single clinical trial protocol can be considered a single site. Participants may receive intervention and/or outcome assessments under the direction of one investigational site. In some embodiments, a virtual, or site-less, trial can conduct such clinical trials. In some embodiments, a mobile or teledevice allows trial participation and might minimize or dispense with actual travel to a clinical research facility. Wearable or available sensors may measure and record diagnostic data on a participant, which serves as one means to assist in the intervention and/or outcomes assessment. Devices may be connected to monitor parameters such as body temperature, blood glucose, immunological measures and status, joint flexibility, and other evaluation criteria. In some embodiments, the device may include wearable devices, watch-like devices, implants, spectacle-like devices, and the like, and the collected data may be automatically relayed to the care team, e.g., recorded in electronic data capture (EDC) systems. In some embodiments, study personnel may visit participants, e.g., at home, to assist or verify drug administration compliance and follow-up. Communication and feedback may be provided at all stages from recruitment, informed consent, patient counseling, through to answering clinically and therapeutically relevant questions and measuring clinical endpoints and adverse reactions and explaining participant outcomes. A uniform diagnostic or theragnostic laboratory for patient evaluations will provide uniformity in therapeutic evaluation of patients dispersed geographically.

The term “N=1 efficacy” herein can refer to achieving therapeutic efficacy for a given patient in a given disease indication. Such efficacy can include different cut off scores, e.g., remission, cure, excellent response for a given disease, e.g., multiple sclerosis; progression-free survival in cancers, e.g., B-NHL. In one embodiment, sizeable trials may provide N=1 efficacy. Irrespective of the size of a sizeable clinical trial, in N=1 efficacy, efficacy outcome is specific to that subject in the trial.

The term “N=1 assurance” herein can refer to providing patient-specific efficacy assurance and or financial assurance for a given patient participating in a trial for a specified disease indication (e.g., rheumatoid arthritis). In one embodiment, such a sizeable trial generates N=1 assurance.

The term “assurance” herein can refer to a form of guarantee, e.g., therapeutic guarantee, product guarantee, service guarantee, financial guarantee, and the like. Assurance can also be referred to herein as reinsurance. Assurance can limit the amount of loss a third-party payer, e.g., Medicare or an employer, who can potentially suffer from uncovered losses.

The term “clinical trial merger” herein can refer to a context wherein a clinical trial and treatment paradigms are seamlessly overlapping. In some embodiments, said clinical trial is a phase-3 clinical trial, or a phase-4 clinical trial. In some embodiments, said phase-3 clinical trial is wholly funded by a third-party payer. In some embodiments, said phase-4 clinical trial is wholly funded by a third-party payer. In some embodiments, said phase-3 or -4 clinical trials are conducted by a third-party sponsor, e.g., a managed care company. In some embodiments, said clinical trials are sizeable trials, larger than limited trials. In some embodiments, said phase-4 clinical trial tests an FDA-approved drug in an approved disease indication, and that trial employs 40000 subjects insured by a third-party payer. Such a clinical trial merger meets two objectives: a sizeable clinical trial is conducted to generate necessary clinical data, e.g., long-term disease remission and disease relapse patterns, excellent versus worst responder subsets, N=1 efficacy; and (b) third-party payer can simultaneously administer drugs to its patients. Such a trial merger has significant time and/or cost savings for payers and patients. In one embodiment, the drug cost for payer is cheaper by about 20%, e.g., about 30%, 40%, 50%, 60%, 70%, 80%, etc., in the clinical trial. In some embodiments, the drug is an FDA-approved biosimilar for an approved indication. In non-271(e) clinical trial settings, the innovator, e.g., a mid-sized biotechnology company, has an option to participate in the trial, along with the third-party managed care company. The innovator has an option to pay for the clinical trial costs, e.g., about 5%, 10%, 30%, 40%, etc., of the costs. Such Phase-3 or Phase-4 clinical trials may be of conventional trial sizes, e.g., N=300-1000 for a Phase-3 trial, and the trial duration may be, e.g., 2-5 years.

An “insurer” can refer to relationships where an entity has a contractual obligation to provide medical care to a patient. In contrast to disability income insurance, medical expense coverage provides benefits for various medical services, including physician services, nursing services, hospital services, supplies, equipment, and associated costs. Often, there are a number of limits to encourage careful use and contain costs. These limits typically take the form of deductibles, coinsurance provisions, and maximum caps. Insurers may offer managed care programs that utilize preferred provider organizations (PPOs) or health maintenance organizations (HMOs). Certain plans provide specific benefits, e.g., “dread disease” policies which are directed to specific illnesses, such as cancers or autoimmune conditions. The plan may limit treatment, or specifically be directed to treatment of the designated condition. Historically and traditionally, insurers are not third-party payers of clinical trials, e.g., sizeable trials.

Hospital and medical expense associations can include, e.g., Blue Cross and Blue Shield plans and health maintenance organizations (HMOs). A Blue Cross association is a health care membership group organized by hospitals in a geographic area to provide hospital expense prepayment plans. Blue Shield associations offer analogous prepayment coverage for surgical and medical services performed by physicians. HMOs attend to emphasize preventive medicine and managed care to contain costs. HMOs can be either for-profit or not-for-profit, which depends, in part, on the nature of the sponsoring group. Historically and traditionally, providers are not third-party sponsors of clinical trials, e.g., sizeable trials.

The term “medicament” can be used to refer to an approved drug or a drug candidate that is undergoing clinical trial. A “drug” can include any therapeutic that has completed a regulatory approval process (e.g., USFDA, or a foreign counterpart). A “drug candidate” can include any therapeutic that has not completed a regulatory approval process. In some cases, a drug or a drug candidate can be a therapeutic that may be undergoing, or has undergone a Phase I, Phase II, Phase III, or a Phase IV clinical trial as defined by the USFDA. In some cases, a drug candidate can be a therapeutic that has not undergone a Phase I, Phase II, Phase III, or a Phase IV clinical trial as defined by the USFDA. These terms may also encompass various diagnostic methods, theragnostic products, medical devices, cell-based therapies and therapeutics, nucleic acids-based therapies and therapeutics, and the like.

The term “label revision” can refer to a revision to an already existing drug label approved by a regulatory agency, e.g., USFDA. A revision can be a revision to an indication, a subset of patients, a route of administration, a required companion diagnostic, a dosage, and the like. In some cases, a label revision can be a first label approved for a drug by a regulatory agency.

An “exemption to patent protection” can include an exemption to infringement of a patented invention, e.g., an approved, or licensed drug, by making, using, or selling the invention (e.g., drug) in a jurisdiction for uses reasonably related to the development and/or submission of information under a law which regulates the manufacture, use, offer to sell, sale or import of drugs or veterinary biological products. In the U.S., the law can be a Federal law, and these uses can include the purpose of development and submission of information, e.g., non-routine submission of data or results, to a regulatory agency such as the US Food and Drug Administration (e.g., a clinical trial), but may include other governmental agencies, including Federal or State, including, e.g., the US Public Health Service, the Centers for Medicare and Medicaid Services (CMS), the Agency for Healthcare Research and Quality (AHRQ), or other agency which may regulate such products. See, e.g., Russo and Johnson (2015) Cold Spring Harbor Perspect. Med. 5:a020933. Examples of such agencies in Europe can include the National Institute for Health and Care Excellence (NICE) in the United Kingdom; the Federal Joint Committee (FJC) in Germany; and the like. Examples of such Federal laws can include an exemption under 35 U.S.C. § 271(e) in the United States; a Bolar exemption in Europe (see EU Directive 2004/27/EC, Article 10(6); “Comparison of Bolar exemptions across Europe”; differences in scope across jurisdictions due to different judicial interpretations); the UK (sections 60(5)(b) Patents Act 1977; 60(5)(i) and 60(6D) and (6E)); Canada (section 55.2(1) of Patent Act; not limited to pharmaceuticals or generic medicines); Mexico (Article 22.1 of Mexican Industrial Property Law, experimental use exception; and article 167bis), Law No. 9.279/96 in Brazil; Chilean Patent Law Article 49; Andean Decision 486 and Decree 0729 in Colombia; Dominican Law 20-00 Article 30; Peruvian Decree 1075 Article 39; Law No. 17.164 Section 39 in Uruguay; Section 20(5)(e) of the Pakistani Patents Ordinance 2000; Section 107(a) of the Indian Patents Act; the Universally Accessible Cheaper and Quality Medicines Act of 2008 in the Philippines; the Malaysian Patents Act 1983 Section 37(1A); Japan (Section 69(1) experimental use exception; see Johnson (2003) Pacific Rim Law and Policy Journal Assn, Chinese Patent Law Article 69(5) and third amendment to China Patent Law approved December 2008 taking effect October 2009 (see steps and features of Lu, et al. Chapter 2, table 2.4 in Lu (ed. 2015) Approaching China's Pharmaceutical Market: A Fundamental Guide to Clinical Drug Development Springer, ISBN: 978-3-319-155-75-3; South Korea (Article 96-1 experimental use exception, may cover pharmaceuticals, biologics and generics); the Australian Patents Act 1990 Section 119A; and the New Zealand Patents Act 1953 Section 119A. See also, e.g., Tridico, et al. (2014) “Facilitating generic drug manufacturing: Bolar exemptions worldwide” WIPO Magazine issue March 2014 (June).

In some embodiments, “uses reasonably related to the development and submission of information under a Federal law which regulates the manufacture, use, or sale of drugs or veterinary biological products” can be a clinical trial. Alternatively, such development of information may be a post-approval, CMS-required or -recommended activity. In some embodiments, this will be a post-approval, AHRQ-required or -recommended activity. In some embodiments, this will be a post-approval, FDA-required activity, which may be maintained for audit, if not specifically submitted. Examples of other Federal agencies which may require such an activity can include the United States Social Security Administration, the Department of Veterans Affairs, the Centers for Disease Control and Prevention, the Department of Defense, the Department of Energy, the National Institute of Health, and the like. In some embodiments, the information that can be developed may be duly submitted to a Federal agency on a timely basis. In some embodiments, such a developed information may not be submitted to a Federal agency; however, such information can be available for Federal inspection at any time, e.g., for 1-7 years.

In some embodiments the information developed can be stored in a database, e.g., an electronic database. Any method as described herein may employ a database as a research tool (e.g., for use in a clinical trial). In some cases, a database can be a relational database. In some embodiments, the information developed can be stored in a data warehouse. In some embodiments, the information can consist of details pertaining to conventional treatment, e.g., electronic health records (EHR), claims data, payer and payment details, provider details, and in addition can include critical clinical parameters: excellent responders and poor responders to a treatment, disease remission and relapse details to a therapy, theragnostic details, staging of the disease, grading of the disease, subject-specific longitudinal treatment outcomes to a therapy, subject-specific disease progression and/or disease severity patterns, stratified subset-specific longitudinal treatment outcomes, stratified subset-specific disease progression and/or disease severity patterns, mechanistically correlated biomarkers, progression-free survival (PFS), event-free survival (EFS), and the like. Collectively, all such data combined, e.g., real-world data (RWD) and critical clinical parameters obtained from conventional treatment platform can be used to generate regulatory-grade data of clinical research quality. In some embodiments, critical clinical parameters are theragnostics guided. In some embodiments, disease and therapy management protocols are deployed to develop critical clinical parameters. In some embodiments, the information developed can be duly submitted to Federal agencies, e.g., CMS, AHRQ, and FDA (or foreign counterparts) on a timely basis. In some embodiments, the information developed can be duly submitted to FDA for label revision, e.g., label restriction. In some embodiments, the information developed can be used to develop synthetic control arms. In some embodiments, the information developed can be used to develop and provide N=1 assurance, e.g., efficacy and/or financial assurances. In some embodiments, all of these data can be stored in a data warehouse or system. In some embodiments, the information can consist of details pertaining to a clinical trial, e.g., in rheumatoid arthritis.

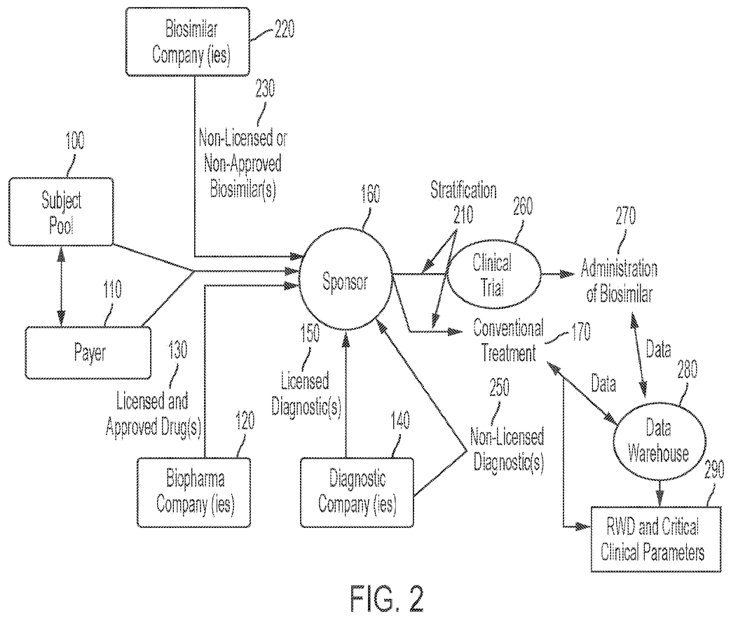

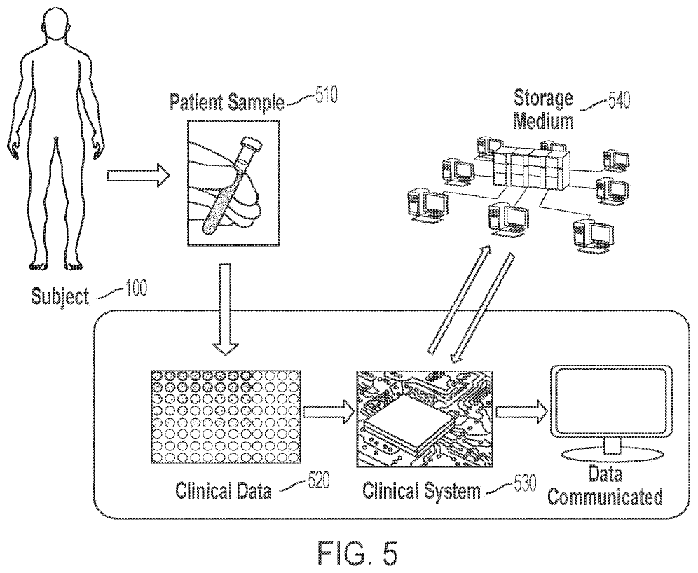

In some embodiments, developed information e.g., data in the storage medium, can be collected and archived to build a disease-specific data warehouse (FIG. 5), which can be analyzed to discover, analyze disease patterns and treatment response patterns, e.g., in subset(s) of rheumatoid arthritis, treated with drugs including, e.g., adalimumab, infliximab, etanercept, tocilizumab, and tofacitinib. For instance, in multiple sclerosis or rheumatoid arthritis, such data warehouse may contain, e.g., with 0.5 million patients, 10 million prescriptions (prior authorizations), 20 million lab results, and 10-year longitudinal treatment follow-up. In some embodiments, such a data warehouse can be integrated with both clinical trial and conventional treatment platforms (FIG. 2). A warehouse may allow recognition or identification of new subsets of conditions or disease, which may be subject to effective diagnosis and treatment. A warehouse of data may be mined using artificial intelligence tools, e.g., to develop precision medicine protocols. In some embodiments, the information that can be developed can be duly submitted to Federal agencies, e.g., CMS, AHRQ, and FDA on a timely basis. In some embodiments, such a developed information may not be submitted to Federal agencies; however, such information can be available for auditing or inspection by the Federal agencies, e.g., for 1-7 years.

The term “real-world data” (RWD) can refer to the data relating to patient health status and/or the delivery of healthcare routinely collected from a variety of sources. The term “real-world evidence” (RWE) can refer to the clinical evidence about the usage and potential benefits or risks of a medical product derived from RWD analysis (see, e.g., Framework for FDA's real-world evidence program, December 2018, www.fda.gov). Examples of RWD can include data derived from electronic health records (EHRs); medical claims and billing data; data from product and disease registries; patient-generated data, including from in-home-use settings; and data gathered from other sources that can inform on health status, such as mobile devices.

The term “synthetic control arm” can include data collected from subjects enrolled in conventional treatment platform, e.g., real-world data that has previously been collected from sources such as health data generated during routine care, including electronic health records; administrative claims data; patient-generated data from fitness trackers or home medical equipment; disease registries; historical clinical trial data; and the like. By reducing or eliminating the need to enroll control participants, a synthetic control arm can increase efficiency, reduce delays, lower trial costs, and speed life-saving therapies to market. For instance, instead of having to recruit 1,000 patients, e.g., 500 for the active arm, and 500 for the control arm, only 500 participants need to be recruited when a synthetic control arm is employed.

The term “specialty drug” can include a drug that can be prescribed to treat a complex, chronic, rare, or difficult-to-manage disease or disorder. In some cases, such a disease can be cancer, an autoimmune disease, an inflammatory disorder, a chronic viral infection, a rare disease, and the like. A specialty drug can meet one or more of the following criteria: specialist-initiated (e.g., oncologist, rheumatologist); biotech drug (covers both IP protected drugs, generics and biosimilars); injectable formulation; costs more than $6,000 per year; requires special handling; limited distribution; necessitates risk evaluation and mitigation strategies (REMS) program. In some cases, a single dose or single course treatment of a specialty drug, e.g., gene therapy, can provide cure or disease remission; in such instances, per dose drug cost or per course drug cost is used for such purposes.

The terms “protein” can be used interchangeably to encompass both naturally-occurring and non-naturally occurring polypeptides, and fragments, mutants, derivatives and analogs thereof. A protein may be monomeric or polymeric. Further, a protein may comprise a number of different domains each of which has one or more distinct activities. In some cases, a protein can be at least 40 amino acids in length. A protein can comprise an overall charge based on pKa of side chains of component amino acids. In some instances, a protein can have an overall positive charge. In some instances, a protein can have an overall negative charge. In some instances, a protein can have an overall neutral charge. A protein can furthermore exist as a zwitterion. In some cases, the charge of the side chains may depend upon the local conditions of evaluation, e.g., pH of solution.

The term “recombinant” can refer to a biomolecule, e.g., a gene or protein, that (1) can be removed from its naturally occurring environment, (2) can be isolated from all or a portion of a polynucleotide in which the gene may be found in nature, (3) can be operatively linked to a polynucleotide which it may not be linked to in nature, or (4) may not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism can be recombinant, for example, if it is synthesized from an mRNA synthesized from a recombinant gene present in the cell.

The terms “administration,” “co-administration,” “administered in combination with” and their grammatical equivalents or the like, as used herein, can encompass administration of medicaments to a subject, and can include treatment regimens in which medicaments are administered by the same or different route of administration or at the same or different times. In some embodiments, a medicament can be co-administered with other agents (e.g., other medicaments). These terms can encompass administration of one or more medicaments to a subject so that medicaments and/or their metabolites are present in the subject at the same time. They can include simultaneous administration, administration at different times, and/or administration in a composition in which one or more medicaments are present. Thus, in some embodiments, a medicament(s) can be administered in a single composition. In some embodiments, a medicament(s) can be admixed in the composition. In some embodiments, the same medicament can be administered via a combination of different routes of administration. In some embodiments, medicament(s) administered can be in a therapeutically effective amount. In some embodiments, prodrugs may be converted into active forms.

The dates of regulatory milestones (in a particular jurisdiction; which may include submission dates, application dates, and approval dates, and the like) can uniquely identify a regulated product (after the fact). Combination of dates and features can provide unique identifiers for a regulated product. Dates, e.g., months, January, February, March, April, May, June, July, August, September, October, November, December, with days, e.g., 1 through 31, with years, e.g., 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, or 2044, can specify a regulatory milestone described above. A combination of these milestones (e.g., IND filing date, application for Phase-1, first patient dosing, completion of Phase-1, initiation of Phase-2, NDA filing date, regulatory approval date, and the like) can uniquely identify any product (past, present, or future).

In some cases, a regulated product may not have been approved or licensed by a regulatory agency, in the relevant jurisdiction, before 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, or 2100. In some cases, a medicament, drug, or drug candidate may not have had an investigational new drug (IND) application submitted to a regulatory agency before 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, or 2100. In some cases, a medicament, drug, or drug candidate may not have entered or completed a Phase 1, Phase 2, or Phase 3 clinical trial before 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, or 2100.

A “regulatory agency” can include a drug regulating authority can be responsible for enforcing rules and regulations, and issue guidelines for drug development, licensing, registration, manufacturing, marketing, and labeling of pharmaceutical products. Examples of regulatory agencies can include the US Food and Drug Administration (FDA); the UK Medicines and Healthcare Products Regulatory Agency (MHRA); the Australian Therapeutic Goods Administration (TGA); the Indian Central Drug Standard Control Organization (CDSCO); Health Canada; the European Medicines Agency (EMEA); the Danish Medicines Agency; the Costa Rican Ministry of Health; the New Zealand Medsafe—Medicines and Medical Devices Safety Authority; the Swedish Medical Products Agency (MPA); the Ministry of Public Health in Thailand; the Chinese State Food and Drug Administration; the German Federal Institute for Drugs and Medical Devices; the Malaysian National Pharmaceutical Control Bureau—Ministry of Health, Drugs Control Organization; Ministry of Health in Pakistan; the South African Medicines Control Council; the Sri Lankan SPC—Ministry of Health; The Swiss Agency for Therapeutic Products; the Agencia Nacional de Vigiloncia Sanitaria (ANVISA) in Brazil; the Japanese Ministry of Health, Labour & Welfare (MHLW); the World Health Organization; the Pan American Health Organization; the World Trade Organization; the International Conference on Harmonization; and the World Intellectual Property Organization.

General

This disclosure provides methods of treating subjects diagnosed with, or suspected of having, a disease or condition. The methods can include administering a subject a conventional treatment that can be a drug that has been licensed and approved by a regulatory agency to treat the disease or condition. Alternatively, the methods can include enrolling the subject in a clinical trial to receive a medicament that can be a non-licensed or non-approved drug or drug candidate; or in some cases a licensed and approved medicament that is not being marketed in the same territory as the territory that the clinical trial is conducted. Subjects enrolled in a clinical trial can be monitored throughout the trial using assays in order to determine efficacy of the medicament in the subject. In some cases, a medicament not showing efficacy in the subject can be swapped for another non-licensed or non-approved drug or drug candidate. In some cases, a method can comprise a comparing, e.g., biosimilar or small molecule generics as alternative to a reference biologic or reference small molecule, including a reference drug from Table 1, 2, or 3.

Products (e.g., medicaments, diagnostics, databases, and the like) from a clinical trial as described herein can be subject to regulatory clearance by a regulatory agency (e.g., the US FDA). In some cases, products from a clinical trial as described herein may not be subject to regulatory clearance by a regulatory agency.

Costs for participation in the clinical trial can be provided by a clinical trial sponsor. While conventional clinical trials can be sponsored by a party such as a drug manufacturer, it is envisaged that clinical trials disclosed here can be sponsored by a different party, e.g., who does not partially or substantially own or have licensed intellectual property to the medicament, its formulation, or its method of use. Furthermore, it is envisaged in these new trials that the third-party payer of the clinical trial may, in certain embodiments, only pay for participation in clinical trial on behalf of a subject enrolled in the clinical trial when treatment of the subject is successful (i.e., remission is achieved).

A third-party provider of the clinical trial can provide licensed and approved; or non-licensed or non-approved drugs or drug candidates for use in the clinical trial. Generally, a medicament that is a non-licensed or non-approved drug or drug candidate can be biosimilar or a generic equivalent to a drug licensed and approved by a regulatory agency to treat the disease or condition. Such a biosimilar can be produced, manufactured, or sold through an exemption to patent protection. A medicament subject to exemption to patent protection can be administered in order to provide clinical trial data for the medicament to a regulatory agency. Such data can be used by the provider to obtain regulatory approval for the medicament or formulations thereof.

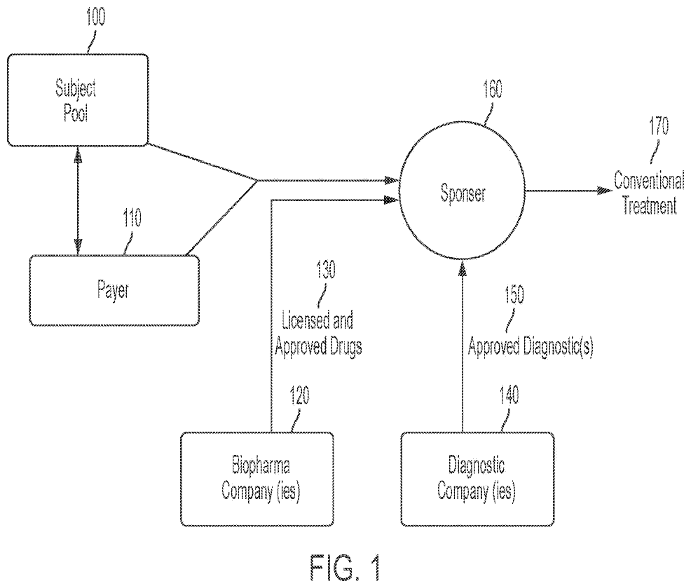

Referring to FIG. 1, one aspect of treatment can include a conventional treatment. A subject pool 100 harboring, previously diagnosed with, or suspected of having, a disease or condition can be referred to a provider 160 for treatment. Costs for the treatment can be provided by a third-party sponsor 110. A third-party sponsor 110 can be a public or private insurer; a government insurance or healthcare agency such as Medicare, Medicaid, Veterans Administration (VA), or a Home Health Agency; an employer group health plan, a pension fund, and the like. A provider 160 can be provided licensed and approved drugs 130 for administration to the subject pool 100. A diagnostic company 140 can provide diagnostics 150 to aid in properly distributing drugs 130 to subjects 100. A diagnostic 150 can include those that have been approved and or licensed by a regulatory agency, for example, as a companion diagnostic. Upon confirming a diagnosis, the provider 160 can facilitate a conventional treatment 170 that can include administering the licensed and approved drug 130 as indicated on the drug label.

In conducting the treatment paradigm depicted in FIG. 1, the sponsor 110 can provide payment for subjects 100 to receive the conventional treatment 170. Payment by the sponsor 110 can be provided to the provider 160, who can reimburse a biopharma company 120 or a diagnostic company 140 for use of drugs 130 or diagnostics 150, respectively, in the conventional treatment 170. The sponsor 110 in this paradigm provides payment whether or not the drug 130 shows efficacy when administered using the conventional treatment 170.

Referring to FIG. 2, another aspect can include either conventional treatment of administration of a medicament that can be biosimilar to a licensed and approved drug in a clinical trial. One embodiment can include the provider 160 receiving licensed and approved drugs 130 from one biopharma company 120, and receiving non-licensed or non-approved medicaments 230 that are biosimilar to the licensed and approved drug 130 from a biosimilar drug manufacturer 220. Further embodiments can include the provider 160 receiving both licensed diagnostics 150 and non-licensed diagnostics 250 from a diagnostic company 140. The subject pool 100 can undergo stratification 210 by the provider 160 to receive either the conventional treatment 170, or to be enrolled in a clinical trial 260 for administration 270 of the non-licensed or non-approved biosimilar medicament 230. In order to stratify the subject pool 100, the licensed diagnostic 150 or the non-licensed diagnostic 250 can be used. Data, such as efficacy and safety data, can be collected from administering the conventional treatment 170 or administering 270 the biosimilar 230 and stored in a data warehouse 280. Conversely, data from the data warehouse 280 that has been previously obtained can be used to guide either the administration of the biosimilar 230 or administration of the conventional treatment 170.

In conducting the treatment paradigm depicted in FIG. 2, the sponsor 110 can provide payment for subjects 100 to receive the conventional treatment 170. As discussed with respect to the paradigm depicted in FIG. 1, payment by the sponsor 110 can be provided to the provider 160 who can reimburse a biopharma company 120 or a diagnostic company 140 for use of drugs 130 or diagnostics 150, respectively, in the conventional treatment 170. When proceeding with the conventional treatment 170, the sponsor 110 provides payment whether or not the drug 130 shows efficacy when administered using the subject.

Alternatively, the third-party sponsor 110 can provide payment to the provider 160 for the subjects 100 to enroll in a clinical trial 260 for administration 270 of a non-licensed or non-approved medicament 230 that can be biosimilar to the licensed and approved drug 130. Under this treatment paradigm, the provider 160 may reimburse the sponsor 110 a portion of, or the entirety of, the costs paid by the third-party sponsor 110 if the administration 270 of the non-licensed and non-approved medicament 230 fails to show efficacy in the subject 100. Furthermore, a third-party sponsor 110 may only provide payment to a provider after the administration 270 of the non-licensed and non-approved medicament 230 shows efficacy in the subject 100, or for as long as the non-licensed and non-approved medicament 230 continues to show efficacy in the subject. It is envisaged that such payment to and/or reimbursement from the provider 160 can occur on a subject-by-subject basis.

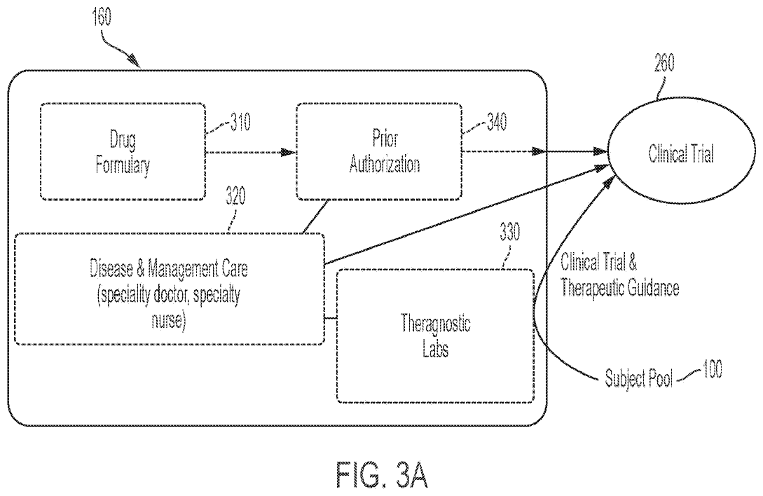

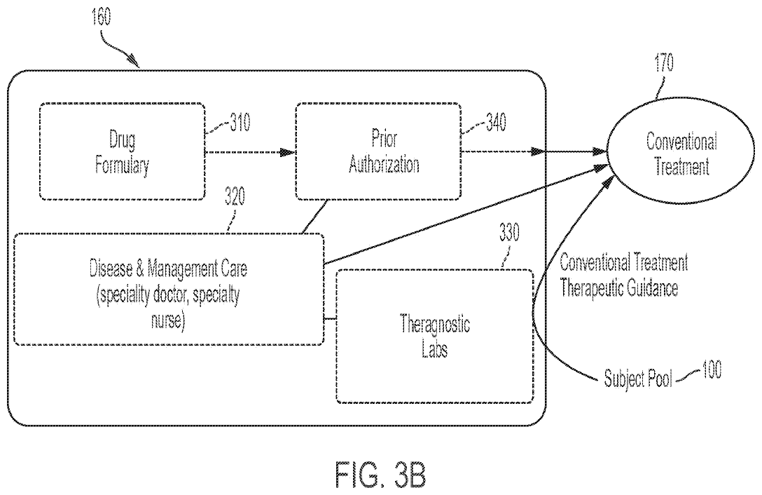

FIG. 3A and FIG. 3B depict exemplary components of the provider 160. The provider can employ, for example, a drug formulary 310, a disease and therapy management care team 320, and a theragnostic lab 330 to provide prior authorization 340 for the subjects 100 to enter a clinical trial 260 or receive a conventional treatment 170 as depicted in FIG. 2. Data in the form of clinical trial guidance and conventional treatment therapeutic guidance can be generated and stored on a database as described herein.

A drug formulary 310 can contain an electronic database of available therapeutics, or corresponding treatments using such, as well as a physical storage or pharmacy that stocks the treatments. A drug formulary 310 can have drugs 130 (depicted in FIG. 2) that are approved and licensed by a regulatory agency, as well as medicaments 230 that are not licensed or not approved by the regulatory agency.

A theragnostic lab 330 can be a facility capable of performing a diagnostic method to determine a specific disease state in the subject 100. The theragnostic lab 330 can employ licensed diagnostics 150 and non-licensed diagnostics 250 obtained from diagnostic companies 140, as well as diagnostic tests that can be developed in house. Diagnostic tests can include evaluation of biomarkers or assays for use in determining a disease state. In some cases, a diagnostic 150 can be licensed by a regulatory agency, for example, as a companion diagnostic. A diagnostic test can be a non-licensed diagnostic 250 that can become licensed by a regulatory agency after conducting a clinical trial.

A non-licensed diagnostic 250 can be a diagnostic that is not subject to clearance or approval by a regulatory agency (e.g., the USFDA). In some cases, a diagnostic can be a research tool that may not be subject to regulatory approval by a regulatory agency (e.g., the USFDA). Such a research tool can include determination of nucleic acid or protein sequence (e.g., next generation sequencing, solid phase sequencing, microarrays, and the like), whether direct or indirect, including, e.g., determination of gene copy numbers and allelic variants. In some cases, a diagnostic can include predictive algorithms (e.g., machine learning) that may not be subject to regulatory approval by a regulatory agency (e.g., the USFDA or equivalent counterparts).

A disease and therapy management care team 320 can comprise a group of healthcare professionals, such as a specialty doctor or a specialty nurse, that can interpret results obtained from the theragnostic lab 330 and other information, e.g., from medical records some of which may be included in a data warehouse 280. The disease and clinical management team 320 can access the drug formulary 310 and can seamlessly and efficiently provide a prior authorization 340 to receive a licensed and approved drug 130 or a non-licensed or non-approved medicament 230 based on the data obtained from the theragnostic lab 330 as well as the treatments available in the drug formulary 310. A prior authorization 340 can include allowing timely approval for expeditious payment for a subject 100 to receive a conventional treatment 170, allowing a subject to enter a clinical trial 260 to receive a non-licensed or non-approved medicament 230, or allowing the subject to choose between the two options.

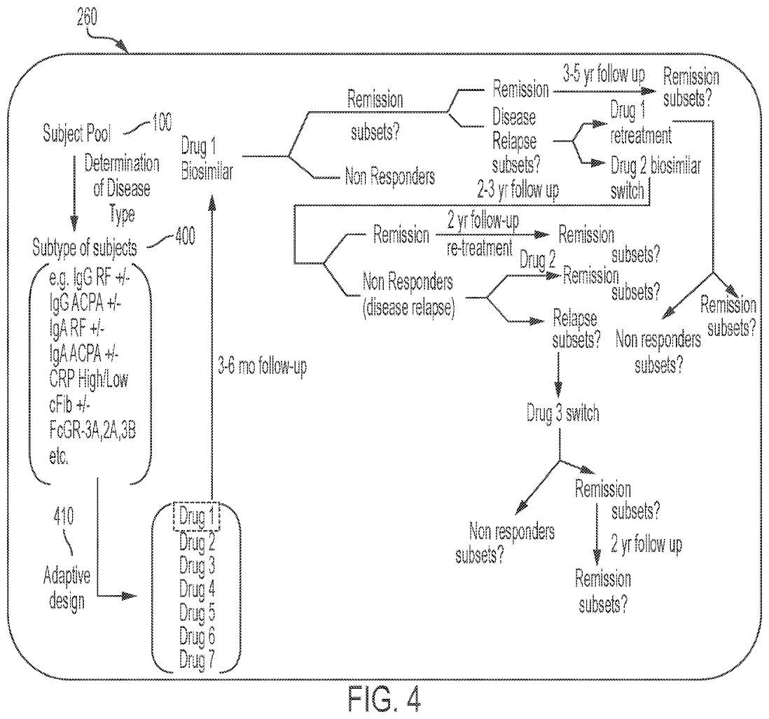

After a provider 160 provides prior authorization 340, a subject pool 100 can be expeditiously enrolled in a clinical trial 260. FIG. 4 depicts a subject pool 100 enrolled in the clinical trial 260. The disease type can be determined using licensed diagnostics 150 or non-licensed diagnostics 250 in order to produce subtypes of subjects 400 based on the disease subtype, here a Rheumatoid Arthritis (RA) example. Non-licensed or non-approved medicaments 230 that are biosimilar to approved and licensed drugs 130 can be administered to subjects based on projected efficacy against disease subtypes. Subjects who do not show remission after a fixed amount of time can be administered an alternative or additional therapies to accomplish maximal response. This may include additional drugs biosimilar to existing approved and licensed drugs 130. This treatment paradigm can continue until a subject achieves remission or fails to achieve remission.

Data from clinical trials 260 can be input into systems that can store and transmit the data. An exemplary embodiment is depicted in FIG. 5. Data 520 obtained from patient samples 510 obtained from subjects 100 in the clinical trial 260 can be input into clinical trial system 530 by a provider 160 of the clinical trial 260. The data 520 can be stored using storage medium 540. In some cases, storage medium 540 can be a component of a data warehouse 280 as depicted in FIG. 2 that can house clinical trial data. In some cases, the data obtained, for example, by conducting a clinical trial 260 using a medicament 230 that is non-licensed or non-approved by a regulatory agency can become licensed or approved by the regulatory agency through the clinical trial 260. The clinical trial data 520 can be used to support an application to a regulatory agency for approval of the medicament 230 based on the clinical trial data 520, which can be subject to exemptions to patent protection such as provisions outlined in 35 U.S.C. § 271(e) in the United States. In some cases, a medicament 230 seeking approval based on the clinical trial data 520 can have exemption (safe harbor) from infringing an existing patent through the following provision: “It shall not be an act of infringement to make, use, offer to sell, or sell within the United States or import into the United States a patented invention (other than a new animal drug or veterinary biological product (as those terms are used in the Federal Food, Drug, and Cosmetic Act and the Act of Mar. 4, 1913) which is primarily manufactured using recombinant DNA, recombinant RNA, hybridoma technology, or other processes involving site specific genetic manipulation techniques) solely for uses reasonably related to the development and submission of information under a Federal law which regulates the manufacture, use, or sale of drugs or veterinary biological products.” Patent protection for drugs developed in clinical trials can be pursued based on the clinical trial data and novel formulations that can be developed therein.

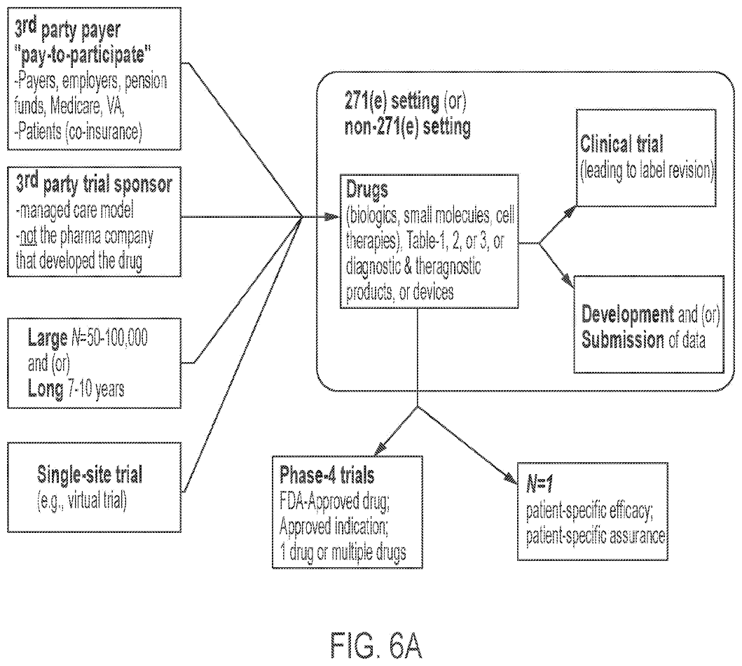

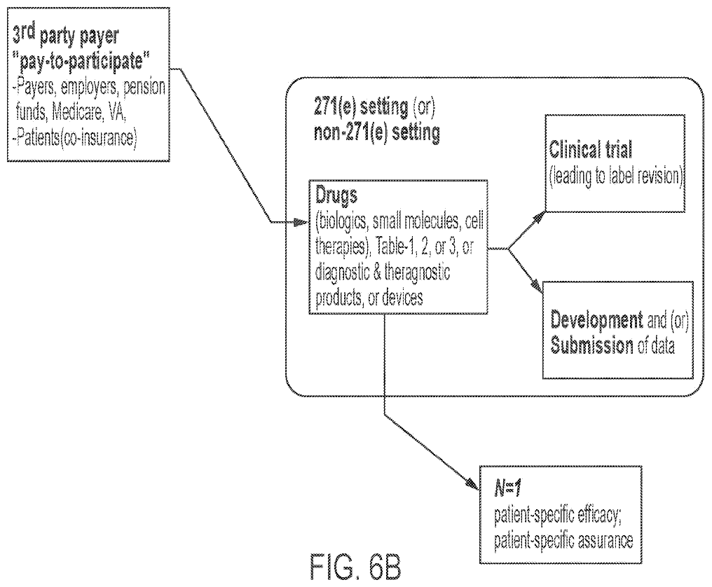

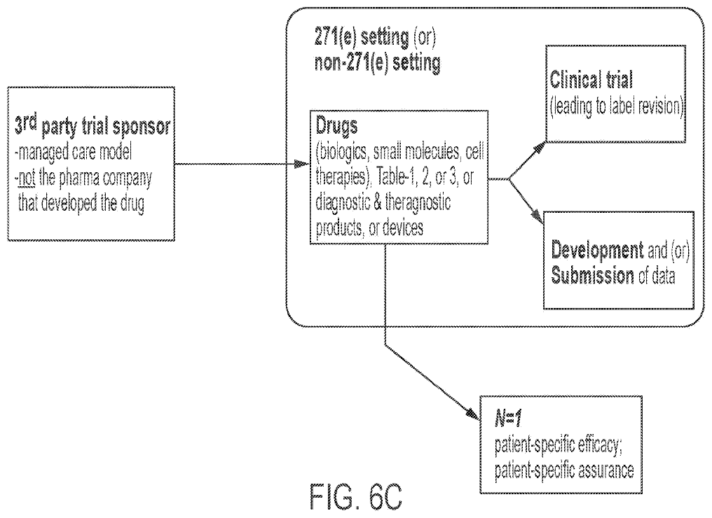

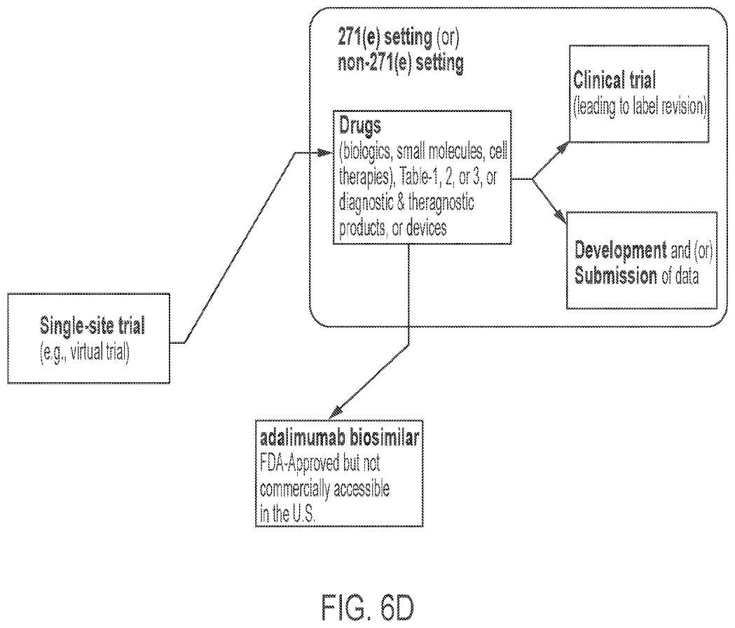

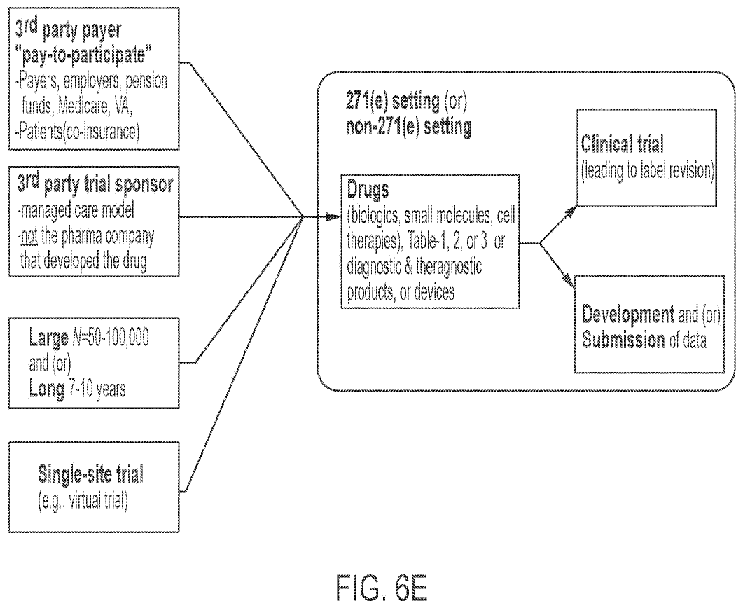

FIG. 6A-E depict exemplary features of conducting a sizeable clinical trial.

Referring to FIG. 6A, one aspect of the trial can have the exemption provision (from patent infringement) as codified in relevant part at 35 U.S.C. § 271(e), or where the exemption (safe harbor) provision is not required (non-271(e) setting). In conducting the clinical trial, one notable feature can include a “pay-to-participate” component, where the fee is paid directly or indirectly by an insurer, third-party payer, various healthcare providers (e.g., pension funds, Medicare, Veteran's Administration), or patient themselves; but distinguished from the traditional clinical trial sponsors who fund clinical trials, e.g., a pharmaceutical company, a government funding agency, e.g., NIH. Another feature, independently or combined with one or more other features, can include a third-party sponsor, e.g., a managed care company; but distinguished from the traditional clinical trial sponsors, e.g., a pharmaceutical company. Another feature, independently or combined with one or more other features, can be a sizeable trial size (e.g., greater than about 2000, 4000, 8000, 30000, etc.) and/or a sizeable trial duration (e.g., greater than about 2, 4, 6, 7, 8, 9, or 10 years). Other notable features, which can be added in various combinations, can include where the trial is performed either from a single clinical site or virtually; where the study is a phase-4 study; for a drug counterpart of a therapeutic selected from Table 1, 2, or 3; or where some assurance is provided to individual patients, e.g., N=1 efficacy and/or financial assurance).

Referring to FIG. 6B, one aspect of the trial can be a “pay-to-participate” trial, which can be testing a drug, biologic, small molecule, cell therapy, therapeutic selected from Table 1, 2, or 3, diagnostic, DNA or cell therapy, device, and the like. The pay-to-participate amount can cover some or all of the drug or therapeutic cost and (or) the services accompanying treatment costs, which can be funded by a third-party payer, as described in FIG. 6A. In some aspects N=1 patient-specific assurance can be provided, e.g., therapeutic and/or financial assurance.

Referring to FIG. 6C, one aspect of a clinical trial can be a third-party sponsor, e.g., a managed care entity, but distinguished from the classic sponsors, e.g., a pharmaceutical company. Drugs can be tested, including therapeutics selected from Table 1, 2, or 3. In some cases, patient-specific assurance can be provided, e.g., N=1 therapeutic and (or) financial assurances.

Referring to FIG. 6D, one aspect of the trial is that it is a single site trial, e.g., a virtual site. In some cases, this feature can be applied in a trial that can have the exemption (safe harbor) provision as codified in relevant part at 35 U.S.C. § 271(e), or where the exemption (safe harbor) provision is not required (non-271(e) setting). In certain cases, a drug, biologic, small molecule, cell therapy, therapeutic selected from Table 1, 2, or 3, diagnostic, DNA or cell therapy, medical device, and the like, is the subject of the trial. In certain cases, the therapeutic tested can be, e.g., an adalimumab biosimilar. In some cases, additional limitations can be added in various combinations, e.g., pay-to-participate features, third-party sponsor, sizeable trials, and (or) patient-specific assurance, e.g., N=1 therapeutic and (or) financial assurances.

Referring to FIG. 6E, one aspect of the trial is that it can be conducted from a single or virtual site. In certain cases, additional features can be added, e.g., where the study is a phase-4 study, and/or where some assurance is provided to individual patients, e.g., efficacy and/or financial assurance. In some cases, the trial can be directed to a drug, e.g., a biosimilar or generic that is equivalent to a reference drug from, e.g., Tables 1, 2, or 3.

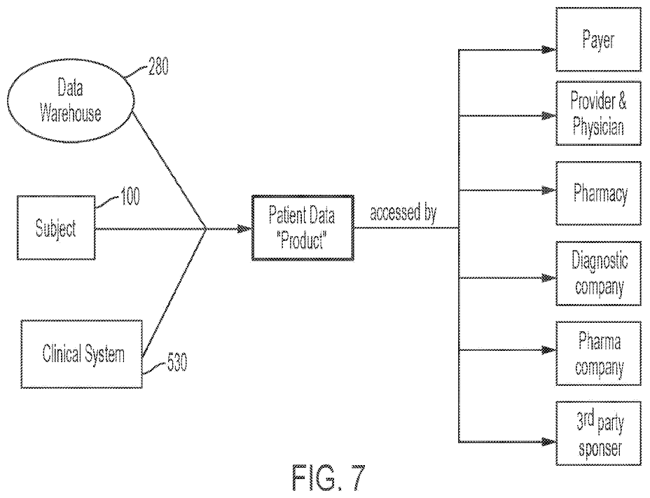

FIG. 7 shows subject-centric (or sample; or collections of samples or subjects), or subject data-centric (individual data, or disease-specific data, subset-specific data, etc.):

(A) Doctors, healthcare professionals, technicians, assistants, recordkeepers, administrators, receptionists, employees, agents, advisers, consultants, and the like, who have access to (or possess or generate) subject sample, subject, or data. Including those who archive data or samples.
(B) Sponsor, study planners, strategic or operational study planners, consultants, agents, advisers, assistants, statisticians, diagnostic and other technicians, documentation experts and handlers, facilitators who address permission, approval, subject informed consent, ethics review, all other components of study, compliance auditing and verification, and the like. In particular, those who evaluate and plan study, including, e.g., who to include and study, what questions will be addressed in the study, where locationwise the study will take place (which include one or more of the subject, subject sample, or data point above) whether in a local jurisdiction or including a subject, data point, or site in a different (e.g., foreign) jurisdiction, when data is collected (including over what period), what features and how they will be evaluated to collect data in the study. Individuals or entities directly or indirectly participating in running the study, or in its planning or data analysis would be thus included.
(C) Pharmacist, pharmacy, supplier, diagnostic laboratory, technicians, employees, adviser, agent, or others who actually supply or perform goods or services in support of the trial on the subject or collect the data. This can include the drug itself, and associated goods and services in its use, but also the diagnostic entities which evaluate the subject or samples, and the data which is then typically incorporated into a medical record of the subject. The data can include the control and standards for the diagnostic tests.
(D) Insurance entity, employees, consultants, agents, advisers, administrators who have access to the subject data. This data may be used, e.g., to determine coverage, as well as to determine population statistics on the subject, subject categories, subsets of like patients, on such features as response to treatment, costs of treatment, typical costs of treatment, and the like. These may be used by the insurer to determine future policy costs, projections on treatment efficiencies, different response categories, and the like. Also provided are systems which contain the data, the data points themselves, and uses thereof (see FIG. 7).

The strategies described herein are applicable to certain biologics, e.g., biosimilars, and to small molecule “generic” counterpart medicaments. Thus, the reference drugs may be biologics, or small molecule compounds or compositions. The methodologies may apply to other regulated articles, e.g., diagnostic articles and methods, therapeutic articles and methods, medical devices, other non-medical devices, external devices, implants, jigs associated with implanted or other devices, and the like. See, for example, 21 U.S.C. § 321(g) and (h), which describes a “drug” as (A) articles recognized in the official United States Pharmacopoeia, official Homoeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals; and (D) articles intended for use as a component of any article specified in clause (A), (B), or (C). Similarly, a device can mean an instrument, apparatus, implement, machine, contrivance, implant, in vitro reagent, or other similar or related article, including any component, part, or accessory, which is (a) recognized in the official National Formulary, or the United States Pharmacopeia, or any supplement to them; (b) intended for use in the diagnosis of disease or other conditions, or in the cure, mitigation, treatment, or prevention of disease, in man or other animals; or (c) intended to affect the structure or any function of the body of man or other animals, and which does not achieve its primary intended purposes through chemical action within or on the body of man or other animals and which is not dependent upon being metabolized for the achievement of its primary intended purposes. Thus, the scope of the relevant articles can include medicaments, articles for diagnosis, cure, mitigation, treatment, or prevention of disease, and articles which are components of such. An instrument, apparatus, implement, machine, contrivance, implant, in vitro reagent, or other similar or related article, including any component, part, or accessory can also be included. Diagnostic articles or components; cell- or tissue-based articles or components; gene therapy articles or components; vaccines, device articles or components, implants, transplants, prostheses, interfaces, jigs, and related components can also be included. The scope of articles regulated is not limited to medicaments, and covers not only the articles themselves, but the equipment and methods for manufacture, use, offer to sell, and/or sale of such, or import.

Historically, clinical trials have been categorized into, among others, prevention trials (e.g., how to prevent initially or recurrence of a condition), screening trials (e.g., detection of a condition), diagnostic trials (e.g., study or compare tests or procedures for diagnosing a condition), treatment trials (e.g., test new treatments, therapeutics, combinations of such or new approaches of medical intervention), behavioral trials, quality of life trials (e.g., explore and measure or evaluate ways to improve comfort and/or quality of life), and compassionate use trials (e.g., expanded access or last resort, where no alternative effective treatments have been developed). In addition, trial designs might be categorized into, among others, fixed trials (e.g., where participants enter or leave trial, according to fixed criteria set by design), adaptive trials (e.g., where data generated during the trial are used to design the trial and interim data is used to modify trial as it proceeds; may modify, e.g., dosage, sample sizes, drug (therapeutic), patient selection criteria; often apply a Bayesian experimental design to assess the trial's progress), and “complex innovative design” (CID; including the use of adaptive, Bayesian, and other novel statistical approaches; see, e.g., US FDA CID pilot program and CID webpage, and counterpart descriptions used by other regulatory agencies as examples and strategies being used in these trials).

Various features of clinical trials can include randomized (e.g., where participants are randomly assigned to various study arms), blinded (e.g., where participants do not know which of alternative treatments they receive), double blinded (e.g., where neither participants nor researchers know which of alternative treatments they receive), or double dummy (e.g., in alternating periods, with possible switching of (or between) treatments).

Important features of a clinical trial, e.g., a sizeable trial, are: informed consent, statistical power (e.g., sufficiently powered trial size), placebo groups, appropriate duration, and proper recordkeeping (e.g., proper clinical practices, often using Electronic Data Capture (EDC)).

Medicaments

In some cases, a medicament can be defined by molecular weight. In some cases, a medicament can have a molecular weight of from about 50 to about 2000 Da, from about 50 to about 1950 Da, from about 50 to about 1900 Da, from about 50 to about 1850 Da, from about 50 to about 1800 Da, from about 50 to about 1750 Da, from about 50 to about 1700 Da, from about 50 to about 1650 Da, from about 50 to about 1600 Da, from about 50 to about 1550 Da, from about 50 to about 1500 Da, from about 50 to about 1450 Da, from about 50 to about 1400 Da, from about 50 to about 1350 Da, from about 50 to about 1300 Da, from about 50 to about 1250 Da, from about 50 to about 1200 Da, from about 50 to about 1150 Da, from about 50 to about 1100 Da, from about 50 to about 1050 Da, from about 50 to about 1000 Da, from about 50 to about 950 Da, from about 50 to about 900 Da, from about 50 to about 850 Da, from about 50 to about 800 Da, from about 50 to about 750 Da, from about 50 to about 700 Da, from about 50 to about 650 Da, from about 50 to about 600 Da, from about 50 to about 550 Da, from about 50 to about 500 Da, from about 50 to about 450 Da, from about 50 to about 400 Da, from about 50 to about 350 Da, from about 50 to about 300 Da, from about 50 to about 250 Da, from about 50 to about 200 Da, from about 50 to about 150 Da, or from about 50 to about 100 Da. In some cases, a medicament can have a molecular weight of from about 40 to about 1000, from about 40 to about 990, from about 40 to about 980, from about 40 to about 970, from about 40 to about 960, from about 40 to about 950, from about 40 to about 940, from about 40 to about 930, from about 40 to about 920, from about 40 to about 910, from about 40 to about 900, from about 40 to about 890, from about 40 to about 880, from about 40 to about 870, from about 40 to about 860, from about 40 to about 850, from about 40 to about 840, from about 40 to about 830, from about 40 to about 820, from about 40 to about 810, from about 40 to about 800, from about 40 to about 790, from about 40 to about 780, from about 40 to about 770, from about 40 to about 760, from about 40 to about 750, from about 40 to about 740, from about 40 to about 730, from about 40 to about 720, from about 40 to about 710, from about 40 to about 700, from about 40 to about 690, from about 40 to about 680, from about 40 to about 670, from about 40 to about 660, from about 40 to about 650, from about 40 to about 640, from about 40 to about 630, from about 40 to about 620, from about 40 to about 610, from about 40 to about 600, from about 40 to about 590, from about 40 to about 580, from about 40 to about 570, from about 40 to about 560, from about 40 to about 550, from about 40 to about 540, from about 40 to about 530, from about 40 to about 520, from about 40 to about 510, from about 40 to about 500, from about 40 to about 490, from about 40 to about 480, from about 40 to about 470, from about 40 to about 460, from about 40 to about 450, from about 40 to about 440, from about 40 to about 430, from about 40 to about 420, from about 40 to about 410, or from about 40 to about 400 Da. In some cases, a medicament can have a MW of at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, or 200 Da. In some cases, a medicament can have a MW of at least about 100, 150, 200, 250, 300, 350, 400, 450, 500, 550, 600, 650, 700, 750, 800, 850, 900, 950, or 1000 Da. In some cases, a medicament can have a MW of at least about 0.5, 1, 1.5, 2, 2.5, 3, 3.5, 4, 4.5, 5, 5.5, 6, 6.5, 7, 7.5, 8, or 10 kDa. In some cases, a medicament can have a MW of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 kDa. In some cases, a medicament can have a MW of 100, 150, 200, 250, 300, 350, 400, 450, 500, 550, 600, 650, 700, 750, 800, 850, 900, 950, or 1000 kDa.

A medicament can include a biologic, such as a virus, therapeutic serum, a toxin, an antitoxin, a vaccine, blood, a blood component or derivative, an allergenic product, a non-chemically synthesized protein, or an analogous product, or arsphenamine or a derivative of arsphenamine, applicable to the prevention, treatment, or cure of a disease or condition of human beings. In some cases, a drug can include a protein, a living cell, a hormone, an immune cell, a blood cell, a clotting factor, a dermatologic toxin, a neurotoxin, a human or primate tissue, a monoclonal antibody or a fragment thereof, a polyclonal antibody or a fragment thereof, a recombinant microorganism, a probiotic, or a component of a human microbiota.

In some embodiments, a medicament can be a vaccine. In some embodiments, a medicament may not be a vaccine. In some embodiments, a medicament can be a therapeutic vaccine. In some embodiments, a medicament may not be a therapeutic vaccine. In some embodiments, a medicament can be a prophylactic vaccine. In some embodiments, a medicament may not be a prophylactic vaccine.

In some cases, a medicament can be a protein. A protein as used herein can include both naturally-occurring and non-naturally occurring polypeptides, as well as fragments, mutants, derivatives and analogs thereof. A protein may be monomeric or polymeric. Further, a protein may comprise a number of different domains each of which has one or more distinct activities. In some cases, a protein can be at least 40 amino acids in length. In some cases, a protein can be a therapeutic protein. A therapeutic protein can include an antithrombin, a fibrinolytic, an enzyme, an antineoplastic agent, a hormone, a fertility agent, an immunosuppressive agent, a bone factor, an antidiabetic agent, an antibody, or any combination thereof. In some cases, a protein can be an antithrombin. Examples of antithrombins can include lepirudin (LTYTDCTESGQNLCLCEGSNVCGQGNKCILGSDGEKNQCVTGEGTPKPQSHNDGDFEEIPE EYLQ), bivalirudin (FPRPGGGGNGDFEEIPEEYL), defibrotide, and sulodexide. In some cases, a protein can be a fibrinolytic. Examples of fibrinolytics can include lepirudin, reteplase (SYQGNSDCYFGNGSAYRGTHSLTESGASCLPWNSMILIGKVYTAQNPSAQALGLGKHNYC RNPDGDAKPWCHVLKNRRLTWEYCDVPSCSTCGLRQYSQPQFRIKGGLFADIASHPWQAAI FAKHRRSPGERFLCGGILISSCWILSAAHCFQERFPPHHLTVILGRTYRVVPGEEEQKFEVEK YIVHKEFDDDTYDNDIALLQLKSDSSRCAQESSVVRTVCLPPADLQLPDWTECELSGYGKH EALSPFYSERLKEAHVRLYPSSRCTSQHLLNRTVTDNMLCAGDTRSGGPQANLHDACQGDS GGPLVCLNDGRMTLVGIISWGLGCGQKDVPGVYTKVTNYLDWIRDNMRP), anistreplase (SYQVICRDEKTQMIYQQHQSWLRPVLRSNRVEYCWCNSGRAQCHSVPVKSCSEPRCFNGG TCQQALYFSDFVCQCPEGFAGKCCEIDTRATCYEDQGISYRGTWSTAESGAECTNWNSSAL AQKPYSGRRPDAIRLGLGNHNYCRNPDRDSKPWCYVFKAGKYSSEFCSTPACSEGNSDCYF GNGSAYRGTHSLTESGASCLPWNSMILIGKVYTAQNPSAQALGLGKHNYCRNPDGDAKPW CHVLKNRRLTWEYCDVPSCSTCGLRQYSQPQFRIKGGLFADIASHPWQAAIFAKHRRSPGE RFLCGGILISSCWILSAAHCFQERFPPHHLTVILGRTYRVVPGEEEQKFEVEKYIVHKEFDDD TYDNDIALLQLKSDSSRCAQESSVVRTVCLPPADLQLPDWTECELSGYGKHEALSPFYSERL KEAHVRLYPSSRCTSQHLLNRTVTDNMLCAGDTRSGGPQANLHDACQGDSGGPLVCLNDG RMTLVGIISWGLGCGQKDVPGVYTKVTNYLDWIRDNMRP), tenecteplase (SYQVICRDEKTQMIYQQHQSWLRPVLRSNRVEYCWCNSGRAQCHSVPVKSCSEPRCFNGG TCQQALYFSDFVCQCPEGFAGKCCEIDTRATCYEDQGISYRGNWSTAESGAECTNWQSSAL AQKPYSGRRPDAIRLGLGNHNYCRNPDRDSKPWCYVFKAGKYSSEFCSTPACSEGNSDCYF GNGSAYRGTHSLTESGASCLPWNSMILIGKVYTAQNPSAQALGLGKHNYCRNPDGDAKPW CHVLKNRRLTWEYCDVPSCSTCGLRQYSQPQFRIKGGLFADIASHPWQAAIFAAAAASPGE RFLCGGILISSCWILSAAHCFQERFPPHHLTVILGRTYRVVPGEEEQKFEVEKYIVHKEFDDD TYDNDIALLQLKSDSSRCAQESSVVRTVCLPPADLQLPDWTECELSGYGKHEALSPFYSERL KEAHVRLYPSSRCTSQHLLNRTVTDNMLCAGDTRSGGPQANLHDACQGDSGGPLVCLNDG RMTLVGIISWGLGCGQKDVPGVYTKVTNYLDWIRDNMRP), streptokinase, and sulodexide. In some cases, a protein can be an enzyme. Examples of enzymes can include dornase alfa (LKIAAFNIQTFGETKMSNATLVSYIVQILSRYDIALVQEVRDSHLTAVGKLLDNLNQDAPDT YHYVVSEPLGRNSYKERYLFVYRPDQVSAVDSYYYDDGCEPCGNDTFNREPAIVRFFSRFT EVREFAIVPLHAAPGDAVAEIDALYDVYLDVQEKWGLEDVMLMGDFNAGCSYVRPSQWS SIRLWTSPTFQWLIPDSADTTATPTHCAYDRIVVAGMLLRGAVVPDSALPFNFQAAYGLSD QLAQAISDHYPVEVMLK), velaglucerase alfa (ARPCIPKSFGYSSVVCVCNATYCDSFDPPTFPALGTFSRYESTRSGRRMELSMGPIQANHTG TGLLLTLQPEQKFQKVKGFGGAMTDAAALNILALSPPAQNLLLKSYFSEEGIGYNIIRVPMA SCDFSIRTYTYADTPDDFQLHNFSLPEEDTKLKIPLIHRALQLAQRPVSLLASPWTSPTWLKT NGAVNGKGSLKGQPGDIYHQTWARYFVKFLDAYAEHKLQFWAVTAENEPSAGLLSGYPF QCLGFTPEHQRDFIARDLGPTLANSTHEINVRLLMLDDQRLLLPHWAKVVLTDPEAAKYVH GIAVHWYLDFLAPAKATLGETHRLFPNTMLFASEACVGSKFWEQSVRLGSWDRGMQYSHS IITNLLYHVVGWTDWNLALNPEGGPNWVRNFVDSPIIVDITKDTFYKQPMFYHLGHFSKFIP EGSQRVGLVASQKNDLDAVALMHPDGSAVVVVLNRSSKDVPLTIKDPAVGFLETISPGYSI HTYLWRRQ, taliglucerase alfa (EFARPCIPKSFGYSSVVCVCNATYCDSFDPPTFPALGTFSRYESTRSGRRMELSMGPIQANH TGTGLLLTLQPEQKFQKVKGFGGAMTDAAALNILALSPPAQNLLLKSYFSEEGIGYNIIRVP MASCDFSIRTYTYADTPDDFQLHNFSLPEEDTKLKIPLIHRALQLAQRPVSLLASPWTSPTWL KTNGAVNGKGSLKGQPGDIYHQTWARYFVKFLDAYAEHKLQFWAVTAENEPSAGLLSGY PFQCLGFTPEHQRDFIARDLGPTLANSTHEINVRLLMLDDQRLLLPHWAKVVLTDPEAAKY VHGIAVHWYLDFLAPAKATLGETHRLFPNTMLFASEACVGSKFWEQSVRLGSWDRGMQY SHSIITNLLYHVVGWTDWNLALNPEGGPNWVRNFVDSPIIVDITKDTFYKQPMFYHLGHFSK FIPEGSQRVGLVASQKNDLDAVALMHPDGSAVVVVLNRSSKDVPLTIKDPAVGFLETISPGY SIHTYLWHRQDLLVDTM), asparaginase Erwinia chrysanthemi (ADKLPNIVILATGGTIAGSAATGTQTTGYKAGALGVDTLINAVPEVKKLANVKGEQFSNM ASENIVITGDVVLKLSQRVNELLARDDVDGVVITHGTDTVEESAYFLHLTVKSDKPVVFVAA MRPATAISADGPMNLLEAVRVAGDKQSRGRGVMVVLNDRIGSARYITKTNASTLDTFKAN EEGYLGVIIGNRIYYQNRIDKLHTTRSVFDVRGLTSLPKVDILYGYQDDPEYLYDAAIQHGV KGIVYAGMGAGSVSVRGIAGMRKAMEKGVVVIRSTRTGNGIVPPDEELPGLVSDSLNPAHA RILLMLALTRTSDPKVIQEYFHTY), glucarpidase (ALAQKRDNVLFQAATDEQPAVIKTLEKLVNIETGTGDAEGIAAAGNFLEAELKNLGFTVTR SKSAGLVVGDNIVGKIKGRGGKNLLLMSHMDTVYLKGILAKAPFRVEGDKAYGPGIADDK GGNAVILHTLKLLKEYGVRDYGTITVLFNTDEEKGSFGSRDLIQEEAKLADYVLSFEPTSAG DEKLSLGTSGIAYVQVNITGKASHAGAAPELGVNALVEASDLVLRTMNIDDKAKNLRFNW TIAKAGNVSNIIPASATLNADVRYARNEDFDAAMKTLEERAQQKKLPEADVKVIVTRGRPA FNAGEGGKKLVDKAVAYYKEAGGTLGVEERTGGGTDAAYAALSGKPVIESLGLPGFGYHS DKAEYVDISAIPRRLYMAARLIMDLGAGK), asfotase alfa (LVPEKEKDPKYWRDQAQETLKYALELQKLNTNVAKNVIMFLGDGMGVSTVTAARILKGQ LHHNPGEETRLEMDKFPFVALSKTYNTNAQVPDSAGTATAYLCGVKANEGTVGVSAATER SRCNTTQGNEVTSILRWAKDAGKSVGIVTTTRVNHATPSAAYAHSADRDWYSDNEMPPEA LSQGCKDIAYQLMHNIRDIDVIMGGGRKYMYPKNKTDVEYESDEKARGTRLDGLDLVDTW KSFKPRYKHSHFIWNRTELLTLDPHNVDYLLGLFEPGDMQYELNRNNVTDPSLSEMVVVAI QILRKNPKGFFLLVEGGRIDHGHHEGKAKQALHEAVEMDRAIGQAGSLTSSEDTLTVVTAD HSHVFTFGGYTPRGNSIFGLAPMLSDTDKKPFTAILYGNGPGYKVVGGERENVSMVDYAH NNYQAQSAVPLRHETHGGEDVAVFSKGPMAHLLHGVHEQNYVPHVMAYAACIGANLGH CAPASSLKDKTHTCPPCPAPELLGGPSVFLFPPKPKDTLMISRTPEVTCVVVDVSHEDPEVKF NWYVDGVEVHNAKTKPREEQYNSTYRVVSVLTVLHQDWLNGKEYKCKVSNKALPAPIEK TISKAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGKDIDDDDDDD DDD), elosulfase alfa (APQPPNILLLLMDDMGWGDLGVYGEPSRETPLCSPSRAALLTGRLPIRNGFYTTNAHARNL LKKAGYVSKIVGKWHLGHRPQFHPLKHGFNIPVYRDWEMVGRYYEEFPINLKTGEANLTFL YWAVDATHAPVYASKPFLGTSQRGRYGDVADNTFVFFTSDNGAALISAPEQGGSNGPFPG HVTAGQVSHQLGSIMDLFTTSLALAGLTLMDRPIFYYRGDTLMAATLGQHKAHFWTWTVT THNLEDHTKLPLIFHLGRDPGERFPLSFEALVPAQPQLNVCNWAVMNWAPPGCEKLGKPNL DRMAAEGLLFPNFYSANAYTPQEIVGGIPDSEQLLPEDEWFGSPNCHFGPYDNKARPQIYLQ EALDFIKRQARHHPFAVREIDDSIGKILELLQDLHLCGKQTTFEGGMREPALAWWPPSDRAI DGLNLLPTLLQGRNSWENFRQGIDFCPGQNVSGASAEYQEALSRITSVVQQHQCLTPPESIP KKCLWSH), sebelipase (SGGKLTAVDPETNMNVSEIISYWGFPSEEYLVETEDGYILCLNRIPHGRKNHSDKGPKPVVF LQHGLLADSSNWVTNLANSSLGFILADAGFDVWMGNSRGNTWSRKHKTLSVSQDEFWAF SYDEMAKYDLPASINFILNKTGQEQVYYVGHSQGTTIGFIAFSQIPELAKRIKMFFALGPVAS VAFCTSPMAKLGRLPDHLIKDLFGDKEFLPQSAFLKWLGTHVCTHVILKELCGNLCFLLCGF NERNLNMSRVDVYTTHSPAGTSVQNMLHWSQAVKFQKFQAFDWGSSAKNYFHYNQSYPP TYNVKDMLVPTAVWSGGHDWLADVYDVNILLTQITNLVFHESIPEWEHLDFIWGLDAPWR LYNKIINLMRKYQ), sarcosidase (SMTNETSDRPLVHFTPNKGWMNDPNGLWYDEKDAKWHLYFQYNPNDTVWGTPLFWGH ATSDDLTNWEDQPIAIAPKRNDSGAFSGSMVVDYNNTSGFFNDTIDPRQRCVAIWTYNTPES EEQYISYSLDGGYTFTEYQKNPVLAANSTQFRDPKVFWYEPSQKWIMTAAKSQDYKIEIYSS DDLKSWKLESAFANEGFLGYQYECPGLIEVPTEQDPSKSYWVMFISINPGAPAGGSFNQYFV GSFNGTHFEAFDNQSRVVDFGKDYYALQTFFNTDPTYGSALGIAWASNWEYSAFVPTNPW RSSMSLVRKFSLNTEYQANPETELINLKAEPILNISNAGPWSRFATNTTLTKANSYNVDLSNS TGTLEFELVYAVNTTQTISKSVFADLSLWFKGLEDPEEYLRMGFEVSASSFFLDRGNSKVKF VKENPYFTNRMSVNNQPFKSENDLSYYKVYGLLDQNILELYFNDGDVVSTNTYFMTTGNA LGSVNMTTGVDNLFYIDKFQVREVK), and pegloticase (TYKKNDEVEFVRTGYGKDMIKVLHIQRDGKYHSIKEVATTVQLTLSSKKDYLHGDNSDVI PTDTIKNTVNVLAKFKGIKSIETFAVTICEHFLSSFKHVIRAQVYVEEVPWKRFEKNGVKHV HAFIYTPTGTHFCEVEQIRNGPPVIHSGIKDLKVLKTTQSGFEGFIKDQFTTLPEVKDRCFATQ VYCKWRYHQGRDVDFEATWDTVRSIVLQKFAGPYDKGEYSPSVQKTLYDIQVLTLGQVPE IEDMEISLPNIHYLNIDMSKMGLINKEEVLLPLDNPYGKITGTVKRKLSSRL). In some cases, a protein can be an antineoplastic agent. Examples of anti-neoplastic agents can include cetuximab QVQLKQSGPGLVQPSQSLSITCTVSGFSLTNYGVHWVRQSPGKGLEWLGVIWSGGNTDYN TPFTSRLSINKDNSKSQVFFKMNSLQSNDTAIYYCARALTYYDYEFAYWGQGTLVTVSAAS TKGPSVFPLAPSSKSTSGGTAALGCLVKDYFPEPVTVSWNSGALTSGVHTFPAVLQSSGLYS LSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAKGQPREPQVYTLPPSRDELTKNQVSL TCLVKGFYPSDIAVEWESNGQPENNYKTTPPVLDSDGSFFLYSKLTVDKSRWQQGNVFSCS VMHEALHNHYTQKSLSLSPGK), denileukin diftitox (MGADDVVDSSKSFVMENFSSYHGTKPGYVDSIQKGIQKPKSGTQGNYDDDWKGFYSTDN KYDAAGYSVDNENPLSGKAGGVVKVTYPGLTKVLALKVDNAETIKKELGLSLTEPLMEQV GTEEFIKRFGDGASRVVLSLPFAEGSSSVEYINNWEQAKALSVELEINFETRGKRGQDAMYE YMAQACAGNRVRRSVGSSLSCINLDWDVIRDKTKTKIESLKEHGPIKNKMSESPNKTVSEE KAKQYLEEFHQTALEHPELSELKTVTGTNPVFAGANYAAWAVNVAQVIDSETADNLEKTT AALSILPGIGSVMGIADGAVHHNTEEIVAQSIALSSLMVAQAIPLVGELVDIGFAAYNFVESII NLFQVVHNSYNRPAYSPGHKTHAPTSSSTKKTQLQLEHLLLDLQMILNGINNYKNPKLTRM LTFKFYMPKKATELKHLQCLEEELKPLEEVLNLAQSKNFHLRPRDLISNINVIVLELKGSETT FMCEYADETATIVEFLNRWITFCQSIISTLT), leuprolide (PGlu-his-trp-ser-tyr-D-leu-leu-arg-pro-nhet), asparaginase, aldesleukin (MAPTSSSTKKTQLQLEHLLLDLQMILNGINNYKNPKLTRMLTFKFYMPKKATELKHLQCLE EELKPLEEVLNLAQSKNFHLRPRDLISNINVIVLELKGSETTFMCEYADETATIVEFLNRWITF CQSIISTLT), pegaspargase, interferon beta-1a, trastuzumab (DIQMTQSPSSLSASVGDRVTITCRASQDVNTAVAWYQQKPGKAPKLLIYSASFLYSGVPSR FSGSRSGTDFTLTISSLQPEDFATYYCQQHYTTPPTFGQGTKVEIKRTVAAPSVFIFPPSDEQL KSGTASVVCLLNNFYPREAKVQWKVDNALQSGNSQESVTEQDSKDSTYSLSSTLTLSKADY EKHKVYACEVTHQGLSSPVTKSFNRGEC), rituximab (QVQLQQPGAELVKPGASVKMSCKASGYTFTSYNMHWVKQTPGRGLEWIGAIYPGNGDTS YNQKFKGKATLTADKSSSTAYMQLSSLTSEDSAVYYCARSTYYGGDWYFNVWGAGTTVT VSAASTKGPSVFPLAPSSKSTSGGTAALGCLVKDYFPEPVTVSWNSGALTSGVHTFPAVLQS SGLYSLSSVVTVPSSSLGTQTYICNVNHKPSNTKVDKKAEPKSCDKTHTCPPCPAPELLGGPS VFLFPPKPKDTLMISRTPEVTCVVVDVSHEDPEVKFNWYVDGVEVHNAKTKPREEQYNSTY RVVSVLTVLHQDWLNGKEYKCKVSNKALPAPIEKTISKAKGQPREPQVYTLPPSRDELTKN QVSLTCLVKGFYPSDIAVEWESNGQPENNYKTTPPVLDSDGSFFLYSKLTVDKSRWQQGNV FSCSVMHEALHNHYTQKSLSLSPGK), ipilimumab (QVQLVESGGGVVQPGRSLRLSCAASGFTFSSYTMHWVRQAPGKGLEWVTFISYDGNNKY YADSVKGRFTISRDNSKNTLYLQMNSLRAEDTAIYYCARTGWLGPFDYWGQGTLVTVSSA STKGPSVFPLAPSSKSTSGGTAALGCLVKDYFPEPVTVSWNSGALTSGVHTFPAVLQSSGLY SLSSVVTVPSSSLGTQTYICNVNHKPSNTKVDKRVEPKSCDKTHTCPPCPAPELLGGPSVFLF PPKPKDTLMISRTPEVTCVVVDVSHEDPEVKFNWYVDGVEVHNAKTKPREEQYNSTYRVV SVLTVLHQDWLNGKEYKCKVSNKALPAPIEKTISKAKGQPREPQVYTLPPSRDELTKNQVS LTCLVKGFYPSDIAVEWESNGQPENNYKTTPPVLDSDGSFFLYSKLTVDKSRWQQGNVFSC SVMHEALHNHYTQKSLSLSPGK), aflibercept (SDTGRPFVEMYSEIPEIIHMTEGRELVIPCRVTSPNITVTLKKFPLDTLIPDGKRIIWDSRKGFI ISNATYKEIGLLTCEATVNGHLYKTNYLTHRQTNTIIDVVLSPSHGIELSVGEKLVLNCTART ELNVGIDFNWEYPSSKHQHKKLVNRDLKTQSGSEMKKFLSTLTIDGVTRSDQGLYTCAASS GLMTKKNSTFVRVHEKDKTHTCPPCPAPELLGGPSVFLFPPKPKDTLMISRTPEVTCVVVDV SHEDPEVKFNWYVDGVEVHNAKTKPREEQYNSTYRVVSVLTVLHQDWLNGKEYKCKVSN KALPAPIEKTISKAKGQPREPQVYTLPPSRDELTKNQVSLTCLVKGFYPSDIAVEWESNGQPE NNYKTTPPVLDSDGSFFLYSKLTVDKSRWQQGNVFSCSVMHEALHNHYTQKSLSLSPG), obinutuzumab, gemtuzumab ozogamicin, blinatumobab (DIQLTQSPASLAVSLGQRATISCKASQSVDYDGDSYLNWYQQIPGQPPKLLIYDASNLVSGI PPRFSGSGSGTDFTLNIHPVEKVDAATYHCQQSTEDPWTFGGGTKLEIKGGGGSGGGGSGG GGSQVQLQQSGAELVRPGSSVKISCKASGYAFSSYWMNWVKQRPGQGLEWIGQIWPGDG DTNYNGKFKGKATLTADESSSTAYMQLSSLASEDSAVYFCARRETTTVGRYYYAMDYWG QGTTVTVSSGGGGSDIKLQQSGAELARPGASVKMSCKTSGYTFTRYTMHWVKQRPGQGLE WIGYINPSRGYTNYNQKFKDKATLTTDKSSSTAYMQLSSLTSEDSAVYYCARYYDDHYCL DYWGQGTTLTVSSVEGGSGGSGGSGGSGGVDDIQLTQSPAIMSASPGEKVTMTCRASSSVS YMNWYQQKSGTSPKRWIYDTSKVASGVPYRFSGSGSGTSYSLTISSMEAEDAATYYCQQW SSNPLTFGAGTKLELKHRHHHH), daratumumab, vedolizumab, ustekinumab, siltuximab (EVQLVESGGKLLKPGGSLKLSCAASGFTFSSFAMSWFRQSPEKRLEWVAEISSGGSYTYYP DTVTGRFTISRDNAKNTLYLEMSSLRSEDTAMYYCARGLWGYYALDYWGQGTSVTVSSAS TKGPSVFPLAPSSKSTSGGTAALGCLVKDYFPEPVTVSWNSGALTSGVHTFPAVLQSSGLYS LSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAKGQPREPQVYTLPPSRDELTKNQVSL TCLVKGFYPSDIAVEWESNGQPENNYKTTPPVLDSDGSFFLYSKLTVDKSRWQQGNVFSCS VMHEALHNHYTQKSLSLSPGK), ramucirumab, pembrolizumab, ofatumumab, nivolumab, mepolizumab, brodalumab, canakinumab (QVQLVESGGGVVQPGRSLRLSCAASGFTFSVYGMNWVRQAPGKGLEWVAIIWYDGDNQ YYADSVKGRFTISRDNSKNTLYLQMNGLRAEDTAVYYCARDLRTGPFDYWGQGTLVTVSS ASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKRVEPKSCDKTHTCPPCPAPELLGGPSVFL FPPKPKDTLMISRTPEVTCVVVDVSHEDPEVKFNWYVDGVEVHNAKTKPREEQYNSTYRV VSVLTVLHQDWLNGKEYKCKVSNKALPAPIEKTISKAKGQPREPQVYTLPPSREEMTKNQV SLTCLVKGFYPSDIAVEWESNGQPENNYKTTPPVLDSDGSFFLYSKLTVDKSRWQQGNVFS CSVMHEALHNHYTQKSLSLSPGK), dinutuximab, lenograstim, and sipuleucel-T. In some cases, a protein can be a hormone. Examples of hormones can include cosyntropin, chorionic gonadotropin, and somatropin. In some cases, a protein can be a fertility agent. Examples of fertility agents can include leuprolide, menotropin, lutropin alfa, follitropin beta, urofollitropin, and choriogonadotropin alfa. In some cases, a protein can be an immunosuppressive agent. Examples of immunosuppressive agents can include etanercept, peginterferon alfa-2a, interferon alfa-n3, pegfilgrastim, sargramostim, peginterferon alfa-2b, anakinra, intravenous immunoglobulin, interferon gamma-1b, adalimumab, interferon beta-1a, infliximab, interferon beta-1b, interferon alfacon-1, basiliximab, muromonab, efalizumab, antithymocyte globulin, filgrastim, interferon alfa-2b, daclizumab, abatacept, rilocept, belatacept, natalizumab, blinatumomab, immune globulin, and ustekinumab. In some cases, a protein can be a bone factor. Examples of bone factors can include salmon calcitonin. In some cases, a protein can be an antidiabetic agent. Examples of antidiabetic agents can include insulin, insulin lispro, insulin glargine, insulin aspart, insulin detemir, insulin glulisine, and insulin isophane. In some cases, a protein can be an antibody. The term “antibody,” as used herein, can refer to immunoglobulin molecules and immunologically active portions of immunoglobulin molecules. In some cases, an immunologically active portion can be a portion that contain an antigen binding site that can immunospecifically bind an antigen. An immunoglobulin molecule can be of any type (e.g., IgG, IgE, IgM, IgD, IgA and IgY), class (e.g., IgG1, IgG2, IgG3, IgG4, IgA1 and IgA2) or subclass of immunoglobulin molecule. The terms “antibody” (Ab) or “monoclonal antibody” (mAb) can include intact molecules, antibody fragments (such as, for example, Fab, F(ab′)₂ fragments, or single chain fv fragments) which can be capable of specifically binding to a protein. Examples of antibodies can include canakinumab, ipilimumab, pertuzumab, denosumab, belimumab, raxibacumab, blinatumomab, anti-thymocyte globulin, dinutuximab, human varicella-zoster immune globulin, and ibritumomab tiuxetan.

In some cases, a medicament can be a living cell. A living cell can be a probiotic. Examples of probiotics can include Bacteroides caccae, Bacteroides capillosus, Bacteroides coagulans, Bacteroides distasonis, Bacteroides eggerthii, Bacteroides forsythus, Bacteroides fragilis, Bacteroides fragilis-ryhm, Bacteroides gracilis, Bacteroides levii, Bacteroides macacae, Bacteroides merdae, Bacteroides ovatus, Bacteroides pneumosintes, Bacteroides putredinis, Bacteroides pyogenes, Bacteroides splanchnicus, Bacteroides stercoris, Bacteroides tectum, Bacteroides thetaiotaomicron, Bacteroides uniformis, Bacteroides ureolyticus, Bacteroides vulgatus, Bacteroides fragilis ss. Vulgatus, Eubacterium aerofaciens, Bacteroides fragilis ss. Thetaiotaomicron, Blautia producta (previously known as Peptostreptococcus productus II), Bacteroides fragilis ss. Distasonis, Fusobacterium prausnitzii, Coprococcus eutactus, Eubacterium aerofaciens III, Blautia producta (previously known as Peptostreptococcus productus I), Ruminococcus bronii, Bifidobacterium adolescentis, Gemmiger formicilis, Bifidobacterium longum, Eubacterium siraeum, Ruminococcus torques, Eubacterium rectale Eubacterium rectale IV, Eubacterium eligens, Bacteroides eggerthii, Clostridium leptum, Bacteroides fragilis ss. A, Eubacterium biforme, Bifidobacterium infantis, Eubacterium rectale Coprococcus comes, Bacteroides capillosus, Ruminococcus albus, Eubacterium formicigenerans, Eubacterium haffii, Eubacterium ventriosum I, Fusobacterium russii, Ruminococcus obeum, Eubacterium rectale II, Clostridium ramosum I, Lactobacillus leichmanii, Ruminococcus cailidus, Butyrivibrio crossotus, Acidaminococcus fermentans, Eubacterium ventriosum, Bacteroides fragilis ss. fragilis, Bacteroides AR, Coprococcus catus, Eubacterium hadrum, Eubacterium cylindroides, Eubacterium ruminantium, Eubacterium CH-1, Staphylococcus epidermidis, Peptostreptococcus BL, Eubacterium limosum, Bacteroides praeacutus, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Eubacterium AG, -AK, -AL, -AL-1, -AN; Bacteroides fragilis ss. ovatus, -ss. d, -ss. f; Bacteroides L-1, L-5; Fusobacterium nucleatum, Fusobacterium mortiferum, Escherichia coli, Streptococcus morbiliorum, Peptococcus magnus, Peptococcus G, AU-2; Streptococcus intermedius, Ruminococcus lactaris, Ruminococcus CO Gemmiger X, Coprococcus BH, —CC; Eubacterium tenue, Eubacterium ramulus, Eubacterium AE, -AG-H, -AG-M, AJ, -BN-1; Bacteroides clostridiiformis ss. clostridliformis, Bacteroides coagulans, Bacteroides orails, Bacteroides ruminicola ss. brevis, -ss. ruminicola, Bacteroides splanchnicus, Desuifomonas pigra, Bacteroides L-4, -N-i; Fusobacterium H, Lactobacillus G, and Succinivibrio A.

Generally, a unicellular cell may be from the domains Bacteria or Archaea. In some cases, a cell may be derived from the domain Eukarya, a multicellular organism, e.g., plants, animals.

In some cases, a medicament can be a small molecule. Classes of small molecule drugs can include an antibiotic agent, an antiviral agent, an antifungal agent, an anti-neoplastic, an anti-inflammatory, a phenethylamine, a 5-alpha reductase inhibitor, a statin, a vitamin, a fibrate, an analgesic, a narcotic, an antidiabetic agent, a diuretic, and any combination thereof.

In some instances, the medicament can be an antibiotic agent. In some exemplary embodiments, an antibiotic agent can be selected from the group consisting of: Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, a salt of any of these, and any combination thereof. In some cases, an additional antiviral agent can be selected from the group consisting of: Acyclovir, Brivudine, Docosanol, Famciclovir, Idoxuridine, Penciclovir, Trifluridine, Valacyclovir, Amantadine, Rimantadine, a neuraminidase inhibitor, Oseltamivir, Zanamivir, a salt of any of these, and any combination thereof.

In some instances, the medicament can be an antiviral agent. In some embodiments, an antiviral agent can be Acyclovir, Brivudine, Cidofovir, Docosanol, Famciclovir, Foscarnet, Fomivirsen, Ganciclovir, Idoxuridine, Penciclovir, Peramivir, Trifluridine, Valacyclovir, Vidarabine, Lamivudine, Ribavirin Amantadine, Rimantadine, a neuraminidase inhibitor, Oseltamivir, Zanamivir, a salt of any of these, or any combination thereof.

In some instances, the medicament can be an anti-inflammatory drug. In some embodiments, the anti-inflammatory can be diclofenac, ketoprofen, ibuprofen, aspirin, a salt of any of these, and any combination thereof.

In some instances, the medicament can be an antifungal agent. In some embodiments, the antifungal agent can include polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine, ciclopirox olamine, haloprogin, tolnaftate, undecylenate, topical nysatin, amorolfine, butenafine, naftifine, terbinafine, or any combination thereof.

In some instances, the medicament can be a narcotic. In some embodiments, the narcotic can be fentanyl, morphine, methadone, etorphine, levophanol, sufentanil, D-Ala², N-MePhe⁴, Gly-ol]-enkephalin (DAMGO), butophanol, buprenorphine, naloxone, naltrexone, D-Phe-Cys-Tyr-D-Trp-Orn-Thr-Pen-Thr-NH (CTOP), diprenorphine, b-funaltrexamine, naloxonazine, nalorphine, pentazocine, nalbuphine, codeine, hydrocodone, oxycodone, nalmephene, a salt of any of these, and any combination thereof.

In some instances, the medicament can be a phenethylamine. In some embodiments, the phenethylamine can be dopamine, epinephrine, norepinephrine, phenylephrine, methylphenidate, amphetamine, a salt of any of these, and any combination thereof.

In some instances, the medicament can be a 5-alpha reductase inhibitor. In some embodiments, the 5-alpha reductase inhibitor can be dutasteride, tamsulosin, finasteride a salt of any of these, and any combination thereof.

In some instances, the medicament can be an antineoplastic. In some embodiments, the antineoplastic can be selected from the group consisting of cyclophosphamide, methotrexate, 5-fluorouracil, doxorubicin, procarbazine, prednisolone, bleomycin, vinblastine, dacarbazine, cisplatin, epirubicin, a salt of any of these, and any combination thereof.

In some instances, the medicament can be a statin. In some embodiments, the statin can be selected from the group consisting of atorvastatin, fluvastatin, lovastatin, pravastatin, simvastatin, rosuvastatin, and any combination thereof.

In some instances, the medicament can be an anti-diabetic agent. In some embodiments, the anti-diabetic agent can be selected from the group consisting of acarbose, miglitol, metformin, alogliptin, canagliflozin, dapagliflozin, empagliflozin, glipizide, glyburide, linagliptin, pioglitazone, repaglinide, rosiglitazone, saxagliptin, sitagliptin, bromocriptine, albiglutide, dulaglutide, exenatide, liraglutide, nateglinide, repaglinide, dapagliflozin, tolazamide, tolbutamide, a salt of any of these, and any combination thereof.

In some instances, the medicament can be a growth factor or a differentiation factor, which induces growth or differentiation of target cells. Examples can include growth hormones, specific tissue growth or differentiation factors, proliferation factors, regeneration factors, and the like. Tissues can include skeletomuscular, e.g., bone, heart, muscle, cartilage, tendon, and the like, skin, eye, neural, brain, tissues of ectoderm, mesoderm, or endoderm origin, as described, e.g., in an anatomy, developmental biology, or histology textbook.

In some instances, a medicament can be a specialty drug. Examples of specialty drugs can include a therapeutic antibody, a protein or peptide therapy, a small molecule, a therapeutic vaccine, a stem cell therapy, or a blood derivative such as an IVIG therapy.

Examples of approved specialty drugs (injectables, oral/topical) to treat diseases including various cancers are: paclitaxel protein-bound; brentuximab vedotin; everolimus; nelarabine, afatumumab; bevacizumab; belinostat; blinatumomab, bosutinib; vandetanib; cabozantinib; progesterone gel; ramucirumab; decitabine; leuprolide acetate; asparaginase; cetuximab; vismodegib; panobinostat; degarelix; pralatrexate; obinutuzumab; alemtuzumab; afatinib; imatinib; eribulin; trastuzumab; topotecan, palbociclib; ponatinib; ibrutinib; axitinib; interferon alpha-2b; romidepsin; ixabepilone; ruxolitinib; cabazitaxel; trastuzumab emtansine; palifermin; pembrolizumab; carfilzomib; levoleucovorin calcium, leuprolide, vincristine sulfate; procarbazine; trametinib; sorafenib; mitoxantrone; pegaspargase; nivolumab; pertuzumab; pomalidomide; aldesleukin; prothelial, sipuleucel-T; mercaptopurine; lenalidomide; rituximab (MabThera); dasatinib; regorafenib; sunitinib; peginterferon alfa-2b; siltuximab; omacetaxine mepesuccinate; dabrafenib; erlotinib; bexarotene; nilotinib; temozolomide; testosterone; thalidomide; thyrotropin alfa; temsirolimus; bendamustine; lapatinib; mechlorethamine; valrubicin; histrelin; panitumumab; bortezomib; azacitidine; pazopanib; crizotinib; capecitabine; denosumab; enzalutamide; ipilimumab, ziv-aflibercept; vemurafenib; goserelin; vorinostat; zoledronic acid; idelalisib; ceritinib; abiraterone; axicabtagene ciloleucel; tisagenlecleucel.

Examples of approved specialty drugs to treat diseases including multiple sclerosis are: dalfampridine; teriflunomide; interferon beta-1a; interferon beta-1b, glatiramer acetate; fingolimod; alemtuzumab; ocrelizumab; mitoxantrone; daclizumab; peginterferon beta-1a; dimethyl fumarate; natalizumab.

Examples of approved specialty drugs to treat diseases including inflammatory indications such as rheumatoid arthritis are: tocilizumab; rilonacept; belimumab; certolizumab pegol; etanercept; vedolizumab, adalimumab; canakinumab; anakinra; pegloticase; abatacept; methotrexate; methotrexate injection; infliximab; rituximab; golimumab; ustekinumab; tofacitinib.

Examples of approved specialty drugs to treat inflammatory bowel diseases such as Crohn's and ulcerative colitis are: certolizumab pegol; vedolizumab; adalimumab; infliximab; golimumab; natalizumab.

Examples of approved specialty drugs to treat diseases including psoriasis are secukinumab, etanercept; adalimumab; apremilast; infliximab; methotrexate injection; ustekinumab.

Examples of approved specialty drugs to treat osteoarthritis can include: hyaluronate sodium; hyaluronate cross-linked; hylan G-F 20.

Examples of approved specialty drugs to treat osteoporosis can include: ibandronic acid, teriparatide; denosumab; zoledronic acid.

An example of an approved specialty drugs to treat systemic lupus erythematosus can include: belimumab.

Examples of approved specialty drugs to treat ophthalmic conditions can include: cysteamine; aflibercept; ocriplasmin; fluocinolone acetonide; ranibizumab; pegaptanib; dexamethasone; verteporfin.

Examples of approved specialty drugs to treat immune deficiency can include: interferon gamma-1b; immune globulin (intravenous or subcutaneous), cytomegalovirus immune globulin; immune globulin infusion (Human) 10% IGIV; or SUBQ injections (trade names include Carimune, Flebogamma, Gamastan S-D, Gamunex-C, Hizentra, Privigen, Octagam).

Examples of approved specialty drugs to treat blood cell deficiency can include: darbepoetin alfa; epoetin alfa; tbo-filgrastim; sargramostim; plerixafor; pegfilgrastim; oprelvekin; romiplastim; epoetin alfa; eltrombopag.

Examples of approved specialty drugs to treat alpha-1 deficiency can include: alpha 1 proteinase inhibitor (trade names include Glassia, Zemaira, Prolastin-C).

Examples of approved specialty drugs in the anticoagulants class can include: fondaparimux, dalteparin; fluindione; apixaban; enoxaparin; rivaroxaban.

An example of a specialty drug to treat heart failure can include: sacubitril/valsartan.

Examples of approved specialty drugs to treat enzyme deficiency and lysosomal storage disorders can include: pegademase bovine; iduronidase, carglumic acid; eliglustat; imiglucerase; cysteamine bitartrate; idursulfase; taliglucerase alfa; agalsidase beta; alglucosidase alfa; galsulfase, nitisinone, sacrosidase; elosulfase alfa, velaglucerase alfa; miglustat.

Examples of approved specialty drugs to treat asthma and allergy can include: omalimumab.

Examples of approved specialty drugs to treat growth deficiency can include: somatropin (trade names include: Humatrope, Saizen, Omnitrope, Zorbtive, Norditropin); mecasermin

Examples of approved specialty drugs to treat hepatitis C virus (HCV) can include: interferon alfacon 1; simeprevir; peginterferon alfa-2a; peginterferon alfa-2b; ribavirin (Rebetol, Copegus, Ribasphere, Ribapak, Moderiba); sofosbuvir; ledipasvir; boceprevir.

Examples of approved specialty drugs to treat human immunodeficiency virus (HIV) can include: tipranavir; efavirenz/emtricitabine/tenofovir; lamivudine/zidovudine, emtricitabine/rilpivirine/tenofovir; indinavir; rilpivirine; tesamorelin; emtricitabine; lamivudine; abacavir/lamivudine; enfuvirtide; etravirine; saquinavir; raltegravir; lopinavir/ritonavir; fosamprenavir, ritonavir; darunavir; delavirdine; zidovudine; atazanavir; maraviroc; elvitegravir/cobicistat/emtricitabine/tenofovir (Quad pill); efavirenz, dolutegravir; abacavir/dolutegravir/lamivudine, abacavir/lamivudine/zidovudine, emtricitabine/tenofovir; cobicistat; nelfinavir; nevirapine; tenofovir disoproxil fumarate; stavudine; abacavir.

Examples of approved specialty drugs to treat pulmonary hypertension can include: tadalafil; riociguat; epoprostenol sodium; ambrisentan; macitentan; treprostinil; sildenafil; bosentan; iloprost.

An example of an approved specialty drug (antibody) to treat respiratory synctial virus can include: palivizumab.

Examples of approved specialty drugs to treat cystic fibrosis can include: tobramycin (inhalation solution); aztreonam; ivacaftor; dornase alfa; lumacaftor/ivacaftor.

Examples of approved specialty drugs to treat infertility can include: urofollitropin, cetrorelix, chorionic Gonadatropin (trade names include Novarel, Pregnyl); progesterone; ganirelix, follitropin, leuprolide, menotropins, choriogonadotropin alfa, progesterone injection.

Examples of approved specialty drugs to treat lipid disorders (PCSK9 inhibitors) can include: alirocumab, evolocumab.

Examples of approved specialty drugs to treat miscellaneous specialty conditions can include: corticotropin injection, apomorphine (movement disorder), minocycline HCl; botulinum toxin, Protein-C concentrate (coagulation disorder), chenodiol; betaine (anhydrous oral solution); teduglutide (gastrointestinal disorders), tasimelteon; lomitapide; mipomersen; sapropterin dihydrochloride (phenylketonuria), hydroxyprogesterone caproate injection (pre-term birth), metreleptin; droxidopa (movement disorder), ziconotide; eculizumab (paroxysmal nocturnal hemoglobinuria); naltrexone; tetrabenazine; incobotulinumtoxinA; collagenase C. histolyticum; sodium oxybate.

Examples of approved specialty drugs to treat hemophilia can include: emicizumab; antihemophilic factor; antihemophilic factor/von Willebrand factor complex [human], Coagulation Factor IX; Factor IX complex; desmopressin; Coagulation Factor XIII A-Subunit (recombinant); antihemophilic and von Willebrand factor complex; antihemophilic factor (recombinant).

Examples of approved specialty drugs to treat endocrine disorders include: testosterone undecanoate; mifepristone; sapropterin; C1 esterase inhibitor; ocretide; pasireotide; lanreotide; pegvisomant; histrelin.

An example of a drug to treat diabetes can include: insulin glargine; sitagliptin.

An example of a drug to treat epilepsy, fibromyalgia, neuropathic pain can include: pregabalin.

In some embodiments, the medicament can include a specialty drug. A specialty drug can be a biologic or small molecule. In some instances, a medicament can be a medicament that at one time was listed in the Orange Book, the Purple Book, or is recited in Table 1, 2, or 3:

Exemplary Specialty Medicaments

paclitaxel protein-bound; brentuximab vedotin; everolimus; nelarabine, ofatumumab; bevacizumab; belinostat; blinatumomab,
bosutinib; vandetanib; cabozantinib; progesterone gel; ramucirumab; decitabine; leuprolide acetate; asparaginase; cetuximab;
vismodegib; panobinostat; degarelix; pralatrexate; obinutuzumab; alemtuzumab; afatinib; imatinib; eribulin; trastuzumab;
topotecan, palbociclib; ponatinib; ibrutinib; axitinib; interferon alpha-2b; romidepsin; ixabepilone; ruxolitinib; cabazitaxel;
trastuzumab emtansine; palifermin; pembrolizumab; carfilzomib; levoleucovorin calcium, leuprolide, vincristine sulfate;
procarbazine; trametinib; sorafenib; mitoxantrone; pegaspargase; nivolumab; pertuzumab; pomalidomide; aldesleukin; prothelial,
sipuleucel-T; mercaptopurine; lenalidomide; rituximab; dasatinib; regorafenib; sunitinib; peginterferon alfa-2b; siltuximab;
omacetaxine mepesuccinate; dabrafenib; erlotinib; bexarotene; nilotinib; temozolomide; testosterone; thalidomide; thyrotropin
alfa; temsirolimus; bendamustine; lapatinib; mechlorethamine; valrubicin; histrelin; panitumumab; bortezomib; azacitidine;
pazopanib; crizotinib; capecitabine; denosumab; enzalutamide; ipilimumab, ziv-aflibercept; vemurafenib; goserelin; vorinostat;
zoledronic acid; idelalisib; ceritinib; abiraterone; axicabtagene ciloleucel; tisagenlecleucel; dalfampridine; teriflunomide;
interferon beta-1a; interferon beta-1b, glatiramer acetate; fmgolimod; alemtuzumab; ocrelizumab; mitoxantrone; daclizumab;
peginterferon beta-1a; dimethyl fumarate; natalizumab; tocilizumab; rilonacept; belimumab; certolizumab pegol; etanercept;
vedolizumab, adalimumab; canakinumab; anakinra; pegloticase; abatacept; methotrexate; methotrexate injection; infliximab;
rituximab; golimumab; ustekinumab; tofacitinib; certolizumab pegol; vedolizumab; adalimumab; infliximab; golimumab; natalizumab;
secukinumab, etanercept; adalimumab; apremilast; infliximab; methotrexate injection; ustekinumab; hyaluronate sodium; hyaluronate
cross- linked; hylan G-F 20; ibandronic acid, teriparatide; denosumab; zoledronic acid; belimumab; cysteamine; aflibercept;
ocriplasmin; fluocinolone acetonide; ranibizumab; pegaptanib; dexamethasone; verteporfin; interferon gamma-1b; immune globulin
(intravenous or subsutaneous), cytomegalovirus immune globulin; immune globulin infusion (Human) 10% IGIV; or SUBQ injections;
darbepoetin alfa; epoetin alfa; tbo-filgrastim; sargramostim; plerixafor; pegfilgrastim; oprelvekin; romiplastim; epoetin
alfa; eltrombopag; an alpha 1 proteinase inhibitor; fondaparimux, dalteparin; fluindione; apixaban; enoxaparin;
rivaroxaban; sacubitril/valsartan; pegademase bovine; iduronidase, carglumic acid; eliglustat; imiglucerase; cysteamine
bitartrate; idursulfase; taliglucerase alfa; agalsidase beta; alglucosidase alfa; galsulfase, nitisinone, sacrosidase;
elosulfase alfa, velaglucerase alfa; miglustat; omalimumab; somatropin; mecasermin; interferon alfacon 1; simeprevir;
peginterferon alfa-2a; peginterferon alfa-2b; ribavirin; sofosbuvir; ledipasvir; boceprevir; tipranavir;
efavirenz/emtricitabine/tenofovir; lamivudine/zidovudine, emtricitabine/rilpivirine/tenofovir; indinavir; rilpivirine;
tesamorelin; emtricitabine; lamivudine; abacavir/lamivudine; enfuvirtide; etravirine; saquinavir; raltegravir;
lopinavir/ritonavir; fosamprenavir, ritonavir; darunavir; delavirdine; zidovudine; atazanavir; maraviroc;
elvitegravir/cobicistat/emtricitabine/tenofovir (Quad pill); efavirenz, dolutegravir; abacavir/dolutegravir/lamivudine,
abacavir/lamivudine/zidovudine, emtricitabine/tenofovir; cobicistat; nelfinavir; nevirapine; tenofovir disoproxil fumarate;
stavudine; abacavir; tadalafil; riociguat; epoprostenol sodium; ambrisentan; macitentan; treprostinil; sildenafil; bosentan;
iloprost; palivizumab; aztreonam; ivacaftor; dornase alfa; lumacaftor/ivacaftor; urofollitropin, cetrorelix, chorionic
Gonadatropin; progesterone; ganirelix, follitropin, leuprolide, menotropins, choriogonadotropin alfa; alirocumab;
evolocumab; corticotropin injection, apomorphine (movement disorder), minocycline HC1; botulinum toxin,
Protein-C concentrate (coagulation disorder), chenodiol; betaine (anhydrous oral solution); teduglutide (gastrointestinal
disorders), tasimelteon; lomitapide; mipomersen; sapropterin dihydrochloride (phenylketonuria), hydroxyprogesterone
caproate injection (pre-term birth), metreleptin; droxidopa (movement disorder), ziconotide; eculizumab (paroxysmal
nocturnal hemoglobinuria); naltrexone; tetrabenazine; incobotulinumtoxinA; collagenase C. histolyticum; sodium oxybate;
emicizumab; antihemophilic factor; antihemophilic factor/von Willebrand factor complex [human], Coagulation
Factor IX; Factor IX complex; desmopressin; Coagulation Factor XIII A-Subunit (recombinant); antihemophilic and
von Willebrand factor complex; antihemophilic factor (recombinant); testosterone undecanoate; mifepristone;
sapropterin; C1 esterase inhibitor; ocretide; pasireotide; lanreotide; pegvisomant; histrelin; sitagliptin; pregabalin.

TABLE 2
Exemplary Therapeutics

Table-2, part A:
The following is a list of the drugs (generic name in parenthesis) coming off patent by:),
2016: Absorica (isotretinoin), Aczone (dapsone), Amitiza (lubiprostone Astagraf XL (tacrolimus), Axiron (testosterone), Azor
(amlodipine/olmesartan), Benicar (olmesartan), Benicar HCT (olmesartan/hct), Canasa (mesalamine suppository), Clindesse
(clindamycin), Crestor (rosuvastatin), Cubicin (daptomycin), Daliresp (roflumilast), Edarbi (azilsartan), Edarbyclor
(azilsartan/chlorthalidone), Effient (prasugrel), Enablex (darifenacin), Epogen (retacrit), Epzicom (abacavir/lamivudine), Erbitux
(cetuximab), Fanapt (iloperidone), Folotyn (pralatrexate), Gleevec (imatinib), Glumetza (metformin er), Humira (adalimumab), Jevtana
(cabazitaxel), Kaletra (lopinavir/ritonavir), Lantus (basalgar), Lantus (solostar basalgar), Letairis (ambrisentan), Lialda (mesalamine dr tab),
Mirvaso (brimonidine), Multaq (dronedarone), Neulasta (pegfilgrastim), Nexavar (sorafenib), Nucynta ER (tapentadol er), Nuvaring (ethinyl
estradiol/etonogestrel), Nuvigil (armodafinil), Oxycontin (oxycodone er), Panretin (alitretinoin), Pennsaid 2% (diclofenac), Potiga (ezogabine),
Proair HFA (albuterol), Procrit (retacrit), Prolensa (bromfenac), Qsymia (phentermine/topiramate), Rayos (prednisone dr), Relpax (eletriptan),
Remicade (infliximab), Savella (milnacipran), Seroquel XR (quetiapine xr), Suboxone Film (buprenorphine/naloxone), Tovzaz (fesoterodine),
Treanda (bendamustine), Tribenzor (amlodipine/olmesartan/hctz), Truvada (emtricitabine/tenofovir), Tygacil (tigecycline), Vascepa
(icosapent ethyl), Viibryd (vilazodone), Ziana (clindamycin/tretinoin), Zytiga (abiraterone).
2017: Acthar Gel (corticotropin), Aggrenox (aspirin/dipyridamole), Alimta (pemetrexed), Alinia (nitazoxanide), Alvesco (ciclesonide),
Ampyra (dalfampridine), Arranon (nelarabine), Aubagio (teriflunomide), Azilect (rasagiline), Butrans (buprenorphine), Byetta (exenatide),
Cancidas (caspofungin), Carbaglu (carglumic acid), Cialis (tadalafil), Combigan (timolol/brimonidine), Contrave (buproprion er/naloxone),
Copaxone 40 mg (glatiramer), Invanz (ertapenem), Liptruzet (atorvastatin/ezetimibe), Macugen (pegaptanib), Naftin (naftifine), Nasonex
(mometasone), Noxafil (posaconazole), Omnaris (ciclesonide), Quillivant XR (methylphenidate), Reyataz (atazanavir), Sabril (vigabatrin),
Sandostatin lar (octreotide), Somavert (pegvisomant), Strattera (atomoxetine), Sustiva (efavirenz), Tamiflu (oseltamivir), Tivicay
(dolutegravir), Uceris tablet (budesonide), Velcade (bortezomib), Viaigra (sildenafil), Victoza (liraglutide), Viread (tenofovir), Vytorin
(ezetimibe/simvastatin), Zetia (ezetimibe), Zioptan (tafluprost), Zolpimist (zolpidem), Zubsolv (buprenorphine/naloxone).
2018: Acanya (benzoyl peroxide/clindamycin), Adcirca (tadalafil), Apidra (insulin glulisine), Astepro (azelastine), Atripla
(efavirenz/emtricitabine/tenofovir), Fentora (fentanyl), Finacea (azelaic acid), Follistim (follitropin beta), Fortesta (testosteronse),
Levitra (vardenafil), Lexiva (fosamprenavir), Lotronex (alosetron), Lyrica (pregabalin), Makena (hydroxyprogesterone), Namzaric
(memantine/donepezil), Pradaxa (dabigatran), Promacta (eltrombopag), Remodulin (treprostinil), Revlimid (lenalidomide), Sensipar tablet
(cinacalcet), Spiriva (tiotripium), Staxyn (vardenafil), Symbicort (budesonide/formoterol), Tekamlo (aliskerin/amlodipine), Tekturna
(aliskerin), Tekturna HCT (aliskerin/hctz), Tikosyn (dofetilide), Treximet (naproxen/sumatriptan), Tyvaso (treprostinil), Vesicare
(solifenacin), Xolair (omalizumab).
2019: Afinitor (everolimus), Avastin (bevacizumab), Azasite (azithromycin), Eliquis (apixaban), Emend INJ (fosaprepitant), Exelon patch
(rivastigmine), Exjade (deferasirox), Factive (gemifloxacin), Firazyr (icatibant), Gilenya (fingolimod), Invega sustenna (paliperidone),
Orencia (abatacept), Prezista (darunavir), Ranexa (ranolazine), Rozerem (ramelteon), Tarceva (erlotinib), Uloric (febuxostat), Xyrem
(sodium oxybate), Zydara (imiquimod).
2020: Atrovent HFA (ipratropium hfa), Byduredon (Exenatide), Chantix (varenicline), Dexilant (dexlansoprazole), Inlyta (axitinib), Namenda
XR (memantine er), Safyral (drospirenone/ethinyl estradiol/levomefolate), Saphris (asenapine), Silenor (doxepin), Sprycel (dasatinib),
Tykerb (lapatinib), Vigamox (moxifloxacin).
2021: Bystolic (nebivolol), Crixivan (indinavir), Emtriva (emtricitabine), Hysingla ER (hydrocodone er), Perforomist (formoterol), Sutent
(sunitinib), Veramyst (fluticasone fuoroate), Xarelto (rivaroxaban), Zomig ns (zolmitriptan).
2022: Januvia (sitagliptin), Oxecta (oxycodone), Pristiq (desvenlafaxine), Selzentry (maraviroc), Victrelis (boceprevir),
Vimovo (esomeprazole/naproxen), Vimpat (lacosamide).
Table-2, part B
Top 5 oncology products worldwide in 2024: Keytruda pembrolizumab; Revlimid lenalidomide; Opdivo nivolumab; Imbruvica
ibrutinib; Ibrance palbociclib.
Top 5 oncology R&D products worldwide in 2024: Tipifarnib tipifarnib; CB-839 (Calithera Biosciences); JCAR017
lisocabtagene maraleucel.
Top 5 anti-rheumatic products worldwide in 2024: Humira adalimumab; Enbrel etanercept; Simponi golimumab; Otezla apremilast;
Upadacitinib upadacitinib tartrate.
Top 5 R&D anti-rheumatic products worldwide in 2024: Upadacitinib upadacitinib tartrate; Filgotinib filgotinib; ONS-3010
adalimumab (Oncobiologics biosimilar); Adalimumab adalimumab (Mylan biosimilar); ABP 710 infliximab (Amgen biosimilar).
Top 50 selling products worldwide in 2024: Humira adalimumab; Keytruda pembrolizumab; Revlimid lenalidomide; Opdivo
nivolumab; Eliquis apixaban; Imbruvica ibrutinib; Ibrance palbociclib; Dupixent dupilumab; Eylea aflibercept; Stelara
ustekinumab; Biktarvy bictegravir sodium, emtricitabine; tenofovir, alafenamide fumarate; Darzalex daratumumab; Tecentriq
atezolizumab; Prolia/Xgeva denosumab; Perjeta pertuzumab; Xarelto rivaroxaban; Ocrevus ocrelizumab; Prevnar 13 pneumococcal
vaccine; Cosentyx secukinumab; Soliris eculizumab; Triumeq abacavir sulfate, dolutegravir sodium, lamivudine; Trulicity
dulaglutide; Botox onabotulinumtoxinA, Xtandi enzalutamide, Hemlibra emicizumab, Entresto sacubitril; valsartan, Ozempic
semaglutide, Enbrel etanercept, Repatha evolocumab, Entyvio vedolizumab, Jakafi ruxolitinib phosphate, Tagrisso osimertinib
mesylate, Imfinzi durvalumab, Jardiance empagliflozin, Tremfya guselkumab, VX-659 + Tezacaftor + Ivacaftor, Genvoya
cobicistat; elvitegravir; emtricitabine; tenofovir alafenamide fumarate; Tresiba insulin degludec; Gardasil human papillomavirus
(HPV) vaccine; Gammagard Liquid immune globulin (human); Aducanumab aducanumab; Tecfidera dimethyl fumarate; Pentacel DTP,
Hib & polio Vaccine; Mavyret glecaprevir; pibrentasvir; Elafibranor elafibranor; Simponi golimumab; Avastin bevacizumab; Venclexta
venetoclax; Tivicay dolutegravir; Taltz ixekizumab.
See also, the FDA Orange Book, Purple Book, or foreign counterpart listings; past, present, and future.

In some cases, a medicament can be a biosimilar to a licensed or approved drug. In some cases, a medicament can be “highly biosimilar” to a licensed or approved drug as defined by the US FDA. In some cases, a medicament can have “no clinically meaningful differences” from a licensed or approved drug as defined by the US FDA. In some cases, a drug can be biosimilar to a specialty drug as described herein. In some cases, a biosimilar can be a drug with homology to an approved or licensed drug. In some cases, a biosimilar can be a drug with homology to a specialty drug as described herein.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=(# of identical positions/total # of positions)×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

In some cases, a medicament can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% homology to a licensed or approved medicament. In some cases, a medicament can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% homology to a specialty drug as described herein. In some cases, a medicament can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% homology to a medicament listed in Table 1, 2, or 3.

In some cases, a biosimilar medicament can have a number of amino acid substitutions relative to a licensed or approved drug. In some cases, a biosimilar medicament can have at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids substitutions relative to a licensed or approved drug.

In some cases, a biosimilar medicament can have a number of amino acid substitutions relative to a specialty drug as described herein. In some cases, a biosimilar medicament can have at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids substitutions relative to a specialty drug as described herein.

In some cases, a biosimilar medicament can have a number of amino acid substitutions relative to a medicament recited in Table 1, 2, or 3. In some cases, a biosimilar medicament can have at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids substitutions relative to a medicament recited in Table 1, 2, or 3.

In some cases, a medicament can comprise a polypeptide sequence of Table 3:

TABLE 3
Exemplary Polypeptide Sequences

LTYTDCTESGQNLCLCEGSNVCGQGNKCILGSDGEKNQCVTGEGTPKP
QSHNDGDFEEIPEEYLQ; FPRPGGGGNGDFEEIPEEYL;
SYQGNSDCYFGNGSAYRGTHSLTESGASCLPWNSMILIGKVYTAQNPS
AQALGLGKHNYC
RNPDGDAKPWCHVLKNRRLTWEYCDVPSCSTCGLRQYSQPQFRIKGG
LFADIASHPWQAAIFAKHRRSPGERFLCGGILISSCWILSAAHCFQERFPP
HHLTVILGRTYRVVPGEEEQKFEVEKYIVHKEFDDDTYDNDIALLQLKS
DSSRCAQESSVVRTVCLPPADLQLPDWTECELSGYGKHEALSPFYSERL
KEAHVRLYPSSRCTSQHLLNRTVTDNMLCAGDTRSGGPQANLHDACQ
GDSGGPLVCLNDGRMTLVGIISWGLGCGQKDVPGVYTKVTNYLDWIR
DNMRP;
SYQVICRDEKTQMIYQQHQSWLRPVLRSNRVEYCWCNSGRAQCHSVP
VKSCSEPRCFNGGTCQQALYFSDFVCQCPEGFAGKCCEIDTRATCYEDQ
GISYRGTWSTAESGAECTNWNSSALAQKPYSGRRPDAIRLGLGNHNYC
RNPDRDSKPWCYVFKAGKYSSEFCSTPACSEGNSDCYFGNGSAYRGTH
SLTESGASCLPWNSMILIGKVYTAQNPSAQALGLGKHNYCRNPDGDAK
PWCHVLKNRRLTWEYCDVPSCSTCGLRQYSQPQFRIKGGLFADIASHP
WQAAIFAKHRRSPGERFLCGGILISSCWILSAAHCFQERFPPHHLTVILG
RTYRVVPGEEEQKFEVEKYIVHKEFDDDTYDNDIALLQLKSDSSRCAQE
SSVVRTVCLPPADLQLPDWTECELSGYGKHEALSPFYSERLKEAHVRL
YPSSRCTSQHLLNRTVTDNMLCAGDTRSGGPQANLHDACQGDSGGPL
VCLNDGRMTLVGIISWGLGCGQKDVPGVYTKVTNYLDWIRDNMRP;
SYQVICRDEKTQMIYQQHQSWLRPVLRSNRVEYCWCNSGRAQCHSVP
VKSCSEPRCFNGGTCQQALYFSDFVCQCPEGFAGKCCEIDTRATCYEDQ
GISYRGNWSTAESGAECTNWQSSALAQKPYSGRRPDAIRLGLGNHNYC
RNPDRDSKPWCYVFKAGKYS SEFCSTPACSEGNSDCYFGNGSAYRGTH
SLTESGASCLPWNSMILIGKVYTAQNPSAQALGLGKHNYCRNPDGDAK
PWCHVLKNRRLTWEYCDVPSCSTCGLRQYSQPQFRIKGGLFADIASHP
WQAAIFAAAAASPGERFLCGGILISSCWILSAAHCFQERFPPHHLTVILG
RTYRVVPGEEEQKFEVEKYIVHKEFDDDTYDNDIALLQLKSDSSRCAQE
SSVVRTVCLPPADLQLPDWTECELSGYGKHEALSPFYSERLKEAHVRL
YPSSRCTSQHLLNRTVTDNMLCAGDTRSGGPQANLHDACQGDSGGPL
VCLNDGRMTLVGIISWGLGCGQKDVPGVYTKVTNYLDWIRDNMRP;
LKIAAFNIQTFGETKMSNATLVSYIVQILSRYDIALVQEVRDSHLTAVGK
LLDNLNQDAPDTYHYVVSEPLGRNSYKERYLFVYRPDQVSAVDSYYY
DDGCEPCGNDTFNREPAIVRFFSRFTEVREFAIVPLHAAPGDAVAEIDAL
YDVYLDVQEKWGLEDVMLMGDFNAGCSYVRPSQWSSIRLWTSPTFQ
WLIPDSADTTATPTHCAYDRIVVAGMLLRGAVVPDSALPFNFQAAYGL
SDQLAQAISDHYPVEVMLK;
ARPCIPKSFGYSSVVCVCNATYCDSFDPPTFPALGTFSRYESTRSGRRME
LSMGPIQANHTGTGLLLTLQPEQKFQKVKGFGGAMTDAAALNILALSP
PAQNLLLKSYFSEEGIGYNIIRVPMASCDFSIRTYTYADTPDDFQLHNFS
LPEEDTKLKIPLIHRALQLAQRPVSLLASPWTSPTWLKTNGAVNGKGSL
KGQPGDIYHQTWARYFVKFLDAYAEHKLQFWAVTAENEPSAGLLSGY
PFQCLGFTPEHQRDFIARDLGPTLANSTHEINVRLLMLDDQRLLLPHWA
KVVLTDPEAAKYVHGIAVHWYLDFLAPAKATLGETHRLFPNTMLFASE
ACVGSKFWEQSVRLGSWDRGMQYSHSIITNLLYHVVGWTDWNLALNP
EGGPNWVRNFVDSPIIVDITKDTFYKQPMFYHLGHFSKFIPEGSQRVGL
VASQKNDLDAVALMHPDGSAVVVVLNRSSKDVPLTIKDPAVGFLETIS
PGYSIHTYLWRRQ;
EFARPCIPKSFGYSSVVCVCNATYCDSFDPPTFPALGTFSRYESTRSGRR
MELSMGPIQANHTGTGLLLTLQPEQKFQKVKGFGGAMTDAAALNILAL
SPPAQNLLLKSYFSEEGIGYNIIRVPMASCDFSIRTYTYADTPDDFQLHN
FSLPEEDTKLKIPLIHRALQLAQRPVSLLASPWTSPTWLKTNGAVNGKG
SLKGQPGDIYHQTWARYFVKFLDAYAEHKLQFWAVTAENEPSAGLLS
GYPFQCLGFTPEHQRDFIARDLGPTLANSTHEINVRLLMLDDQRLLLPH
WAKVVLTDPEAAKYVHGIAVHWYLDFLAPAKATLGETHRLFPNTMLF
ASEACVGSKFWEQSVRLGSWDRGMQYSHSIITNLLYHVVGWTDWNLA
LNPEGGPNWVRNFVDSPIIVDITKDTFYKQPMFYHLGHFSKFIPEGSQRV
GLVASQKNDLDAVALMEIPDGSAVVVVLNRSSKDVPLTIKDPAVGFLET
ISPGYSIHTYLWHRQDLLVDTM;
ADKLPNIVILATGGTIAGSAATGTQTTGYKAGALGVDTLINAVPEVKKL
ANVKGEQFSNMASENMTGDVVLKLSQRVNELLARDDVDGVVITHGTD
TVEESAYFLHLTVKSDKPVVFVAAMRPATAISADGPMNLLEAVRVAGD
KQSRGRGVMVVLNDRIGSARYITKTNASTLDTFKANEEGYLGVIIGNRI
YYQNRIDKLHTTRSVFDVRGLTSLPKVDILYGYQDDPEYLYDAAIQHG
VKGIVYAGMGAGSVSVRGIAGMRKAMEKGVVVIRSTRTGNGIVPPDEE
LPGLVSDSLNPAHARILLMLALTRTSDPKVIQEYFHTY;
ALAQKRDNVLFQAATDEQPAVIKTLEKLVNIETGTGDAEGIAAAGNFL
EAELKNLGFTVTRSKSAGLVVGDNIVGKIKGRGGKNLLLMSHMDTVY
LKGILAKAPFRVEGDKAYGPGIADDKGGNAVILHTLKLLKEYGVRDYG
TITVLFNTDEEKGSFGSRDLIQEEAKLADYVLSFEPTSAGDEKLSLGTSGI
AYVQVNITGKASHAGAAPELGVNALVEASDLVLRTMNIDDKAKNLRF
NWTIAKAGNVSNIIPASATLNADVRYARNEDFDAAMKTLEERAQQKKL
PEADVKVIVTRGRPAFNAGEGGKKLVDKAVAYYKEAGGTLGVEERTG
GGTDAAYAALSGKPVIESLGLPGFGYHSDKAEYVDISAIPRRLYMAARL
IMDLGAGK;
LVPEKEKDPKYWRDQAQETLKYALELQKLNTNVAKNVIMFLGDGMG
VSTVTAARILKGQLHHNPGEETRLEMDKFPFVALSKTYNTNAQVPDSA
GTATAYLCGVKANEGTVGVSAATERSRCNTTQGNEVTSILRWAKDAG
KSVGIVTTTRVNHATPSAAYAHSADRDWYSDNEMPPEALSQGCKDIAY
QLMHNIRDIDVIMGGGRKYMYPKNKTDVEYESDEKARGTRLDGLDLV
DTWKSFKPRYKHSHFIWNRTELLTLDPHNVDYLLGLFEPGDMQYELNR
NNVTDPSLSEMVVVAIQILRKNPKGFFLLVEGGRIDHGHHEGKAKQAL
HEAVEMDRAIGQAGSLTSSEDTLTVVTADHSHVFTFGGYTPRGNSIFGL
APMLSDTDKKPFTAILYGNGPGYKVVGGERENVSMVDYAHNNYQAQS
AVPLRHETHGGEDVAVFSKGPMAHLLHGVHEQNYVPHVMAYAACIG
ANLGHCAPASSLKDKTHTCPPCPAPELLGGPSVFLFPPKPKDTLMISRTP
EVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLP
PSREEMTKNQVSLTCLVKGEYPSDIAVEWESNGQPENNYKTTPPVLDS
DGSFFLYSKLTVDKSRWQQGNVFSCSVMHEALHNHYTQKSLSLSPGKD
IDDDDDDDDDD;
APQPPNILLLLMDDMGWGDLGVYGEPSRETPLCSPSRAALLTGRLPIRN
GFYTTNAHARNLLKKAGYVSKIVGKWHLGHRPQFHPLKHGFNIPVYR
DWEMVGRYYEEFPINLKTGEANLTFLYWAVDATHAPVYASKPFLGTS
QRGRYGDVADNTFVFFTSDNGAALISAPEQGGSNGPFPGHVTAGQVSH
QLGSIMDLFTTSLALAGLTLMDRPIFYYRGDTLMAATLGQHKAHFWT
WTVTTHNLEDHTKLPLIFHLGRDPGERFPLSFEALVPAQPQLNVCNWA
VMNWAPPGCEKLGKPNLDRMAAEGLLFPNFYSANAYTPQEIVGGIPDS
EQLLPEDEWFGSPNCHFGPYDNKARPQIYLQEALDFIKRQARHHPFAVR
EIDDSIGKILELLQDLHLCGKQTTFEGGMREPALAWWPPSDRAIDGLNL
LPTLLQGRNSWENFRQGIDECPGQNVSGASAEYQEALSRITSVVQQHQC
LTPPESIPKKCLWSH;
SGGKLTAVDPETNMNVSEIISYWGFPSEEYLVETEDGYILCLNRIPHGRK
NHSDKGPKPVVFLQHGLLADSSNWVTNLANSSLGEILADAGFDVWMG
NSRGNTWSRKHKTLSVSQDEFWAFSYDEMAKYDLPASINEILNKTGQE
QVYYVGHSQGTTIGFIAFSQIPELAKRIKMFFALGPVASVAFCTSPMAKL
GRLPDHLIKDLFGDKEFLPQSAFLKWLGTHVCTHVILKELCGNLCFLLC
GFNERNLNMSRVDVYTTHSPAGTSVQNMLHWSQAVKFQKFQAFDWG
SSAKNYFHYNQSYPPTYNVKDMLVPTAVWSGGHDWLADVYDVNILLT
QITNLVFHESIPEWEHLDFIWGLDAPWRLYNKIINLMRKYQ;
SMTNETSDRPLVHFTPNKGWMNDPNGLWYDEKDAKWHLYFQYNPND
TVWGTPLFWGHATSDDLTNWEDQPIAIAPKRNDSGAFSGSMVVDYNN
TSGFFNDTIDPRQRCVAIWTYNTPESEEQYISYSLDGGYTFTEYQKNPVL
AANSTQFRDPKVFWYEPSQKWIIVITAAKSQDYKIEIYSSDDLKSWKLES
AFANEGFLGYQYECPGLIEVPTEQDPSKSYWVMFISINPGAPAGGSFNQ
YFVGSFNGTHFEAFDNQSRVVDFGKDYYALQTFFNTDPTYGSALGIAW
ASNWEYSAFVPTNPWRSSMSLVRKFSLNTEYQANPETELINLKAEPILNI
SNAGPWSRFATNTTLTKANSYNVDLSNSTGTLEFELVYAVNTTQTISKS
VFADLSLWFKGLEDPEEYLRMGFEVSASSFFLDRGNSKVKFVKENPYF
TNRMSVNNQPFKSENDLSYYKVYGLLDQNILELYFNDGDVVSTNTYF
MTTGNALGSVNMTTGVDNLFYIDKFQVREVK;
TYKKNDEVEFVRTGYGKDMIKVLHIQRDGKYHSIKEVATTVQLTLSSK
KDYLHGDNSDVIPTDTIKNTVNVLAKFKGIKSIETFAVTICEHFLSSFKH
VIRAQVYVEEVPWKRFEKNGVKHVHAFIYTPTGTHFCEVEQIRNGPPVI
HSGIKDLKVLKTTQSGFEGFIKDQFTTLPEVKDRCFATQVYCKWRYHQ
GRDVDFEATWDTVRSIVLQKFAGPYDKGEYSPSVQKTLYDIQVLTLGQ
VPEIEDMEISLPNIHYLNIDMSKMGLINKEEVLLPLDNPYGKITGTVKRK
LSSRL;
QVQLKQSGPGLVQPSQSLSITCTVSGFSLTNYGVHWVRQSPGKGLEWL
GVIWSGGNTDYNTPFTSRLSINKDNSKSQVFFKMNSLQSNDTAIYYCAR
ALTYYDYEFAYWGQGTLVTVSAASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK;
MGADDVVDSSKSFVMENFSSYHGTKPGYVDSIQKGIQKPKSGTQGNYD
DDWKGFYSTDNKYDAAGYSVDNENPLSGKAGGVVKVTYPGLTKVLA
LKVDNAETIKKELGLSLTEPLMEQVGTEEFIKRFGDGASRVVLSLPFAE
GSSSVEYINNWEQAKALSVELEINFETRGKRGQDAMYEYMAQACAGN
RVRRSVGSSLSCINLDWDVIRDKTKTKIESLKEHGPIKNKMSESPNKTVS
EEKAKQYLEEFHQTALEHPELSELKTVTGTNPVFAGANYAAWAVNVA
QVIDSETADNLEKTTAALSILPGIGSVMGIADGAVHHNTEEIVAQSIALS
SLMVAQAIPLVGELVDIGFAAYNFVESIINLFQVVHNSYNRPAYSPGHK
THAPTSSSTKKTQLQLEHLLLDLQMILNGINNYKNPKLTRMLTFKFYMP
KKATELKHLQCLEEELKPLEEVLNLAQSKNFHLRPRDLISNINVIVLELK
GSETTFMCEYADETATIVEFLNRWITFCQSIISTLT;
MAPTSSSTKKTQLQLEHLLLDLQMILNGINNYKNPKLTRMLTFKFYMP
KKATELKHLQCLEEELKPLEEVLNLAQSKNFHLRPRDLISNINVIVLELK
GSETTFMCEYADETATIVEFLNRWITFCQSIISTLT;
DIQMTQSPSSLSASVGDRVTITCRASQDVNTAVAWYQQKPGKAPKLLI
YSASFLYSGVPSRFSGSRSGTDFTLTISSLQPEDFATYYCQQHYTTPPTFG
QGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEV
THQGLSSPVTKSFNRGEC;
QVQLQQPGAELVKPGASVKMSCKASGYTFTSYNMHWVKQTPGRGLE
WIGAIYPGNGDTSYNQKFKGKATLTADKSSSTAYMQLSSLTSEDSAVY
YCARSTYYGGDWYFNVWGAGTTVTVSAASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKAEPKSCDKTHTCPPCPAPELLG
GPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPGK;
QVQLVESGGGVVQPGRSLRLSCAASGFTFSSYTMHWVRQAPGKGLEW
VTFISYDGNNKYYADSVKGRFTISRDNSKNTLYLQMNSLRAEDTAIYY
CARTGWLGPFDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK;
SDTGRPFVEMYSEIPEIIHMTEGRELVIPCRVTSPNITVTLKKFPLDTLIP
DGKRIIWDSRKGFIISNATYKEIGLLTCEATVNGHLYKTNYLTHRQTNTII
DVVLSPSHGIELSVGEKLVLNCTARTELNVGIDFNWEYPSSKHQHKKLV
NRDLKTQSGSEMKKFLSTLTIDGVTRSDQGLYTCAASSGLMTKKNSTF
VRVHEKDKTHTCPPCPAPELLGGPSVFLFPPKPKDTLMISRTPEVTCVV
VDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYS
KLTVDKSRWQQGNVFSCSVMHEALHNHYTQKSLSLSPG;
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTEDP
WTFGGGTKLEIKGGGGSGGGGSGGGGSQVQLQQSGAELVRPGSSVKIS
CKASGYAFSSYWMNWVKQRPGQGLEWIGQIWPGDGDTNYNGKFKGK
ATLTADESSSTAYMQLSSLASEDSAVYFCARRETTTVGRYYYAMDYW
GQGTTVTVSSGGGGSDIKLQQSGAELARPGASVKMSCKTSGYTFTRYT
MHWVKQRPGQGLEWIGYINPSRGYTNYNQKFKDKATLTTDKSSSTAY
MQLSSLTSEDSAVYYCARYYDDHYCLDYWGQGTTLTVSSVEGGSGGS
GGSGGSGGVDDIQLTQSPAIMSASPGEKVTMTCRASSSVSYMNWYQQ
KSGTSPKRWIYDTSKVASGVPYRFSGSGSGTSYSLTISSMEAEDAATYY
CQQWSSNPLTFGAGTKLELKHHHHHH;
EVQLVESGGKLLKPGGSLKLSCAASGFTFSSFAMSWFRQSPEKRLEWV
AEISSGGSYTYYPDTVTGRFTISRDNAKNTLYLEMSSLRSEDTAMYYCA
RGLWGYYALDYWGQGTSVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK;
QVQLVESGGGVVQPGRSLRLSCAASGFTFSVYGMNWVRQAPGKGLEW
VAIIWYDGDNQYYADSVKGRFTISRDNSKNTLYLQMNGLRAEDTAVY
YCARDLRTGPFDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEAL
HNHYTQKSLSLSPGK

In some cases, a medicament can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% homology to a polypeptide sequence or segment: a protein listed in Table 1, 2, or 3, or a described protein medicament.

In some cases, a medicament can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% length to: a polypeptide sequence or segment: a protein listed in Table 1, 2, or 3, or a described protein medicament.

In some cases, a medicament can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% homology to: a protein listed in Table 1 or 3, or a described protein medicament, and at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% length to: a protein listed in Table 1, 2, or 3, or a described protein medicament.

In some cases, a biosimilar medicament can have a number of amino acid substitutions relative to a medicament recited in Table 3. In some cases, a biosimilar medicament can have at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids substitutions to: a protein listed in Table 1, 2, or 3, or a described protein medicament.

In some cases, a medicament can have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, 99%, or 100% homology to: a protein listed in Table 1, 2, or 3, or a described protein medicament, and can have at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids substitutions to: a protein listed in Table 1, 2, or 3, or a described protein medicament.

In some cases, a medicament can be present in the form of a pharmaceutically acceptable salt. In some instances, a pharmaceutically acceptable salt can be a salt described in Berge et al, J. Pharm. Sci, 1977. In some instances, a pharmaceutically acceptable salts can include those salts prepared by reaction of a peptide with a mineral, organic acid or inorganic base, such salts including, acetate, acrylate, adipate, alginate, aspartate, benzoate, benzenesulfonate, bisulfate, bisulfate, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate and xylenesulfonate.

In some cases, a medicament can be a generic drug as defined by the USFDA. A generic drug can be drug product that can be comparable to a brand/reference listed USFDA-approved drug product in dosage form, strength, route of administration, quality and performance characteristics, and intended use. It may be a medicament in a modified formulation and can be an equivalent. Examples of small molecule drugs can include: lenalidomide, ibrutinib, ruxolitinib, palpociclib, enzalutamide, venetoclax, sofosbuvir, or ledipasvir. In some cases, a medicament can be a biosimilar as defined by the USFDA. A biosimilar can be a biological product that can be highly similar to and has no clinically meaningful differences from an existing USFDA-approved reference product, but may not itself be approved by the USFDA. Examples of biologic drugs can include: rituximab, alemtuzumab, ofatumumab, obinutuzumab, adalimumab, infliximab, etanercept, cetuximab, trastuzumab, pertuzumab, bevacizumab, daratumumab, evolocumab, nivolumab, pembrolizumab, or atezolizumab.

In some cases, a medicament can be a medicament that at one time was recited in a list recited in 42 U.S.C. § 351(1)(3) of the Public Health Services Act or 42 U.S.C § 262(1)(3), which list medicaments provided with some 12-year marketing exclusivity for biologics, and a counterpart list for small molecule medicaments. In some cases, a medicament can be a medicament that at one time was listed in the Orange Book, the Purple Book, or is recited in Table 1, 2, or 3.

Pharmaceutical Compositions

A pharmaceutical formulation disclosed herein can be formulated into a variety of forms and administered by a number of different means. A pharmaceutical formulation can contain conventionally acceptable carriers, adjuvants, and vehicles as desired. The term “parenteral” as used herein can include subcutaneous, intravenous, intramuscular, or intrasternal injection and infusion techniques. Administration can include injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, or intraperitoneal injection.

Solid dosage forms for oral administration can include capsules, tablets, caplets, pills, troches, lozenges, powders, and granules. A capsule can comprise a core material comprising a nutritive protein or composition and a shell wall that encapsulates a core material. In some embodiments a core material can comprise at least one of a solid, a liquid, and an emulsion. In some embodiments a shell wall material can comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer can function as taste-masking agents.

Tablets, pills, and the like can be compressed, multiply compressed, multiply layered, and/or coated. A coating can be single or multiple. In some embodiments, a coating material can comprise at least one of a saccharide, a polysaccharide, and glycoproteins extracted from at least one of a plant, a fungus, and a microbe. Non-limiting examples can include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments a coating material can comprise a protein. In some embodiments, a coating material can comprise at least one of a fat and/or an oil. In some embodiments the at least one of a fat and/or an oil can be high temperature melting. In some embodiments the at least one of a fat and/or an oil can be hydrogenated or partially hydrogenated. In some embodiments the at least one of a fat and/or an oil can be derived from a plant. In some embodiments the at least one of a fat and/or an oil can comprise at least one of glycerides, free fatty acids, and fatty acid esters. In some embodiments a coating material can comprise at least one edible wax. An edible wax can be derived from animals, insects, or plants. Non-limiting examples can include beeswax, lanolin, bayberry wax, carnauba wax, and rice bran wax. Tablets and pills can additionally be prepared with enteric coatings.

Liquid formulations can include a syrup (for example, an oral formulation), an intravenous formulation, an intranasal formulation, an ocular formulation (e.g., for treating an eye infection), an otic formulation (e.g., for treating an ear infection), an ointment, a cream, an aerosol, and the like. In some instances, a combination of various formulations can be administered. In some embodiments, a tablet, pill, and the like can be formulated for an extended release profile.

In some instances, a peptide or salt thereof can be administered in a composition for topical administration. For topical administration, an active agent may be formulated as is known in the art for direct application to a target area. Forms chiefly conditioned for topical application can take the form, for example, of creams, milks, gels, powders, dispersion or microemulsions, lotions thickened to a greater or lesser extent, impregnated pads, ointments or sticks, aerosol formulations (e.g., sprays or foams), hydrogel, soaps, detergents, lotions or cakes of soap. Other conventional forms for this purpose include wound dressings, coated bandages or other polymer coverings, ointments, creams, lotions, pastes, jellies, sprays, and aerosols. Thus, a therapeutic peptide disclosed herein can be delivered via patches or bandages for dermal administration. Alternatively, a peptide can be formulated to be part of an adhesive polymer, such as polyacrylate or acrylate/vinyl acetate copolymer. For long-term applications it might be desirable to use microporous and/or breathable backing laminates, so hydration or maceration of a skin can be minimized. A backing layer can be any appropriate thickness that can provide a desired protective and support functions. A suitable thickness can generally be from about 1 to about 1000 microns. For example, form about 10 to about 300 microns. Topical administration may be in the form of a nail coating or lacquer. For example, an antifungal peptide can be formulated in a solution for topical administration that contains ethyl acetate (NF), isopropyl alcohol (USP), and butyl monoester of poly[methylvinyl ether/maleic acid] in isopropyl alcohol.

Drops, such as eye drops or nose drops, may be formulated with one or more of a therapeutic peptide in an aqueous or non-aqueous base also comprising one or more dispersing agents, solubilizing agents or suspending agents. Liquid sprays can be pumped or conveniently delivered from pressurized packs. Drops can be delivered via a simple eye dropper-capped bottle, via a plastic bottle adapted to deliver liquid contents drop-wise, or via a specially shaped closure.

Ointments and creams may, for example, be formulated with an aqueous or oily base with the addition of suitable thickening and/or gelling agents. Lotions may be formulated with an aqueous or oily base and can in general also contain one or more emulsifying agents, stabilizing agents, dispersing agents, suspending agents, thickening agents, or coloring agents. The percentage by weight of a therapeutic agent in a topical formulation can depend on various factors, but generally can be from 0.01% to 95% of the total weight of the formulation, and typically 0.1-85% by weight.

An aerosol can be employed to administer a peptide or salt thereof to a respiratory tract. For administration by inhalation or insufflation, a composition may take the form of a dry powder, for example, a powder mix of a therapeutic agent and a suitable powder base such as lactose or starch. Therapeutic peptides can also be administered in an aqueous solution when administered in an aerosol or inhaled form. An inhalable formulation can be an inhalable respiratory formulation. Thus, other aerosol pharmaceutical formulations may comprise, for example, a physiologically acceptable buffered saline solution containing between about 0.001 mg/ml and about 100 mg/ml for example between 0.1 and 100 mg/ml, such as 0.5-50 mg/ml, 0.5-20 mg/ml, 0.5-10 mg/ml, 0.5-5 mg/ml or 1-5 mg/ml of one or more of a peptide specific for an indication or disease to be treated.

In some instances, a pharmaceutical formulation can be formulated such that, when a pharmaceutical formulation is administered to a subject, a medicament, salt thereof, or metabolite thereof can be substantially localized in an organ of a subject. An organ can include, but is not limited to: a lung, a bladder, a gall bladder, a heart, a brain, an intestine, a stomach, an ovary, a testicle, a liver, a spleen, or a kidney.

In some embodiments an excipient can be a buffering agent. Non-limiting examples of suitable buffering agents can include sodium citrate, magnesium carbonate, magnesium bicarbonate, calcium carbonate, and calcium bicarbonate. As a buffering agent sodium bicarbonate, potassium bicarbonate, magnesium hydroxide, magnesium lactate, magnesium glucomate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some embodiments an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, IL-3, sodium ortho vanadate, sodium fluoride, N-a-tosyl-Phe-chloromethyl ketone, N-a-tosyl-Lys-chloromethyl ketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol mannitol and water or a combination thereof.

In some embodiments a pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some embodiments a pharmaceutical formulation can comprise a disintegrant as an excipient. In some embodiments a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like.

In some instances, a pharmaceutical formulation can comprise a coloring agent. Non-limiting examples of suitable color agents can include food, drug and cosmetic colors (FD&C), drug and cosmetic colors (D&C), and external drug and cosmetic colors (Ext. D&C). A coloring agent can be used as a dye or a corresponding lake.

In some instances, the pharmaceutical formulation can comprise a chelator. In some cases, a chelator can be a fungicidal chelator. Examples can include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraacetic acid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11] pentatriacontane hexahydrobromide; or triethylenetetramine-N,N,N′,N″,N′″,N′″-hexaacetic acid.

In some instances, a pharmaceutical formulation can comprise a diluent. Non-limiting examples of diluents can include water, glycerol, methanol, ethanol, and other similar biocompatible diluents. In some cases, a diluent can be an aqueous acid such as acetic acid, citric acid, maleic acid, hydrochloric acid, phosphoric acid, nitric acid, sulfuric acid, or similar. In some instances, a diluent can be used to titrate a pH of a peptide to a pH such as physiological pH to produce a salt as described above. In some cases, a diluent can be selected from a group comprising alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, caoline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some embodiments, a pharmaceutical formulation can comprise a surfactant. Surfactants can be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

### Diagnostics and Theragnostics

Pursuant to, for example, 21 U.S.C. § 321(g), the applicable regulations for diagnostic articles can be analogous to those for drugs, particularly “medicaments.” Under the description of “devices,” a component, part, or accessory of the article are similarly treated. In some cases, a diagnostic can be performed in a subject. In some embodiments, a diagnostic can be an assay. An assay can be carried out on a biological sample obtained from a subject. A biological sample may be blood or any excretory liquid. The diagnostic may be to evaluate baseline, to determine standards, to measure control levels, to evaluate rates of change, and other such measures. The diagnostic may cover specific methods, devices, or components within a measuring device. Non-limiting examples of the biological sample may include saliva, blood, serum, cerebrospinal fluid, semen, feces, plasma, urine, a suspension of cells, or a suspension of cells and viruses. A biological sample may contain whole cells, lysed cells, plasma, red blood cells, skin cells, proteins, nucleic acids (e.g., DNA, RNA, maternal DNA, maternal RNA), circulating nucleic acids (e.g., cell-free nucleic acids, cell-free DNA/cfDNA, cell-free RNA/cfRNA), circulating tumor DNA/ctDNA, cell-free fetal DNA/cffDNA). As used herein, the term “cell-free” can refer to the condition of the nucleic acid sequence as it appeared in the body before the sample is obtained from the body. For example, circulating cell-free nucleic acid sequences in a sample may have originated as cell-free nucleic acid sequences circulating in the bloodstream of the human body. In contrast, nucleic acid sequences that are extracted from a solid tissue, such as a biopsy, are generally not considered to be “cell-free.” In some cases, cell-free DNA may comprise fetal DNA, maternal DNA, or a combination thereof. In some cases, cell-free DNA may comprise DNA fragments released into a blood plasma. In some cases, the cell-free DNA may comprise circulating tumor DNA. In some cases, cell-free DNA may comprise circulating DNA indicative of a tissue origin, a disease or a condition. A cell-free nucleic acid sequence may be isolated from a blood sample. A cell-free nucleic acid sequence may be isolated from a plasma sample. A cell-free nucleic acid sequence may comprise a complementary DNA (cDNA). In some cases, one or more cDNAs may form a cDNA library.

In some cases, an assay can include a binding assay. As used herein, a “binding assay” can include a method used to determine an amount of binding of a component of a subject sample with a probe. Methods can include analytic biochemical methods such as electrophoresis, capillary electrophoresis, high performance liquid chromatography (HPLC), thin layer chromatography (TLC), hyperdiffusion chromatography, mass spectroscopy, spectrophotometry, electrophoresis (e.g., gel electrophoresis), and the like. Direct binding can be measured using techniques such as an immunoassay. Examples of immunoassays include immunoprecipitation, particle immunoassays, immunonephelometry, radioimmunoassays, enzyme immunoassays (e.g., ELISA), fluorescent immunoassays, chemiluminescent immunoassays, and Western blot analysis.

In some cases, a diagnostic can be a theragnostic. The term “theragnostic” or “theragnostics” as used herein can refer to products, tests, methods and procedures that can inherently guide treatment in (i) a single subject or (ii) a collection of subjects, e.g., subset(s) of subjects, entire disease-specific population covered by a payer or employer, suffering from a particular disease with a core objective of achieving excellent or near-excellent treatment outcomes in a reasonable timeframe. Such outcomes can include disease remission, cure, excellent response, and the like. Theragnostic procedures can be linked to therapies, treatments, and treatment guidance that collectively dictate efficacy and financial assurances, prior authorization, and the designing of a drug-specific formulary. Such assurances can be offered to payers and employers.

Theragnostic results can be necessary for (a) prior authorization of a specialty drug mandating efficacy and financial assurances; (b) designing and developing a formulary, e.g., disease-specific drug formulary, such that the decision to include or not include a drug in the formulary may be governed by the theragnostic results. A theragnostic product can guide in the selection of drugs, e.g., mechanism of action-based treatment options in specific subset(s) of patients, with an objective of achieving remission or excellent response in defined subset(s) of patients. Some approved drugs with moderate or substandard efficacy profiles may be excluded entirely from the formulary.

A theragnostic methods can be mechanistic: (a) based on the mechanism of action of the drug itself and understanding why a patient or subset(s) of patients respond well given their particular genetic makeup (e.g., the primary therapeutic mechanism of rituximab monotherapy in B-NHL can be ADCC); and (b) based on the pathophysiology of the disease itself as stratified, e.g., according to immunologically defined subtypes of disease (e.g., fibrinogen induced arthritis), disease severity, pharmacology, disease states, and physiology. The resolution of theragnostic procedures can be enhanced by combining more than one mechanistic determinant. A combination of stratification platforms (e.g., the 3×3 matrix based on FcGR-3A VF¹⁵⁸ and FcGR-2A HR¹³¹ polymorphisms), immunological tests, biomarkers, diagnostic tests can be used to generate an indication-specific theragnostic product.

Theragnostics can be employed to (i) select an appropriate therapy for a given patient, given her disease characteristics, when multiple therapies are available to choose from; (ii) decide when not to select a particular therapy for a given patient, given her disease characteristics; (iii) achieve clinical remission or excellent response when the patient is administered with a carefully chosen therapy, e.g., using a particular drug of choice at the first instance. Any or all of the above objectives can be accomplished by the use of theragnostic procedures.

Theragnostic functions can entail: (a) therapeutic appropriateness, which can be the selection of a therapeutic (drug), typically based on use of a particular drug, including a priori, when multiple therapeutics are available in a formulary to choose from, for a particular subset(s) of patients or an individual patient; (b) therapeutic guidance, which provides details of therapy, including aspects of specific drug dosing and schedule details during a treatment cycle; (c) therapeutic effectiveness, which can be a measure of how well the therapy, including the drug, worked in that patient or how well the patient responded to that treatment during and at the end of the treatment cycle; and (d) selection of an alternate therapeutic (drug) that can be considered as the next best choice based on, e.g., a mechanistic rationale, if the first choice failed to achieve reasonable therapeutic effectiveness. Any or all the above objectives can be accomplished by the use of theragnostic procedures.

Furthermore, theragnostic procedures can provide reliable, actionable treatment (and therapeutic) guidance for a single patient (what is generally referred to as precision or individualized medicine), subset(s) and subtype(s) of patients (stratified medicine), as well as for the entire disease population. Theragnostic methods can provide significant advantages to patients (considered one of the payers in the specialty drug context), payers and employers in not only managing diseases and therapies, but also controlling costs both on a per patient basis and for the entire disease population being managed by a payer or employer. Other applications of theragnostics are in the areas of (a) providing therapeutic efficacy and financial assurances to payers, employers and patients; (b) selection of drug formularies as part of the prescription drug plans; (c) product differentiation from other commercially available drugs; and (d) market enrichment for a drug.

In a simpler embodiment, the theragnostic procedures can provide actionable treatment guidance by summary guidelines to achieve preferred outcomes. Thus, the guidance might be summarized by directing specific drug selection (from among alternatives; i.e., therapeutic appropriateness) for defined ranges of theragnostic readouts, directing specific therapy selection (from among alternatives of how drug is administered; i.e., therapeutic guidance) for defined ranges of theragnostic readouts, and directing overall therapy strategy (from among alternatives; i.e., therapeutic effectiveness) for defined ranges of theragnostic readouts, and specific exclusion criteria (from among alternatives; i.e., selection of alternative therapeutic) for particular other theragnostic readouts where treatment strategy may be contraindicated (e.g., by toxicity or side effect) or first strategy fails. Thus, the guidelines may implicitly incorporate the theragnostic-guidance criteria with specific actionable directives based on theragnostic evaluations.

A single or a combination of DNA, RNA, protein, or immunological features may constitute a theragnostic product or evaluation. In addition, it may include metabolic evaluation, which may be useful for individualized pharmacology of half-life, absorption, distribution, metabolism, excretion, turnover, and the like. Such examples can include biomarkers, polymorphisms, gene expression profiles, protein expression profiles, presence or absence of specific protein markers or immunological, metabolic, physiological profiles, and many aspects which affect the therapy response. Furthermore, a single or a combination of companion diagnostic tests or in vitro diagnostic tests (e.g., theragnostics, complementary diagnostics) may constitute a theragnostic procedure.

Tests can include biomarker tests (syn: complementary or companion diagnostic tests, theragnostic tests, and the like, as defined elsewhere by others) have distinct insufficiencies in providing therapeutic and/or economic value. For example, specialty drugs in the checkpoint inhibitors class such as pembrolizumab, nivolumab, and atezolizumab block the interaction between the receptor programmed cell death protein 1 (PD1) on CD8+ T cells and its ligand (PDL1) on tumor cells. Whereas cancer cells co-opt this immune checkpoint pathway to limit T cell activity, the drugs can remove this ‘brake’ and unleash the immune system on the cancer. Although responses to these therapies can be dramatic and durable in melanoma, only about one-third of patients respond. Response rates are significantly lower in non-small-cell lung cancer (NSCLC) and kidney cancer, at approximately 20-25% (Cancer Cell 27, 450-461; 2015).

Drug developers are consequently keen to identify biomarkers that can boost outcomes. While tumor PDL1 expression was an obvious first biomarker candidate, it has not lived up to expectations. PDL1 levels, as measured by immunohistochemistry (IHC), can identify groups of patients that are more likely to respond to PD1-PDL1 blockade, but it is not itself an absolute (e.g., reliable) marker: some patients with high PDL1 levels do not respond to treatment, and contrarily, a subset of those who test negative for PDL1 expression can derive considerable treatment benefit. This underscores the ambiguity around the use of biomarker(s) as to the insufficiency of providing therapeutic or economic value.

Such ambiguities may also be observed in other therapies, e.g., cetuximab (K-RAS mutations in metastatic colorectal cancer versus NSCLC), trastuzumab (Her-2 expression with a 3+ score in breast cancer).

Given this, regulatory approvals are restricted to the use of such a biomarker test for a therapy in a specific indication. PDL1 IHC can be approved as a companion diagnostic only for pembrolizumab in NSCLC. Underscoring the ambiguity around the biomarker, FDA has approved it as a ‘complementary diagnostic’ in melanoma and for nivolumab in NSCLC, to assist but not dictate treatment decision-making. In part, the value of the biomarker may be limited by technical pitfalls such as irregular expression levels throughout the tumor and lack of a single, standardized IHC test. But a more fundamental limitation can be that tumor expression of PDL1 does not provide the whole picture (Nature Rev. Cancer 16, 275-287; 2016).

The major insufficiencies of currently available biomarker or diagnostic tests can be: (a) they do not provide efficacy assurance (e.g., assured remission or excellent response); (b) they do not provide financial assurance; (c) not used for providing assurance-based payment (outcome) decisions; (d) not used for designing and developing a drug formulary by a prescription drug plan; (e) not used for providing disease-specific, population-wide therapy decisions (e.g., involving multiple therapies in a large patient population). Theragnostic procedures delineated herein can address these insufficiencies.

Cell- and Tissue-Based Articles; Gene Therapy; Prophylactic; Devices

Besides classical “medicaments”, the Federal Government carefully regulates the manufacture, use, offer to sell, sale, and/or import of other articles. Among these articles can included cell- and tissue-based compositions and methods, e.g., cell infusions or depletions. Various cell infusions may include mostly unprocessed materials, such as whole blood, to highly purified subpopulations which may be selected (e.g., cell sorting, or the like) or developed (in vivo or in vitro or a combination, including with added differentiation and/or maturation factors, and the like.) for infusion or replacement. Particular methods may be used to generate, e.g., activated cell types, stem cells, differentiated cell populations or purified cell types, cells of defined or particular properties or functions, selected or purified cell products, endogenous or exogenous populations, and the like. Depletion may be useful in, e.g., oncology indications, where cell proliferation is excessive of various types which can be relatively easily removed. In some situations, organs or parts thereof may be transplanted or grafted into a target patient. Such may induce a desired effect in the patient, or may provide a needed organ function, which may replace or supplement a preexisting organ. Common organ transplants include bone marrow, blood transfusions, heart, lung, kidney, spleen, skin, limbs, fingers, toes, intestines, eye, blood vessel, pancreas, and other anatomical or functional organs. In some instance, an organ transplant can include crude nerve replacement, e.g., in degenerative or other conditions.

Gene therapy articles, compositions, and methods can be useful in certain situations. Thus, supplementing genetic issues, or replacing or substituting genetic functions may be desired. The therapy may be direct application of the nucleic acids for uptake by the target subject's own cells, or may include infusion of cells transformed with the nucleic acids so the subject's cells might not actually take up the foreign nucleic acids, but the transformed cells release a desired product into the host organism. In certain cases, the cells become factories releasing, e.g., hormones or factors into the host organism to provide a desired result.

Among prophylactic articles, may include vaccines which prevent or mitigate infection or effects of a particular environmental hazard. The article may be a contraceptive device, e.g., and IUD or slow release contraceptive article. Killing methodologies may be useful, e.g., using radioactive pellets or the like, which can kill surrounding tissue, e.g., cancerous tissue, so placement of the pellets may be important.

Among devices may include implants such as bone or muscle replacement. They may be for repair of joint function, and may be cartilage, tendon, or the like. The implant may have other functions, e.g., heart pacemakers, insulin release, drug release devices, and the like. Joint replacement has become more common and artificial hips, shoulders, knees, and the like are not rare. Other devices may be jigs for the fitting or adjustment of other implants, e.g., support structures to immobilize joints from spinal or other joint surgeries. Other implants may replace or supplement sensory functions, which may include hearing, feeling, sight, smell, taste, or the like. Other implants, e.g., dental implants, provide replacements for teeth. Certain devices may be useful for temporary use, longer term, or permanent use, as the situation warrants. Substitute or replacement components of a device or implant may also be subject to regulation, e.g., power sources, parts which might wear or deteriorate over time, or parts or software components which might be improved or upgraded over the life of the device, or replacement of some consumables (e.g., slow release or controlled release administration).

Prostheses include artificial limbs, or hands or feet. Spinal cord injuries often cause paralysis, and prostheses exist to supplement or replace lost function. Other prostheses are designed to restore function from brain injuries or lost brain function. In the future, brain-mechanical control systems will be developed and become more common for many different applications.

Diseases and Conditions

A condition can be a disease. A disease can be stroke or stroke associated disease. A disease can be ischemic stroke. In some cases, a disease can be Alzheimer's disease or Parkinson's disease. In some cases, a disease can be an autoimmune disease such as acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune idiopathic thrombocytopenic purpura (ITP), autoimmune thyroid disease, axonal & neuronal neuropathies, Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal osteomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophilic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome, subacute bacterial endocarditis (SBE), sympathetic ophthalmia (sympathetic uveitis), Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some cases, a disease can be a cancer such as Acute lymphoblastic leukemia, Acute myeloid leukemia, adrenocortical carcinoma, AIDS-related cancers, AIDS-related lymphoma, anal cancer, appendix cancer, astrocytoma, childhood cerebellar or cerebral, basal cell carcinoma, bile duct cancer, extrahepatic, bladder cancer, bone cancer, osteosarcoma/malignant fibrous histiocytoma, brainstem glioma, brain tumor, brain tumor, cerebellar astrocytoma, brain tumor, cerebral astrocytoma/malignant glioma, brain tumor, ependymoma, brain tumor, medulloblastoma, brain tumor, supratentorial primitive neuroectodermal tumors, brain tumor, visual pathway and hypothalamic glioma, breast cancer, bronchial adenomas/carcinoids, burkitt lymphoma, carcinoid tumor, childhood, carcinoid tumor, gastrointestinal, carcinoma of unknown primary, central nervous system lymphoma, primary, cerebellar astrocytoma, childhood, cerebral astrocytoma/malignant glioma, childhood, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor, childhood, extragonadal germ cell tumor, extrahepatic bile duct cancer, eye cancer, intraocular melanoma, eye cancer, retinoblastoma, gall bladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, glioma, childhood visual pathway and hypothalamic, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood, intraocular melanoma, islet cell carcinoma (endocrine pancreas), Kaposi sarcoma, kidney cancer (renal cell cancer), laryngeal cancer, leukemias, leukemia, acute lymphoblastic (also called acute lymphocytic leukemia), leukemia, acute myeloid (also called acute myelogenous leukemia), leukemia, chronic lymphocytic (also called chronic lymphocytic leukemia), leukemia, chronic myelogenous (also called chronic myeloid leukemia), leukemia, hairy cell, lip and oral cavity cancer, liver cancer (primary), lung cancer, non-small cell, lung cancer, small cell, lymphomas, lymphoma, AIDS-related, lymphoma, Burkitt, lymphoma, cutaneous T-cell, lymphoma, Hodgkin, lymphomas, non-Hodgkin (an old classification of all lymphomas except Hodgkin's), lymphoma, primary central nervous system, Marcus whittle, Deadly disease, Waldenström macroglobulinemia, malignant fibrous histiocytoma of bone/osteosarcoma, medulloblastoma, childhood, melanoma, melanoma, intraocular (Eye), Merkel cell carcinoma, mesothelioma, adult malignant, mesothelioma, childhood, metastatic squamous neck cancer with occult primary, mouth cancer, multiple endocrine neoplasia syndrome, childhood, multiple myeloma/plasma cell neoplasm, mycosis fungoides, myelodysplastic syndromes, myelodysplastic/myeloproliferative diseases, myelogenous leukemia, chronic, myeloid leukemia, adult acute, myeloid leukemia, childhood acute, myeloma, multiple (cancer of the bone-marrow), myeloproliferative disorders, chronic, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer, islet cell, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood, pituitary adenoma, plasma cell neoplasia/Multiple myeloma, pleuropulmonary blastoma, primary central nervous system lymphoma, prostate cancer, rectal cancer, renal cell carcinoma (kidney cancer), renal pelvis and ureter, transitional cell cancer, retinoblastoma, rhabdomyosarcoma, childhood, salivary gland cancer, sarcoma, Ewing family of tumors, sarcoma, Kaposi, sarcoma, soft tissue, sarcoma, uterine, skin cancer (nonmelanoma), skin cancer (melanoma), skin carcinoma, Merkel cell, small cell lung cancer, small intestine cancer, soft tissue sarcoma, squamous cell carcinoma—see skin cancer (nonmelanoma), squamous neck cancer with occult primary, metastatic, stomach cancer, supratentorial primitive neuroectodermal tumor, childhood, T-cell lymphoma, cutaneous—see mycosis fungoides and Sézary syndrome, testicular cancer, throat cancer, thymoma, childhood, thymoma and thymic carcinoma, thyroid cancer, thyroid cancer, childhood, transitional cell cancer of the renal pelvis and ureter, trophoblastic tumor, gestational, unknown primary site, carcinoma of, adult, unknown primary site, cancer of, childhood, ureter and renal pelvis, transitional cell cancer, urethral cancer, uterine cancer, endometrial, uterine sarcoma, vaginal cancer, visual pathway and hypothalamic glioma, childhood, vulvar cancer, Waldenström macroglobulinemia, and Wilms tumor (kidney cancer).

In some cases, a disease can be inflammatory disease, infectious disease, cardiovascular disease and metabolic disease. Specific infectious diseases include, but is not limited to AIDS, anthrax, botulism, brucellosis, chancroid, chlamydial infection, cholera, coccidioidomycosis, cryptosporidiosis, cyclosporiasis, diphtheria, ehrlichiosis, arboviral encephalitis, enterohemorrhagic Escherichia coli, giardiasis, gonorrhea, dengue fever, haemophilus influenza, Hansen's disease (Leprosy), hantavirus pulmonary syndrome, hemolytic uremic syndrome, hepatitis A, hepatitis B, hepatitis C, human immunodeficiency virus, legionellosis, listeriosis, lyme disease, malaria, measles. meningococcal disease, mumps, pertussis (whooping cough), plague, paralytic poliomyelitis, psittacosis, Q fever, rabies, rocky mountain spotted fever, rubella, congenital rubella syndrome (SARS), shigellosis, smallpox, streptococcal disease (invasive group A), streptococcal toxic shock syndrome, Streptococcus pneumonia, syphilis, tetanus, toxic shock syndrome, trichinosis, tuberculosis, tularemia, typhoid fever, vancomycin intermediate-resistant Staphylococcus aureus, varicella, yellow fever, variant Creutzfeldt-Jakob disease (vCJD), Ebola hemorrhagic fever, Echinococcosis, Hendra virus infection, human monkeypox, influenza A, H5N1, lassa fever, Marburg hemorrhagic fever, Nipah virus, O'nyong fever, Rift valley fever, Venezuelan equine encephalitis and West Nile virus.

Additional conditions can include wounds, injuries, accidents, and aging or other related deterioration for which any diagnostics, companion diagnostics, theragnostics, cell- or tissue-based materials or therapies, gene therapies, replacement devices, implants, prostheses, implants, and such may be useful. This can include blood and other cell transfusions, cell infusions, organ transplants, skin transplants (artificial or natural skin sources), muscle or skeletal repair materials (including bone, tendon, cartilage, joint, and the like). Sensory replacement implants may be used, which may include hearing, sight, touch augmentation or replacement.

Methods of Performing a Clinical Trial

Disclosed herein are methods of performing a clinical trial. In some embodiments, a method of conducting a clinical trial can comprise enrolling at a fixed number of subjects in a clinical trial. In some embodiments, at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 subjects are enrolled in a clinical trial. In some embodiments, at least about 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 subjects are enrolled in a clinical trial. In some embodiments, at least about 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, or 100000 subjects are employed in a clinical trial. In some embodiments, at least about 120000, 150000, 180000, 200000, 230000, 260000, 300000, 330000, 360000, 390000, 400000, 420000, 450000, 500000, 600000, 700000, 710000, 720000, 730000, 740000, 750000, 760000, 770000, 780000, 790000, 800000, 810000, 820000, 830000, 840000, 850000, 860000, 870000, 880000, 890000, 900000, 910000, 920000, 930000, 940000, 950000, 960000, 970000, 980000, 990000, or 1000000 subjects are employed in a clinical trial. In some embodiments, at least about 15,000 subjects are enrolled in a clinical trial. Sizeable, large scale trials, e.g., measured by numbers of participants, or significantly long duration trials, e.g., measured by years, are of interest, including those which are directed to comparing, e.g., biosimilar or small molecule generics as alternative to a reference biologic or reference small molecule, including a reference drug from Table 1, 2, or 3. These numbers of subjects may also be limitations for upper limit sizes of “limited” subsets or arms of studies, e.g., for prior studies in pay-to-participate type trials. Or these same numbers may serve as lower limit sizes of “sizeable” subsets or arms of studies, e.g., of distinguishing pay-to-participate trials.

Reference is made to the following applications: “Novel Healthcare Delivery, Treatment, and Payment Model for Specialty Drugs” (Healthcare) PCT/US16/63681, filed on Nov. 23, 2016, U.S. Provisional Application No. 62/412,102, filed Oct. 24, 2016; U.S. Provisional Application No. 62/365,317, filed on Jul. 21, 2016; and U.S. Provisional Application No. 62/259,291, filed on Nov. 24, 2015.

In some embodiments, a clinical trial can be conducted in the United States, wholly or in part. In some embodiments, a clinical trial may not be conducted fully in the United States, including parts in the EU (Europe, including UK, Germany, France, Spain, Italy, and/or Sweden), Canada, Mexico, Japan, China, South Korea, or India. In some embodiments, a clinical trial may be conducted, wholly or in part, in any one (or combination) of Afghanistan, Albania, Algeria, Andorra, Angola, Antigua and Barbuda, Argentina, Armenia, Australia, Austria, Azerbaijan, Bahamas, Bahrain, Bangladesh, Barbados, Belarus, Belgium, Belize, Benin, Bhutan, Bolivia, Bosnia and Herzegovina, Botswana, Brazil, Brunei, Bulgaria, Burkina Faso, Burundi, Cabo Verde, Cambodia, Cameroon, Canada, Central African Republic, Chad, Chile, China, Colombia, Comoros, Democratic Republic of the Congo, Republic of the Congo, Costa Rica, Cote d'Ivoire, Croatia, Cuba, Cyprus, Czech Republic, Denmark. Djibouti, Dominica, Dominican Republic, Ecuador, Egypt, El Salvador, Equatorial Guinea, Eritrea, Estonia, Ethiopia, Fiji, Finland, France, Gabon, Gambia, Georgia, Germany, Ghana, Greece, Grenada, Guatemala, Guinea, Guinea-Bissau, Guyana, Haiti, Honduras, Hungary, Iceland, India, Indonesia, Iran, Iraq, Ireland, Israel, Italy, Jamaica, Japan, Jordan, Kazakhstan, Kenya, Kiribati, Kosovo, Kuwait, Kyrgyzstan, Laos, Latvia, Lebanon, Lesotho, Liberia, Libya, Liechtenstein, Lithuania, Luxembourg, Macedonia (FYROM), Madagascar, Malawi, Malaysia, Maldives, Mali, Malta, Marshall Islands, Mauritania, Mauritius, Mexico, Micronesia, Moldova, Monaco, Mongolia, Montenegro, Morocco, Mozambique, Myanmar (Burma), Namibia, Nauru, Nepal, Netherlands, New Zealand, Nicaragua, Niger, Nigeria, North Korea, Norway, Oman, Pakistan, Palau, Palestine, Panama, Papua New Guinea, Paraguay, Peru, Philippines, Poland, Portugal, Qatar, Romania, Russia, Rwanda, Saint Kitts and Nevis, Saint Lucia, Saint Vincent and the Grenadines, Samoa, San Marino, Sao Tome and Principe, Saudi Arabia, Senegal, Serbia, Seychelles, Sierra Leone, Singapore, Slovakia, Slovenia, Solomon Islands, Somalia, South Africa, South Korea, South Sudan, Spain, Sri Lanka, Sudan, Suriname, Swaziland, Sweden, Switzerland, Syria, Taiwan, Tajikistan, Tanzania, Thailand, Timor-Leste, Togo, Tonga, Trinidad and Tobago, Tunisia, Turkey, Turkmenistan, Tuvalu, Uganda, Ukraine, United Arab Emirates, United Kingdom, United States, Uruguay, Uzbekistan, Vanuatu, Vatican City, Venezuela, Vietnam, Yemen, Zambia, or Zimbabwe. In some embodiments, a clinical trial may not be conducted in any one of Afghanistan, Albania, Algeria, Andorra, Angola, Antigua and Barbuda, Argentina, Armenia, Australia, Austria, Azerbaijan, Bahamas, Bahrain, Bangladesh, Barbados, Belarus, Belgium, Belize, Benin, Bhutan, Bolivia, Bosnia and Herzegovina, Botswana, Brazil, Brunei, Bulgaria, Burkina Faso, Burundi, Cabo Verde, Cambodia, Cameroon, Canada, Central African Republic, Chad, Chile, China, Colombia, Comoros, Democratic Republic of the Congo, Republic of the Congo, Costa Rica, Cote d'Ivoire, Croatia, Cuba, Cyprus, Czech Republic, Denmark. Djibouti, Dominica, Dominican Republic, Ecuador, Egypt, El Salvador, Equatorial Guinea, Eritrea, Estonia, Ethiopia, Fiji, Finland, France, Gabon, Gambia, Georgia, Germany, Ghana, Greece, Grenada, Guatemala, Guinea, Guinea-Bissau, Guyana, Haiti, Honduras, Hungary, Iceland, India, Indonesia, Iran, Iraq, Ireland, Israel, Italy, Jamaica, Japan, Jordan, Kazakhstan, Kenya, Kiribati, Kosovo, Kuwait, Kyrgyzstan, Laos, Latvia, Lebanon, Lesotho, Liberia, Libya, Liechtenstein, Lithuania, Luxembourg, Macedonia (FYROM), Madagascar, Malawi, Malaysia, Maldives, Mali, Malta, Marshall Islands, Mauritania, Mauritius, Mexico, Micronesia, Moldova, Monaco, Mongolia, Montenegro, Morocco, Mozambique, Myanmar (Burma), Namibia, Nauru, Nepal, Netherlands, New Zealand, Nicaragua, Niger, Nigeria, North Korea, Norway, Oman, Pakistan, Palau, Palestine, Panama, Papua New Guinea, Paraguay, Peru, Philippines, Poland, Portugal, Qatar, Romania, Russia, Rwanda, Saint Kitts and Nevis, Saint Lucia, Saint Vincent and the Grenadines, Samoa, San Marino, Sao Tome and Principe, Saudi Arabia. Senegal, Serbia, Seychelles, Sierra Leone, Singapore, Slovakia, Slovenia, Solomon Islands, Somalia, South Africa, South Korea, South Sudan, Spain, Sri Lanka, Sudan, Suriname, Swaziland, Sweden, Switzerland, Syria, Taiwan, Tajikistan, Tanzania, Thailand, Timor-Leste, Togo, Tonga, Trinidad and Tobago, Tunisia, Turkey, Turkmenistan, Tuvalu, Uganda, Ukraine, United Arab Emirates, United Kingdom, United States, Uruguay, Uzbekistan, Vanuatu, Vatican City, Venezuela, Vietnam, Yemen, Zambia, or Zimbabwe.

In some cases, a subject can be enrolled in a clinical trial for a defined period. In some cases, a subject can be enrolled in the clinical trial for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some cases, a subject can be enrolled in the clinical trial for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In some cases, as subject can be enrolled in a clinical trial for at least about 1, 2, 3, 4, 5, 6, 7, 8, 9, 10, 11, or 12 months. In some cases, a subject can be enrolled in a clinical trial for at least about 1, 2, 3, 4, 5, 6, 7, 8, 9, or 10 years. In some embodiments, a subject can be enrolled in a clinical trial for a combination of years, months, and days. In some embodiments, a clinical trial, e.g., pay-to-participate trial, can be performed for at least about 7 years. In some embodiments, a clinical trial may not be performed for at least about 7 years.

A clinical trial as described herein can be conducted by a provider, e.g., a third-party clinical trial sponsor. In some cases, the provider of the clinical trial may be a drug manufacturer, a hospital, a clinic, or a healthcare provider. In some cases, the third-party sponsor of the clinical trial may not be a drug manufacturer, a hospital, a clinic, or a healthcare provider. In some cases, the clinical trial sponsor is a third-party sponsor, e.g., a managed care company. In some embodiments, the third-party sponsor is responsible for designing, managing, and/or executing the clinical trial; the trial data developed by the third-party sponsor are owned by the sponsor. In some embodiments, a sponsor is the sole representative to communicate with a regulatory authority, e.g., FDA, for the clinical trial matters. In some embodiments, the financial and/or legal liabilities related to the clinical trial rest with the third-party sponsor. In some cases, a provider can be an independent payer. A payer, e.g., third-party payer, can be an entity with financial ties to a subject to be enrolled in a clinical trial. In some cases, a third-party payer can be an insurer, a government insurance agency, a government healthcare entity, an employer, a pension fund, and the like. In some cases, a payer can be a non-profit managed care consortium, e.g., Kaiser Permanente. An insurer can include public or private insurance plans. A government insurance agency or government healthcare entity can include Medicare, Medicaid, the Veteran's Administration, a Home Health Agency, and the like. Examples of a government healthcare insurance agency in Europe can include the National Health Service (England), the Statutory Health Insurance (Gesetzliche Krankenversicherung; Germany), and the like. Counterpart entities exist in Canada, Sweden, Mexico, Japan, China, South Korea, India, and elsewhere. In some cases, an employer may be a provider through an employment group health plan or a paycheck deduction from the subject. In some cases, a subject may be a third-party payer in combination with one or more other payers. In some cases, a subject can provide a copayment or coinsurance payment for a clinical trial.

In some embodiments, a third-party clinical trial sponsor can be paid on a subject-by-subject basis or collective bundle only if or when the medicament shows efficacy. In some embodiments, a provider can be paid at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 US dollars per dose of a medicament. In some embodiments, a provider can be paid at least about 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 US Dollars per dose of a medicament. In some embodiments, a provider can be paid at least about 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 200000, 400000, 600000, 700000, 800000, 900000, 1000000, 200000, or 3000000 US Dollars per dose of a medicament. In some cases, a provider may not be paid if the medicament shows efficacy. In some cases, a provider can be paid less than a full expected amount if efficacy is less. In some cases, a provider may only be paid for so long as a medicament shows efficacy in a subject.

In some embodiments, a clinical trial participation cost can be paid by a third-party payer essentially on a subject-by-subject basis based on efficacy, e.g., therapeutic efficacy such as remission, excellent response, and the like. In some embodiments, a clinical trial participation cost can be paid by a payer on a subject-by-subject basis not based on efficacy. In some embodiments, a clinical trial participation cost can be paid by a payer not on a subject-by-subject basis, e.g., semi-bundled basis within an acceptable small percentage close to individual subject-by-subject accounting. In some embodiments, a third-party payer can pay on a pay-as-you-go basis or in multiple installments. In some embodiments, a payer may not pay on a pay-as-you-go or in multiple installments, e.g., all due at beginning of treatment. In some embodiments, a payer can pay a participation cost of at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 US dollars. In some embodiments, a payer can pay a participation cost of at least about 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 US Dollars. In some embodiments, a third-party payer can pay a participation cost of at least about 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 200000, 400000, 600000, 700000, 800000, 900000, 1000000, 200000, or 3000000 US Dollars. In some embodiments, a payer can be a party who does not at least partially, or substantially, own or have licensed intellectual property to a medicament administered in a clinical trial, its formulation, or its method of use. In some embodiments, a payer can be a party who does at least partially own or have licensed intellectual property to a medicament administered in a clinical trial, its formulation, or its method of use. In some embodiments, a third-party payer may not be an entity that paid a contract manufacturer of a medicament. In some embodiments, a third-party payer can be an entity that paid a contract manufacturer of a medicament.

In some embodiments, a clinical trial can be a prospective clinical trial. In some embodiments, a clinical trial may not be a prospective clinical trial. In some embodiments, a clinical trial can be a retrospective clinical trial. In some embodiments, a clinical trial may not be a retrospective clinical trial. In some embodiments, a clinical trial can be a diagnostic only clinical trial, or may compare multiple diagnostic modalities. In some embodiments, a clinical trial may not be a diagnostic only clinical trial.

In some embodiments, a clinical trial can be conducted under the guidance of a manufacturer of the medicament. In some embodiments, a clinical trial may not be conducted under the guidance of a pharmaceutical company commercializing the medicament. In some embodiments, a clinical trial can be conducted by a clinical trial provider. In some embodiments, a clinical trial can be conducted by a third-party clinical trial sponsor. In some embodiments, a clinical trial sponsor and the clinical trial provider are the same. In some embodiments, a clinical trial can be funded or sponsored or conducted by a government research entity such as National Institute of Health, National Cancer Institute, and the like. In some embodiments, a trial may be funded, wholly or in part, by a third-party payer e.g., an insurer, government payer, pension fund, who may pay all or part of clinical trial costs, or either therapeutic (drug) cost and/or other participation costs, e.g., administrative, non-drug therapy, associated materials, or related costs.

In some embodiments, at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 subjects had participated in a prior art Phase I-IV clinical trial, having been supported by one of the drug company sponsor, a healthcare provider, on a pay-to-participate basis, or a payer. In some embodiments, participation by any one of those categories can make up more than about 1, 2, 3, 4, 5, 6, 7, 8, 9, 10, 11, 12, 13, 14, 15, 16, 17, 18, 19, 20, 21, 22, 23, 24, 25, 27, 29, 31, 33, 35, 37, 39, 41, 45, 48, 51, 55, 60, 65, 70% or more of the participants in the trial or relevant arm of such trial.

In some embodiments, a regulatory agency has authorized a clinical trial provider to charge for a medicament. In some embodiments, a regulatory agency has authorized a third-party clinical trial sponsor to charge for a medicament. In some instances, a regulatory agency can be, e.g., the US Food and Drug Administration (FDA), the European Medicines Agency (EMEA), the Chinese State Food and Drug Administration, the Indian Central Drug Standard Control Organization (CDSCO), or the Japanese Ministry of Health, Labour & Welfare (MHLW).

In some embodiments, a subject may remain in a clinical trial for the entire duration of the clinical trial. In some cases, a subject may be allowed to remain in a clinical trial only if the medicament shows efficacy in the subject. In some cases, a subject not showing efficacy can be administered an alternative medicament, followed by additional determinations of efficacy. In some cases, a trial may be ended, and the subject transferred to a different trial. In some cases, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 subjects do not show efficacy. In some embodiments, at least about 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 subjects do not show efficacy. In some embodiments, at least about 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, or 100000 subjects do not show efficacy. In some cases, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 subjects show efficacy. In some embodiments, at least about 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 subjects show efficacy. In some embodiments, at least about 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, or 100000 subjects show efficacy. In some cases, at about 0.1%, 0.2%, 0.3%, 0.4%, 0.5%, 0.6%, 0.7%, 0.8%, or 0.9% of subjects do not show efficacy. In some cases, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of subjects do not show efficacy. In some cases, at about 0.1%, 0.2%, 0.3%, 0.4%, 0.5%, 0.6%, 0.7%, 0.8%, or 0.9% of subjects show efficacy. In some cases, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of subjects show efficacy.

In some embodiments, a method described herein can include performing at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 clinical trials in parallel or simultaneously. In some embodiments, a method described herein can include performing at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 clinical trial arms in parallel or simultaneously. In some cases, clinical trial arms can be joined into a single clinical trial. In some cases, clinical trial arms may not be joined into a single clinical trial. In other cases, a meta study may combine initially distinct trials into a dataset which can be evaluated as a single larger trial for certain purposes or questions.

A clinical trial can include administration of one or more medicaments as described herein. In some embodiments, only 1 medicament may be given in a clinical trial. In some embodiments, more than 1 medicament can be given in a clinical trial. In some cases, a medicament can be administered via an administration paradigm. In some embodiments, an administration paradigm can comprise administration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 medicaments. In some embodiments, an administration paradigm can comprise more than 1 medicament given sequentially. In some embodiments, an administration paradigm may not comprise more than 1 medicament given sequentially. In some embodiments, an administration paradigm can comprise more than 1 medicament given simultaneously as a combination. In some embodiments, an administration paradigm may not comprise more than 1 medicament given simultaneously as a combination. In some embodiments, an administration paradigm can comprise one or more medicaments given via the same route of administration. In some embodiments, an administration paradigm may not comprise one or more medicaments given via the same route of administration. In some embodiments, an administration paradigm can comprise one or more medicaments given via the different routes of administration. In some embodiments, an administration paradigm may not comprise one or more medicaments given via the different routes of administration. In some embodiments, a medicament administered in a clinical trial may have the same administration paradigm as another medicament given in the clinical trial. In some embodiments, a medicament administered in a clinical trial may not have the same administration paradigm as another medicament given in the clinical trial. As used herein, a “route of administration” can include injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration.

In some embodiments, data obtained from a clinical trial can be input into a database comprised in a system described herein. In some embodiments, a database can comprise clinical trial data obtained from the clinical trial. In some embodiments, a database may not comprise clinical trial data obtained from the clinical trial. In some embodiments, a database can comprise diagnostic data obtained from the clinical trial. In some embodiments, a database may not comprise diagnostic data obtained from the clinical trial, or data other than clinical trial data. In some embodiments, a database can comprise diagnostic data not obtained from the clinical trial. In some embodiments, a database may not comprise diagnostic data not obtained from the clinical trial. In some embodiments, a database may comprise theragnostic data. In some embodiments, a database may not comprise theragnostic data.

A clinical trial can include controls typical of trials. Such controls can include subject randomization, use of placebos, use of double blinds, and the like. In some cases, a clinical trial may not have any one or all of controls typical of trials. In some cases, a clinical trial can be a randomized clinical trial (RCT). In some cases, a clinical trial can be an adaptive clinical trial (ACT). In some cases, a clinical trial can be a prospective, double-blinded clinical trial. In some cases, a clinical trial can be a retrospective, double-blinded clinical trial. In some cases, a trial may not be a subject randomized, placebo controlled, double blind study. In some instances, it can be a retrospective study of a disease registry.

In trials, whether 271(e) or non-271(e) clinical trials, patient samples may be collected and stored for further retrospective analyses that might be carried out in the future. In one embodiment, such patient samples include DNA, RNA, serum, plasma, or other tissue samples, e.g., frozen at −20 or −80 degree Celsius. In another embodiment, such retrospective analyses may be blinded or non-blinded statistical analyses. In one instance, through blinded retrospective trial analyses, a biomarker that may be discovered in the future, e.g., a DNA or RNA biomarker, B-cell biomarker, a subset B-cell biomarker, T-cell biomarker, or an immune biomarker, e.g., a citrullinated antibody, or cytokine expression levels in inflammatory macrophages, can be used to correlate treatment response or disease severity, e.g., non-small cell lung cancer or multiple sclerosis. In another embodiment, clinical data obtained from such retrospective trials can be submitted to regulatory agencies, e.g., FDA, for label revisions. In yet another embodiment, an objective of such retrospective trials is to correlate long-term, e.g., 7-15 year longitudinal outcomes on treatment outcomes and or disease severity to a theragnostic product or a biomarker.

While much of this section describes “clinical trials”, the scope of the disclosure can encompass activities which are for “uses reasonably related to the development and submission of information under a Federal law which regulates the manufacture, use, or sale of drugs or veterinary biological products”. These activities may be distinct from a classical clinical trial, in that they may have different goals and endpoints. In particular, there may be other contexts in which the Federal law may desire information and/or submission of information about medical methods, medicaments, devices, and the like. In some instances, Federal law can regulate the manufacture, use, offer to sell, sale of articles which may not be classically considered “drugs” as the term is commonly understood by the lay public. For instance, 21 U.S.C. § 321 (g) and (h) describe drugs and devices, which can include articles intended for use in the diagnosis, cure, mitigation, treatment, or prevention of disease; and an instrument, apparatus, implement, machine, contrivance, implant, in vitro reagent, or other similar article, including any component, part, or accessory. Thus, besides “medicaments”, the methods described herein may be applicable to, e.g., diagnostic articles and methods, companion diagnostics, theragnostics, cell- and tissue-based articles and methods, selected cell populations, stem cell populations, organs, cell- and tissue-based infusions, transfusions, transplants, grafts, attachments, implants, vaccine and other preventative articles and methods, gene therapy articles and methods, medical devices and components thereof, implants, jigs, fitting devices, prostheses, instrumentation which is attached to patients in treatment, prevention, monitoring, diagnosis, cure, and the like. These can include methods and equipment used to manufacture, use, offer to sell, sell any of the articles or subarticles.

In some cases, a successful clinical trial can be followed up by marketing efforts for a medicament. Marketing efforts for a medicament may include pricing, promotion and/or product placement. For example, a business may encourage physicians to preferentially prescribe patient specific therapies. Such marketing efforts to health care providers, including physicians, nurses, hospitals and medical insurance providers may include, for example, continuing medical education about the therapy, advertisements about the therapy placed in peer-review journals, print/internet advertising or direct sale calls. Additionally or alternatively, marketing efforts may be directed to patients, including patients with a disease or disorder may include, for example, print, television, internet and/or radio advertisements.

FIG. 2 provides an exemplary overview of a method described here. A patient population can be referred for treatment in a clinical trial by a third-party payer, such as an insurer, healthcare provider, or an employer. Costs for treatment in a clinical trial can be borne by a third-party payer, such as an insurer, an employer, a pension fund, or similar entities. A subset of the patient population that are available for treatment are stratified using a platform. Drugs that are approved and licensed by a regulatory agency, such as the US FDA, are obtained, e.g., purchased at fair market value from pharmaceutical companies. In order to stratify subjects, diagnostic methods can be used. In some cases, biomarkers or other diagnostic products or tests can be imported into the platform from diagnostic companies in order to stratify the patient population in the platform. After entering the platform, the patient population can be stratified into those that are to receive treatment using conventional FDA approved or licensed drugs, and those that will enter an Extended Clinical (EC) platform to enter a clinical trial.

FIG. 3A and FIG. 3B depict exemplary components of the platform. The platform can employ, for example, a drug formulary, a disease and therapy management care team, and a theragnostic lab to provide prior authorization for the subject to enter the EC platform or receive a conventional treatment. In one embodiment, most of the clinical trial functions including disease and therapy management (DTM), patient therapeutic adherence (PTA) and therapy guideline adherence (TGA) by specialist physicians involved in clinical trials are managed through the virtual (telehealth) clinical platform. In some cases, a PTA can average from about 80% to about 90%, which would encompass about 75%, 85%, or 95%.

A drug formulary can contain an electronic database of available treatments, as well as a physical storage or pharmacy that stocks the treatments. A drug formulary can have drugs that are approved and licensed by a regulatory agency, as well as drugs that are not licensed or not approved by the regulatory agency. A drug that may be non-licensed or non-approved by a regulatory agency can become licensed or approved by the regulatory agency through a clinical trial.

A theragnostic lab can be a facility capable of performing a diagnostic method to determine a specific disease state in the patient population. The theragnostic lab can employ markers (e.g., biomarkers) or assays obtained from diagnostic companies, as well as diagnostic tests that can be developed in house. Diagnostic tests can be those that have been approved and or licensed by a regulatory agency, for example, as a companion diagnostic. A diagnostic test can also be non-licensed or non-approved diagnostics that can become approved by a regulatory agency after conducting a clinical trial. Various trials may be performed to compare, optimize, or perfect among different diagnostic strategies.

A disease and therapy management team can be a group of healthcare professionals, such as a specialty doctor or a specialty nurse, which can interpret results obtained from the theragnostic lab. The disease and clinical management team can access the drug formulary provide a prior authorization based at least in part on the data obtained from the theragnostic lab as well as the treatments available in the drug formulary. For example, a prior authorization can include allowing a subject to receive a conventional treatment, allowing a subject to enter a clinical trial to receive a non-licensed or non-approved drug, or allowing the subject to choose between the two options.

A subject receiving a licensed or approved drug may be covered by a traditional insurance payer system, or may pay out of pocket for the drug. However, a subject entering a clinical trial may not follow a traditional insurance payer system. In some cases, a third-party payer responsible for costs of treatment may not be charged for a non-licensed or non-approved drug administered in a clinical trial if the non-licensed or non-approved drug fails to show efficacy in the subject. Payers therefore may only be charged on a subject-by-subject basis based on the efficacy of the drug.

After entering the EC (or clinical trial) platform, a subject can be enrolled in a clinical trial. FIG. 4 depicts a patient population enrolled in the clinical trial can be further stratified based on a disease subtype. Non-licensed or non-approved drugs that are biosimilar to approved and licensed drugs can be administered to subjects based on projected efficacy against disease subtypes. Patients who do not show remission after a fixed amount of time can be administered additional drugs biosimilar to existing approved and licensed drugs. This treatment paradigm can continue until a subject achieves remission or fails to achieve remission.

Data obtained from the clinical trial can be input into a clinical trial system. The clinical trial data can be used to support an application to a regulatory agency for approval of the drug based on the clinical trial data subject to exemptions to patent protection such as provisions outlined in 35 U.S.C. § 271(e) in the United States. Patent protection for drugs developed in clinical trials can be pursued based on the clinical trial data and novel formulations that can be developed therein.

Evergreening Strategies:

Innovators, e.g., primary patent holders or brand-name pharmaceutical companies adopt various strategies to extend their patent portfolio(s) to cover products (e.g., to replace patents that may be about to expire), in order to retain market monopoly over those products, by either obtaining new, additional patents called secondary and tertiary patents. This can be referred to herein as an evergreening strategy.

This strategy may be used by brand-name pharmaceutical companies of an innovator drug, e.g., a blockbuster drug, to restrict or prevent competition from manufacturers of generic equivalents or biosimilars. Such secondary and tertiary patents can provide serial barriers to the entry of generics and biosimilars. One such example of a blockbuster biologic drug is adalimumab, shielded by 60-100 secondary and or tertiary patents.

Primary patents directly protect the active ingredient of the drug to be administered to treat a disease indication by claiming rights to the chemical sequence, amino acid sequence, and composition of matter. For instance, a human monoclonal antibody, e.g., an IgG₁ isotype, that binds to CD20 antigen in human B-cells can be protected as a primary patent. Such a primary patent may cover specific amino acid and or nucleotide sequences of the complementarity determining regions (CDR₁₋₃) of the variable heavy (V_{H}) and light (V_{L}) chains of the antibody. Besides covering antibody, such a patent may also cover the antigen, an epitope, or paratope. For small molecule drugs, such a primary patent may cover the chemical structure, chirality, mass spectrometric data, solution structure as determined by 1D or 2D NMR spectroscopy, or the crystal structure as determined by x-ray crystallography of a small molecule drug itself or a co-complex structure of a drug and the macromolecule to which it binds. Such a sequence or structure may be referred to as the composition of matter in the case of biologic drugs and the active ingredient in the case of small molecule drugs. In addition to the active ingredient, primary patents may also cover pharmacological, preclinical, or clinical features of a drug in a disease indication. The end of patent exclusivity, for instance, the twenty-year term limit of the primary patent, can be referred to as patent cliff. Owing to generic competition, the drug price may fall steeply by as much as 60-90% after patent cliff.

Secondary patents can protect a drug peripherally or in some instances, marginally or incrementally, by claiming rights to methods of use, formulations, dosages, methods of manufacturing, and the like. Such secondary patents can include, for instance, improvements or variations over associated drug delivery systems, e.g., intravenous versus subcutaneous delivery, or new pharmaceutical variants, e.g., isomers, salts, conformers, tautomers, analogs, derivatives, isotopes, anomers, chemical or structural polymorphs, solvates, metabolites, intermediates, prodrugs, chemical conversion structures, minor variant chemical or biological structures; amino acid or glycosylation sequence variations; drug metabolites upon administration in humans e.g., venlafaxine and desvenlafaxine; combination drugs (e.g., comprising two patent protected drugs, or one of two drugs is not covered by any patents); or variations in buffers, formulations, excipients, pH, and the like. Additional strategies may include modifying manufacturing methods or modifying how the drug is used, administered, or target subjects. In one instance, evergreening may involve one or multiple secondary patents, e.g., 10-50 or more secondary patents. Such examples may include modified manufacturing processes, e.g., a glycoengineered eukaryotic cell line for manufacturing an antibody with slightly different glycosylation properties, which may or may not lead to improved clinical and (or) therapeutic benefits. In some instances, such therapeutic benefits may be incremental, e.g., about 1-2%, 5%, or 10% enhanced ADCC and may not translate to significantly improved clinical or therapeutic outcomes.

In another instance, as defined herein, evergreening may also involve one or multiple tertiary patents protecting combination products, e.g., drug-device combination product, drug-companion diagnostics combination product. Many of the marketed drug-device products are covered by only tertiary device patents and not by primary or secondary patents. Another form of combination product may include an enzyme as part of the coformulation strategy, e.g., human hyaluronidase, which aids better dispersion and absorption of complex biologics such as antibodies or immunoglobulins when administered subcutaneously.

Tertiary patents can be distinguishable from other types of patents and can increasingly become a core strategy of intellectual property protection on drugs delivered through inhalers and injector pens once primary and secondary patents expire. In addition, because there may be no restriction to the number of times a device can be altered, and such an alteration is a patentably-distinct but not clinically important feature, the potential for sequential device modification and repeated patenting can be feasible. Thus, in one embodiment, secondary patents may outlast and conceivably outnumber primary patents as they are typically filed at a later point in time. In another embodiment, tertiary patents may also outnumber and outlast secondary patents, and in some cases by many years.

In some instances, the terms secondary and tertiary patents may be used interchangeably. In one instance, a glycoengineered eukaryotic CHO cell line used for manufacturing a therapeutic antibody may be characterized as a secondary or tertiary patent.

Approximately 74% of new drug patents in FDA records between 2005 and 2015 were awarded to existing drugs, not for creating new medicines. Of the top 100 pharma products by sales, rather than creating new medicines, pharmaceutical companies are recycling and repurposing old ones. On average, 78% of the drugs associated with new patents in the FDA's records were not new drugs entering the market, but existing drugs. Second, adding new patents and exclusivities to extend the patent cliff to delay the date of loss of patent protection, e.g., due to expiration, is particularly pronounced among blockbuster drugs. Of the roughly 100 best-selling drugs, where the financial incentives of extending are greatest, almost 80% extended their protection at least once, with almost 50% extending the term of patent cliff more than once. Third, once a company begins to exploit this strategy, there is a tendency to repeat this process, where the return on investment is substantial.

In another example of evergreening strategy, a biomarker or a companion diagnostic test can be added to the drug label, e.g., an innovator's blockbuster drug. Such kits or products are protected by additional patents, e.g., tertiary patents, and may subset patients according to treatment response or toxicity response, and the like.

Since branded drugs are gaining lengthy and lucrative evergreening extensions, small molecule generics and biosimilars, e.g., albeit approved by FDA, find it difficult to reach or access the market; hence, the innovator drugs continue to maintain market monopoly for an extended additional period, e.g., 2-5 years, and in some instances, 10-20 years.

Such an evergreening strategy can also be employed to protect devices, e.g., delivery devices such as inhalers or injector pens, diagnostic kits, implants, cell therapeutics, blood-derived therapeutic products such as IVIG therapies, and the like. In one instance, such secondary patents in the device space may cover second generation nanomaterials, biopolymer coatings, anti-microbial coatings, and the like.

Accordingly, in one embodiment, the inventive steps of the clinical trials, e.g., both 271(e) and non-271(e) clinical trial settings described herein can carry the exemption provision from secondary patent infringement. In one embodiment, the inventive steps of the clinical trials described herein can carry the exemption provision from tertiary patent infringement. In another embodiment, the inventive steps of the clinical trials described herein can carry the exemption provision from both secondary and tertiary patent infringement. In yet another embodiment, the inventive steps of the clinical trials described herein can carry the exemption provision from primary patent infringement. In another embodiment, the inventive steps of the clinical trials, described herein can carry the exemption provision from primary, secondary, and tertiary patent infringement. In an embodiment, the inventive steps of the clinical trials described herein can carry the exemption provision for a drug that is not covered by market exclusivity.

Exemplary Activities Providing Exemption from Patent Infringement; Clinical Trials

In some embodiments, “uses reasonably related to the development and submission of information under a Federal law which regulates the manufacture, use, or sale of drugs or veterinary biological products” can be a clinical trial. Thus, in some embodiments a method can comprise conducting a clinical trial, e.g., which can comprise at least about 16,000 subjects enrolled in a clinical trial, where the clinical trial can be in the U.S., where the clinical trial can be conducted for at least 7 years, and where a medicament can be administered in the clinical trial that may not be a prophylactic vaccine. In some embodiments, a method of conducting a clinical trial may not comprise at least about 16,000 subjects enrolled in a clinical trial, where the clinical trial can be in the US, where the clinical trial can be conducted for at least 7 years, and where a medicament can be administered in the clinical trial that may not be a prophylactic vaccine. In some embodiments, a medicament being tested in the trial can be a medicament, or a biosimilar to a reference biologic, recited in Table 1, 2, or 3.

In some embodiments, a method of conducting a clinical trial can comprise at least about 15,000 subjects enrolled in a clinical trial, where the clinical trial can be in the US, where the clinical trial can be conducted for at least 7 years, where a medicament can be administered to a subject, and where the medicament can be a specialty drug; wherein the specialty drug treats a complex, chronic, rare, or difficult to manage disease or disorder. In some embodiments, a method of conducting a clinical trial may not comprise at least about 15,000 subjects enrolled in a clinical trial, where the clinical trial can be in the US, where the clinical trial can be conducted for at least 7 years, where a medicament can be administered to a subject, and where the medicament can be a specialty drug; wherein the specialty drug treats a complex, chronic, rare, or difficult to manage disease or disorder.

In some embodiments, a method of conducting a clinical trial can comprise administering a medicament to a subject within the US, where a clinical trial participation cost can be paid by a third-party payer on a subject-by-subject basis based on efficacy, where the payer pays on a pay-as-you-go basis or in multiple installments, where at least one subject does not show efficacy, and where the third-party payer can be a party who does not at least partially own or have licensed intellectual property to the medicament, its formulation, or its method of use. In some embodiments, a method of conducting a clinical trial may not comprise administering a medicament to a subject within the US, where a clinical trial participation cost can be paid by a third-party payer on a subject-by-subject basis based on efficacy, where the third-party payer pays on a pay-as-you-go basis or in multiple installments, where at least one subject does not show efficacy, and where the third-party payer can be an entity who does not at least partially own or have licensed intellectual property to the medicament, its formulation, or its method of use.

In some embodiments, a method of conducting a clinical trial can comprise a clinical trial where: (a) a medicament can be administered to a subject, (b) the clinical trial provider may only be paid if the medicament shows efficacy in the subject, for so long as the medicament shows efficacy in the subject, on a subject-by-subject basis, wherein at least one subject does not show efficacy, and (c) the third-party payer can be a party who does not at least partially own or have licensed intellectual property to the medicament, its formulation, or its method of use.

In some embodiments, a method of conducting a clinical trial can comprise a clinical trial where: (a) a medicament can be administered to a subject, (b) the clinical trial provider may only be paid if the medicament shows efficacy in the subject, for so long as the medicament shows efficacy in the subject, on a subject-by-subject basis, where at least one subject does not show efficacy, and (c) the third-party payer can be a party who does not at least partially own or have licensed intellectual property to the medicament, its formulation, or its method of use.

In some embodiments, a method of performing a clinical trial can comprise: conducting at least 4 clinical trials in parallel, where: (a) each of the at least 4 clinical trials employs a medicament, (b) the medicament in each of the at least 4 clinical trials can be different, (c) each medicament can be targeted to the same disease or condition, (d) at least one medicament can be a non-licensed and non-approved medicament, (e) one third-party sponsor sponsors all of the at least 4 clinical trials, and (i) a subject stays in one of the at least 4 clinical trials for as long as the medicament in that clinical trial shows efficacy in the subject, or (ii) the subject can be placed into a different one of the at least 4 clinical trials when the medicament shows decreased or no efficacy. In some embodiments, a method of performing a clinical trial may not comprise: conducting at least 4 clinical trials in parallel, where: (a) each of the at least 4 clinical trials employs a medicament, (b) the medicament in each of the at least 4 clinical trials can be different, (c) each medicament can be targeted to the same disease or condition, (d) at least one medicament can be a non-licensed and non-approved medicament, (e) one third-party sponsor sponsors all of the at least 4 clinical trials, and (i) a subject stays in one of the at least 4 clinical trials for as long as the medicament in that clinical trial shows efficacy in the subject, or (ii) the subject can be placed into a different one of the at least 4 clinical trials when the medicament shows decreased or no efficacy.

In some embodiments, a method of performing a clinical trial can comprise, conducting at least 4 clinical trials simultaneously, where: (a) each of the at least 4 clinical trials are directed towards the same disease or condition, (b) the medicament in each of the at least 4 clinical trials can be different, (c) the at least 4 clinical trials run for at least about 10 years, and (d) the at least 4 clinical trials have the same third-party sponsor. In some embodiments, a method of performing a clinical trial may not comprise, conducting at least 4 clinical trials simultaneously, where: (a) each of the at least 4 clinical trials are directed towards the same disease or condition, (b) the medicament in each of the at least 4 clinical trials can be different, (c) the at least 4 clinical trials run for at least about 10 years, and (d) the at least 4 clinical trials have the same sponsor.

In some embodiments, a method of performing a clinical trial can comprise, conducting a clinical trial with at least 3 clinical trial arms run in parallel, where: (a) each of the at least 3 clinical trial arms employs a medicament, (b) the medicament in each of the at least 3 clinical trial arms are the same, (c) the medicament can be a non-licensed and non-approved medicament, (d) the medicament in each of the at least 3 clinical trial arms are administered via a different administration paradigm; and (i) a subject stays in one of the at least 3 clinical trial arms when the medicament shows efficacy in the subject, or (ii) the subject can be placed into a different one of the at least 3 clinical trial arms when the medicament shows decreased or no efficacy. In some embodiments, a method of performing a clinical trial may not comprise, conducting a clinical trial with at least 3 clinical trial arms run in parallel, where: (a) each of the at least 3 clinical trial arms employs a medicament, (b) the medicament in each of the at least 3 clinical trial arms are the same, (c) the medicament can be a non-licensed and non-approved medicament, (d) the medicament in each of the at least 3 clinical trial arms are administered via a different administration paradigm; and (i) a subject stays in one of the at least 3 clinical trial arms when the medicament shows efficacy in the subject, or (ii) the subject can be placed into a different one of the at least 3 clinical trial arms when the medicament shows decreased or no efficacy.

In some embodiments, a method of performing a clinical trial can comprise, conducting a clinical trial, where (a) data from the clinical trial can be periodically entered into a clinical trial database, (b) a subject can be administered a medicament and stays on the medicament in the clinical trial as long as the medicament shows efficacy, and (c) the clinical trial provider can be paid for efficacy, on a subject-by-subject basis by a payer, where the third-party payer may not be the subject or can be a party who does not at least partially own or have licensed intellectual property to the medicament, its formulation, or its method of use. In some embodiments, a method of performing a clinical trial may not comprise, conducting a clinical trial, where (a) data from the clinical trial can be periodically entered into a clinical trial database, (b) a subject can be administered a medicament and stays on the medicament in the clinical trial as long as the medicament shows efficacy, and (c) the clinical trial provider can be paid for efficacy, on a subject-by-subject basis by a third-party payer, where the third-party payer may not be the subject or can be a party who does not at least partially own or have licensed intellectual property to the medicament, its formulation, or its method of use.

In some embodiments, a method can comprise administering a non-approved and non-licensed medicament to a subject in a therapeutically effective amount in a clinical trial, where the medicament clinical trial provider can be reimbursed for the medicament based on efficacy on a subject-by-subject basis, and wherein the reimbursement may not be provided by the manufacturer of the medicament or the subject. In some embodiments, a method may not comprise administering a non-approved and non-licensed medicament to a subject in a therapeutically effective amount in a clinical trial, where the medicament clinical trial provider can be reimbursed for the medicament based on efficacy on a subject-by-subject basis, and wherein the reimbursement may not be provided by the manufacturer of the medicament or the subject.

In some embodiments, a method can comprise administering a non-approved and non-licensed medicament by performing a clinical trial as described herein, where the non-licensed and non-approved medicament was taken to trial but discontinued; and generating data or licensing for the non-licensed and non-approved medicament based at least in part from a clinical trial. In some embodiments, a method may not comprise administering a non-approved and non-licensed medicament by performing a clinical trial as described herein, where the non-licensed and non-approved medicament was taken to trial but discontinued; and generating data or licensing for the non-licensed and non-approved medicament based at least in part from a clinical trial.

In some embodiments, a method can comprise administering a medicament to a subject in a clinical trial, where the medicament was approved or licensed for a first condition, and where the administering can be effective to at least partially treat a second condition that may not be the first condition, where the medicament clinical trial provider can be reimbursed for the medicament based on efficacy on a subject-by-subject basis. In some embodiments, a method may not comprise administering a medicament to a subject in a clinical trial, where the medicament was approved or licensed for a first condition, and where the administering can be effective to at least partially treat a second condition that may not be the first condition, where the medicament clinical trial provider can be reimbursed for the medicament based on efficacy on a subject-by-subject basis.

Federal Law(s) Regulating Manufacturing, Use, and/or Sale of Drugs

In many instances, a significant majority of the FDA-approved, commercially marketed drugs, e.g., expensive specialty drugs, achieve significant therapeutic response in less than 5-20% of the treated patients in all-comers setting. In other instances, FDA is approving novel targeted therapies, e.g., specialty drugs, at an accelerated pace and employs expedited pathways such as the fast-track process and breakthrough therapy designation. In some cases, the expedited pathway can be an accelerated approval, an accelerated access, a priority review, a fast track, a breakthrough therapy program, or the like. The agency now requires fewer and smaller clinical trials, approving some drugs after just one successful Phase 3 clinical trial. The agency also accepts short-term endpoints, e.g., whether a drug shrinks a tumor, instead of long-term clinical outcomes, e.g., whether the drug prolongs life as measured by progression-free survival, and ever-smaller improvements in health as sufficient proof that an oncology drug works and is worth selling. The thousand-plus cancer drugs now in clinical development (see www.clinicaltrials.gov) are often quite likely to help only a handful of patients. A great majority of the targeted cancer therapies will benefit <5% of de novo cancer patients or in disease-relapsed settings as determined by next-generation sequencing, e.g., 1-2 percent of the cancer patients they are aimed at (see, e.g., Prasad, V., Nature 537: S63; 2016). Such low response rates as well as faster drug approvals, e.g., low-value approvals, combined with high prices of such targeted drugs, e.g., $150,000 per patient per year, warrant novel and efficient federal regulations to the use, offer to sell, and/or sale of drugs. In one instance, as to when the clinical trials effectively end, and when the drug approval and commercialization can effectively start can be overlapping; and in some instances, simultaneous, and in some other instances, iterative. In one example, a drug can be approved based on a Phase-2 or a single Phase-3 clinical trial data, and upon commercialization, another Phase-3, longer-term trial can be conducted. In another example, a third-party payer can pay for the Phase-3 clinical trial, e.g., in a non-271(e) clinical trial setting. Such a trial-treatment merger has significant time, cost saving advantages for payers and patients. In one embodiment, the drug cost for payer is cheaper by about 20%, e.g., 30%, 40%, 50%, etc., in the clinical trial. In another instance, such post-approval data can be used to federally regulate the use of drugs, e.g., in stratified subsets of patients. In another instance, such post-approval data can be used to federally regulate the offer to sell and/or sale of drugs, e.g., in defined subsets of patients.

In one instance, reducing the cost of expensive medicines for patients and payers and thereby broadening accessibility and affordability of treatments is a key objective of FDA and other federal agencies such as CMS and DHHS. Analogous counterpart concepts and agencies exist in other countries. For instance, wasteful drug spending can be reduced by identifying those subsets in which a drug works very poorly. In other instances, increasing the quality and therapeutic outcomes of expensive specialty drug treatments, e.g., biologics used to treat autoimmune diseases, is a key objective of federal payers. Several of the healthcare acts in the U.S., e.g., Hatch-Waxman Act, BPCI Act, 21st Century Cures Act, as well as the federal agencies, e.g., FDA, CMS, AHRQ, either directly or indirectly regulate healthcare and the use and sale of drugs, e.g., to achieve safer, higher quality, more accessible, and affordable therapies by developing evidence-based drug treatments, which is of significant value to federal payers, private payers, as well as patients as they pay nearly 20-25% of the drug cost. Analogous counterpart concepts and agencies exist in other countries.

In one embodiment, several FDA-approved drugs achieve excellent response in just 5-20% of treated patients, and nearly 30-70% of patients are poor responders to these therapies. For instance, identifying the excellent responder subset(s) to adalimumab treatment in rheumatoid arthritis is of significant value to FDA and CMS. In another instance, identifying the non-responder subset(s) to adalimumab treatment in rheumatoid arthritis is equally of significant value to CMS. An FDA-mandated or CMS-mandated Phase-4 clinical trial, or a Phase-3 clinical trial can provide such information, which can be submitted to federal agencies, e.g., FDA, CMS. In some instances, this can be an FDA-approved Phase-4 clinical trial, or a Phase-3 clinical trial and the data can be submitted to federal agencies, e.g., FDA, DHHS, and CMS.

In one embodiment, uses reasonably related to the development and submission of information under a federal law which regulates the manufacture, use, or sale of drugs are appropriate to include in a submission of data, e.g., clinical data, to the FDA, FFDCA, or to any other related federal agency, e.g., Centers for Medicare and Medicaid Services (CMS), Department of Health and Human Services (DHHS), Agency for Healthcare Research and Quality (AHRQ). In other instances, the data can include clinical, therapeutic, or economic outcomes on a patient-specific or population-specific basis for a disease indication, e.g., multiple sclerosis, and can be submitted to federal agencies such as FDA and a federal payer, e.g., CMS.

A federal law can regulate the manufacturing of drugs. In one instance, a federal law can regulate the use of drugs. In other instances, a federal law can regulate the offer to sell (marketing), or sale of drugs. In yet another instance, a federal law can regulate the development and submission of any information, e.g., non-routine submission of data or results, regarding manufacturing, use, or sale of drugs. In one embodiment, a federal law that regulates the manufacture, use, or sale of drugs may fall under, without limiting, any one of the healthcare acts in the U.S.: (i) Drug Price Competition and Patent Term Restoration Act of 1984 (also referred to as the Hatch-Waxman Act), which established abbreviated pathways for the approval of drug products under the Federal Food, Drug, and Cosmetic Act (FD&C Act); (ii) Biologics Price Competition and Innovation Act of 2009 (BPCI Act) amends the Public Health Service Act (PHS Act) to create an abbreviated approval pathway for biological products shown to be biosimilar, or interchangeable with, an FDA-licensed reference biological product; (iii) Clinical Laboratory Improvement Amendments Act (CLIA Act 1988); (iv) Healthcare Quality Improvement Act of 1986 (HCQIA); (v) Medicare Act (1945), and Medicare Modernization Act (2003); (vi) Medicaid Act (1965); (vii) Children's Health Insurance Program Act (CHIP Act, 2009) and CHIP Reauthorization Act (2015); (viii) Hospital Readmissions Reduction Program (HRRP Act, 2012); (ix) Health Insurance Portability and Accountability Act (HIPAA Act, 1996); (x) Patient Safety and Quality Improvement Act (PSQIA Act, 2005); (xi) Affordable Care Act of 2010; and (xii) 21st Century Cures Act (2016). Thus, agencies beyond the US FDA may request “development and submission of data” referred to under the patent statute. Analogous counterpart Acts and agencies exist in other countries.

The term “a Federal law” can refer to an entire Act, any Act which regulates the manufacture, sale, or use of drugs. In one instance, the Public Health Services Act (PHSA) regulates the interstate commerce of biologic drugs (42 U.S.C. § 262(a)(1)(A)). Thus, the PHSA regulates the sale of biological products. In one embodiment, the PHSA is a Federal law that regulates the sale of drugs. In another embodiment, the BPCI Act is a Federal law that regulates the use and sale of drugs.

In one embodiment, at least where a third-party clinical trial sponsor, e.g., a managed care company, has a reasonable basis for deducing that a patented compound may or may not work in certain subsets of patients, through a particular biological process or immune mechanism, to produce a particular physiological effect, e.g., remission, excellent clinical response, or alternatively, poor therapeutic outcomes, and uses the compound in clinical research or clinical trial that, if successful, would be appropriate to include in a submission to the FDA, that use is reasonably related to the development and submission of information, e.g., non-routine submission of data or results, under a federal law. In this instance, a third-party sponsor can purchase the drug at an at-cost price from its manufacturer and conduct an FDA-mandated Phase-4 clinical trial, or a Phase-3 clinical trial. In some instances, this can be an FDA-approved Phase-4 clinical trial, or a Phase-3 clinical trial.

In another embodiment, the term drug can be a small molecule, a biologic, e.g., a therapeutic antibody, a therapeutic vaccine, cell therapies, a diagnostic kit, a theragnostic product that includes a cell-based functional immunoassay, a medical device, an implant, or a transplanted organ, e.g., kidney. In one instance, at least where a third-party clinical trial sponsor, e.g., a managed care company, has a reasonable basis for deducing that a drug, e.g., a theragnostic product, may or may not identify certain subsets of patients, through a particular biological process or immune mechanism, to determine a particular clinical or therapeutic effect, e.g., remission, excellent clinical response, or alternatively, poor therapeutic outcomes, and uses the product in clinical research or clinical trial that, if successful, would be appropriate to include in a submission to the FDA, that use is reasonably related to the development and (or) submission of information under a federal law. In this instance, a third-party sponsor can purchase the theragnostic product at an at-cost price from its manufacturer and conduct an FDA-mandated Phase-4 clinical trial, or a Phase-3 clinical trial.

The term sale herein can refer to the process of selling, e.g., selling a drug, which involves explicitly a seller and a buyer in a commercial setting. The term offer to sale herein may refer to marketing. For instances, a seller can be a manufacturer or a drug distributor. For example, a manufacturer can sell a drug directly to a third-party clinical sponsor. In other instances, a manufacturer can sell a drug directly to a third-party payer who is also a clinical trial sponsor. In another instance, a third-party clinical trial sponsor can have provider capabilities, e.g., disease and therapy management, and therefore, a third-party clinical trial sponsor is also a provider. In some other instances, a provider or a laboratory can be the buyer of a drug or a diagnostic product. In yet another instance, a provider can also house a clinical laboratory as per CLIA guidelines, and therefore, a provider is also a laboratory. In yet another instance, a clinical trial sponsor can have both provider and theragnostic capabilities, and therefore, a third-party clinical trial sponsor can function both as a provider and a laboratory.

In one embodiment, the drug referred herein is an FDA-approved, commercially available drug. In one instance, the drug can be sold by a manufacturer at an at-cost price to a buyer. In another instance, a drug can be sold to a third-party sponsor at, e.g., about 5%, 10%, 20%, 40%, 80%, 200%, or more of the at-cost manufacturing price. In another instance, a drug can be bought by a third-party clinical trial sponsor at about 400%, 600%, 800%, or more of the at-cost manufacturing price. In yet other instances, a third-party clinical trial sponsor can buy the drug at 10×, 15×, 30×, 50×, or more of the at-cost manufacturing price from the manufacturer. At-cost manufacturing prices, e.g., for antibodies, can be calculated through a variety of methods including cost of goods sold, net present value calculations, and the like. (see mAbs 1:443-52, 2009). In one instance, per-vial cost can be derived by adding direct and indirect costs associated with manufacturing, production, and processing of biologic drugs. In another instance, to generate the at-cost price at which the drug is sold to a third-party clinical trial sponsor, a manufacturer can add other expenses such as royalties incurred for accessing either the necessary manufacturing and process technologies, antibody engineering technologies, or for the antibody sequence or target, as well as the cost of research and clinical development, sales, and failed projects in the research pipeline, and the like.

### Exemplary Activities Providing Avoidance of Market Exclusivity

Exclusivity is a term used to describe exclusive marketing rights granted by the FDA upon approval of a drug. This can run concurrently whether or not the primary drug patent has expired during this period. It prevents the submission of clinical trial data by generic drug companies, e.g., Abbreviated New Drug Applications (ANDAs) described in Section 505(b)(2) of the US Food, Drug, and Cosmetic Act (FDCA). This exclusivity can be granted upon approval or licensing of a drug product, and the exclusivity period may vary. Five types of exclusivity can include Orphan Drug Exclusivity (ODE) for 7 years; New Chemical Exclusivity (NCE) for 5 years; “Other” for 3 years; Pediatric Exclusivity (PED) for 6 months; and 180-day Exclusivity. A related exclusivity can apply for Qualified Infectious Diseases Products (QIDP) under GAIN. See FDACDER SBIA Chronicles, May 19 2015; www.fda.gov/cdersmallbusinesschronicles. Analogous counterpart exclusivity periods may exist in other countries. Biologics exclusivity can be twelve years.

During this exclusivity period, the FDA may not accept an application for marketing approval. This “market exclusivity” can be distinct from “data exclusivity”. The data exclusivity can be a period during which the FDA may not rely upon an innovator's safety and/or efficacy data for a generic (e.g., small molecule generic or biosimilar) product approvals. However, a generic company can submit independently-generated safety and/or efficacy data, which might be generated outside the jurisdiction, e.g., ex-US.

However, if an application is not submitted for market approval or licensing, the market exclusivity restriction is not violated. See, e.g., Spencer and Adams (August 2014) “Overview of Non-Patent Exclusivities in Major Pharmaceutical Markets” Business Development and Licensing Journal 21:18-23; www.plg-uk.com. In some cases, an exclusivity, which can be, for example, a regulatory exclusivity, a market exclusivity, or a data exclusivity, can be about: 0, 1, 2, 3, 4, 5, 6, 7. 8. 9. 10, 11, 12, 13, 14, 15, 16, 17, 18, 19, 20, 21, 22, or 23 years, and some fraction of a year.

### Databases and Systems

In some cases, clinical trial data obtained by performing clinical trials as described herein can stored on a database. A database can be stored in computer readable format. In some embodiments, clinical trial data can be stored on an electronic storage device on computer readable memory. In some cases, clinical trial data may not be stored on an electronic storage device on computer readable memory. A computer readable memory storing clinical trial data can be comprised in a clinical trial system. In some cases, a clinical trial system can comprise computer readable memory storing clinical trial data and a computer processor. In some cases, a computer processor can be configured to access clinical trial data stored in the computer readable memory.

In some cases, a computer system can be used to analyze data obtained from an assay. FIG. 5 depicts an exemplary system workflow for performing assays for use in a clinical trial. A sample from a patient population can be collected. An assay can be performed as described herein. Assay results can be input into a clinical trial database for diagnostic purposes, for instance, in determining efficacy. A result can be stored remotely or internally on storage medium, and communicated to personnel such as a member of a disease management team, and in some embodiments, a feature may be added which can notify such team member when the results (or series of results) are notable, e.g., indicating abnormal or patient particularly susceptible or resistant to therapy. Such notification may be direct or indirect, but typically can identify a patient to a team member. In some exemplary embodiments, a computer system can compile assay results to determine efficacy of a medicament for a subject. In some cases, a computer system can analyze additional biometric data when determining efficacy. Such additional data can include analyze a subject's age, height, weight, BMI, blood pressure, resting pulse, medical history, mental health status, sex, race, ethnicity, diet, or other risk factors such as smoking, drug or alcohol abuse, or potential drug incompatibilities. In some embodiments, all these data in the storage medium are collected and archived to build a disease-specific data warehouse (FIG. 5), which can be analyzed to discover, analyze disease patterns and treatment response patterns, e.g., in subset(s) of a disease. For instance, in multiple sclerosis or rheumatoid arthritis, such data warehouse may contain, e.g., with 0.5 million patients, 10 million prescriptions (prior authorizations), 20 million lab results, and 10-year longitudinal treatment follow-up. In some embodiments, such a data warehouse is integrated with both clinical trial and conventional treatment platforms (FIG. 2). A warehouse may allow recognition or identification of new subsets of conditions or disease, which may be subject to effective diagnosis and treatment. A warehouse of data may be mined using Artificial Intelligence tools for stratification.

In some cases, a system can be configured with means for transmitting a result. Means for transmitting can include wired and wireless means. Examples of wired communication means can include a Universal Serial Bus (USB) connection, a coaxial cable connection, an Ethernet cable such as a Cat5 or Cat6 cable, a fiber optic cable, or a telephone line. Examples or wireless communication means can include a Wi-Fi receiver, a means for accessing a mobile data standard such as a 3G or 4G LTE data signal, or a Bluetooth receiver. In some cases, data can be transmitted by an email.

In some cases, a system can be configured to communicate with an external database. In some embodiments, a system can transmit data to a database or server. A database or server can be a cloud server or database. In some embodiments, a system can transmit data wirelessly via a Wi-Fi, or Bluetooth connection.

In some aspects, a system described herein can comprise centralized data processing, that could be cloud-based, internet-based, locally accessible network (LAN)-based, or a dedicated reading center using pre-existent or new platforms.

In some aspects, a system can comprise software. A software can rely on structured computation, for example providing registration, segmentation and other functions, with the centrally-processed output made ready for downstream analysis.

In some aspects, the software would rely on unstructured computation, artificial intelligence or deep learning. In a variation of this aspect, the software would rely on unstructured computation, such that data could be iteratively. In a further variation of this aspect, the software can rely on unstructured computation, so-called “artificial intelligence” or “deep learning.” For example, a method described herein such as random forest can employ deep learning to generate Gini impurity scores that can be used to parse out probes with improved predictive value.

The devices can comprise immunoassay devices for measuring profiles of polypeptides or proteins. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. These devices and methods can utilize labeled probes in various sandwiches, competitive or non-competitive assay formats, to generate a signal that can be related to the presence or amount of an analyte of interest. Additionally, certain methods and devices, such as biosensors and optical immunoassays, can be employed to determine the presence or absence of analytes without the need for a labeled molecule. See, e.g., U.S. Pat. Nos. 5,631,171; and 5,955,377, each of which is hereby incorporated by reference in its entirety, including all tables, figures and claims. One skilled in the art can also recognize that robotic instrumentation including but not limited to Beckman ACCESS®, Abbott AXSYM®, Roche ELECSYS®, Dade Behring STRATUS® systems are among the immunoassay analyzers that are capable of performing the immunoassays taught herein.

Computer readable memory can be employed for storing data obtained from a clinical trial described herein, as well as products derived therefrom. A product can include an electronic regulatory submission, a regulatory submission application seeking approval for label revision of a drug, and the like.

In some embodiments, a computer readable memory can store on an electronic storage device an electronic regulatory submission, or a section thereof, in computer readable format, that can contain clinical trial data or a summary thereof obtained from a clinical trial described herein. In some embodiments, a computer readable memory may not store on an electronic storage device an electronic regulatory submission, or a section thereof, in computer readable format, that can contain clinical trial data or a summary thereof obtained from a clinical trial described herein. In some embodiments, computer readable memory can store on an electronic storage device an electronic regulatory submission, or a section thereof, in computer readable format, the electronic regulatory submission containing the steps of for performing a clinical trial as described herein. In some embodiments, computer readable memory may not store on an electronic storage device an electronic regulatory submission, or a section thereof, in computer readable format, the electronic regulatory submission containing the steps of for performing a clinical trial as described herein.

In some embodiments, a computer readable memory can store on an electronic storage device a regulatory submission, or a section thereof, in computer readable format, seeking approval for label revision of a drug that can contain clinical trial data or a summary thereof obtained from a clinical trial described herein. In some embodiments, a computer readable memory may not store on an electronic storage device a regulatory submission, or a section thereof, in computer readable format seeking approval for label revision of a drug, that can contain clinical trial data or a summary thereof obtained from a clinical trial described herein. In some embodiments, computer readable memory can store on an electronic storage device an electronic regulatory submission, or a section thereof, in computer readable format, a regulatory submission seeking approval for label revision of a drug containing the steps of for performing a clinical trial as described herein. In some embodiments, computer readable memory may not store on an electronic storage device a regulatory submission seeking approval for label revision of a drug, or a section thereof, in computer readable format, the electronic regulatory submission containing the steps of for performing a clinical trial as described herein.

In some cases, an electronic drug formulary can be stored, in electronic format, on computer readable memory comprising an electronic storage device. An electronic drug formulary can comprise an electronic database can comprise a listing of medicaments available for administration to a subject. In some cases, an electronic drug formulary database can comprise a non-licensed and non-approved medicament. In some cases, an electronic drug formulary database may not comprise a non-licensed and non-approved medicament. In some cases, an electronic drug formulary database can comprise a licensed and non-approved medicament. In some cases, an electronic drug formulary database may not comprise a licensed and non-approved medicament. In some cases, an electronic drug formulary database can comprise a non-licensed and approved medicament. In some cases, an electronic drug formulary database may not comprise a non-licensed and approved medicament. In some cases, an electronic drug formulary database can comprise a licensed and approved medicament. In some cases, an electronic drug formulary database may not comprise a licensed and approved medicament. In some cases, an electronic drug formulary database can comprise a medicament that can be administered in a clinical trial as described herein. In some cases, an electronic drug formulary database can comprise a medicament that can only be administered in a clinical trial as described herein. In some cases, an electronic drug formulary database can comprise a medicament that may not be administered in a clinical trial as described herein.

Computer readable memory storing an electronic drug formulary can be comprised in a formulary system that can also comprise a computer processor configured to access the electronic drug formulary. Such a system can be configured with communication means as described herein.

An electronic formulary can correspond to a physical storage of medicaments in a pharmacy. In some cases, a pharmacy can comprise a formulary system as described herein and physical storage means storing medicaments. In some embodiments, the physical storage of medicaments and the system are both present in a same building. In some embodiments, the physical storage of medicaments and the system are not present in a same building. In some embodiments, a formulary system can be configured to be accessed in the same building as the physical storage. In some embodiments, a formulary system may not be configured to be accessed in the same building as the physical storage.

A pharmacy can comprise the label revised drug obtained by performing methods described herein present in a container. In some embodiments, a pharmacy can comprise a non-licensed and non-approved medicament. In some embodiments, a pharmacy may not comprise a non-licensed and non-approved medicament. In some embodiments, a pharmacy can comprise a licensed and non-approved medicament. In some embodiments, a pharmacy may not comprise a licensed and non-approved medicament. In some embodiments, a pharmacy can comprise a non-licensed and approved medicament. In some embodiments, a pharmacy may not comprise a non-licensed and approved medicament. In some embodiments, a pharmacy can comprise a licensed and approved medicament. In some embodiments, a pharmacy may not comprise a licensed and approved medicament. In some embodiments, a medicament can be biosimilar to a medicament that can be approved and licensed.

In some embodiments, a pharmacy can comprise a medicament that can have a commercial cost that can be from about 5% to about 100%, from about 10% to about 100%, from about 15% to about 100%, from about 20% to about 100%, from about 25% to about 100%, from about 30% to about 100%, from about 35% to about 100%, from about 40% to about 100%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, or from about 95% to about 100% of a commercial cost of the medicament that can be approved and licensed.

In some embodiments, a pharmacy can comprise a medicament that can be a protein. In some embodiments, a pharmacy can comprise a medicament that may not be a protein. In some embodiments, the pharmacy can comprise a specialty drug. In some embodiments, the pharmacy may not comprise a specialty drug. In some embodiments, a pharmacy can comprise a medicament that can be interchangeable with a medicament that is approved and licensed. In some embodiments, a pharmacy may not comprise a medicament that can be interchangeable with a medicament that is approved and licensed.

In some embodiments, a system as described herein or a component thereof (e.g., a database, a computer network, a formulary, a pharmacy, and the like) may be exempt from having to be cleared, approved or licensed as determined by a regulatory agency, such as the US-FDA, the European Medicines Agency (EMA), the Chinese FDA (China), the Pharmaceuticals and Medical Devices Agency (PMDA) (Japan), or other regulatory agencies as described herein. In some embodiments, a system as described herein or a component thereof (e.g., a database, a computer network, a formulary, a pharmacy, and the like) may have previously been exempted, cleared, approved or licensed as determined by a regulatory agency as described herein. In some embodiments, a system as described herein or a component thereof (e.g., a database, a computer network, a formulary, a pharmacy, and the like) may be a research tool.

A user of a system as described herein or a component thereof (e.g., a database, a computer network, a formulary, a pharmacy, and the like) may be a physician, healthcare worker, case worker, insurer, laboratory technician, or other person typically involved in clinical trials or patient treatment. In some embodiments, a user of a system as described herein or a component thereof (e.g., a database, a computer network, a formulary, a pharmacy, and the like) may not be the sponsor or owner, before a regulatory agency as described herein, of the system or component thereof and/or its architecture and/or hardware.

A user can access a system as described herein or a component thereof (e.g., a database, a computer network, a formulary, a pharmacy, and the like) via an interface. An interface can include peripheral components of a system that allow a user to issue input/output commands, visualize, transmit, and receive data. Such components can include a tablet, a smart phone, a computer, a computer screen, a computer terminal, a computer terminal screen, a keyboard, a router, a wife hotspot, wearable display devices (e.g., smart glasses, which may include virtual reality, augmented reality, or enhanced reality-highlighting features), and the like. For example, a first user can input clinical trial data into a system using a keyboard. The first user can visualize the clinical trial data, e.g., on the computer screen locally connected to the system, and analyze and retrieve data from the database. The first user can transmit the data wirelessly to a second user (e.g., a family member, payer, and the like), who can retrieve the data via a smart phone or a device app. Such components can be used to transmit or display data from the methods, clinical trials, and/or systems as described herein, in whole or in part (e.g., comprising at least some of the data).

### Computer-Implemented Methods of Selection

In some embodiments, a diagnostic and/or theragnostic as described herein can be used to select a subject for clinical trial in combination with an electronic formulary as described herein. In some embodiments, a subject can be selected for a clinical trial or a conventional treatment, by: (a) consulting a drug formulary system as described herein to access a formulary; (b) performing a diagnostic or theragnostic test such as an assay on a sample from the subject, or obtaining data derived therefrom, to determine a disease state; and (c) providing a prior authorization, wherein the prior authorization comprises: enrolling the subject in the clinical trial to receive a non-licensed and non-approved medicament recited in the formulary from a pharmacy; or providing a conventional treatment, wherein the subject does not participate in the clinical trial; where the prior authorization can be provided based on the assay or data derived therefrom.

In some embodiments, a subject may not be selected for a clinical trial or a conventional treatment by consulting a drug formulary system as described herein to access a formulary. In some embodiments, a subject may not be selected for a clinical trial or a conventional treatment by performing a diagnostic or theragnostic test such as an assay on a sample from the subject, or obtaining data derived therefrom. In some embodiments, a subject may not be selected for a clinical trial or a conventional treatment by providing a prior authorization. In some embodiments, a prior authorization may not comprise enrolling the subject in the clinical trial to receive a non-licensed and non-approved medicament recited in the formulary from a pharmacy. In some embodiments, a prior authorization may not comprise providing a conventional treatment, wherein the subject does not participate in the clinical trial. In some embodiments, a prior authorization may not be based on the assay or data derived therefrom.

In some embodiments, a medicament to be administered to a subject can be determined by employing a clinical trial system as described herein. In some embodiments, a medicament can be administered by consulting a clinical trial system as described herein and selecting a medicament to be administered based on a progression of treatment. In some embodiments, a medicament may not be administered by consulting a clinical trial system as described herein. In some embodiments, a medicament may not be administered by based on a progression of treatment.

In some embodiments, a clinical trial provider who can be at a different location than the subject can consult the clinical trial system. In some embodiments, a clinical trial provider who can be at the same location as the subject can consult the clinical trial system.

In some embodiments, a medicament can be administered after consulting a clinical trial database as described herein. In some embodiments, a specialty distributor may be involved in the process of administering to the subject. In some embodiments, a specialty distributor may not be involved in the process of administering to the subject. In some embodiments, a pharmacy benefit manager (PBM) may be involved in the process of administering to the subject. In some embodiments, a pharmacy benefit manager (PBM) may not be involved in the process of administering to the subject.

In some embodiments, a method can comprise entering data obtained from the clinical trial into a clinical trial system. In some embodiments, a method may not comprise entering data obtained from the clinical trial into a clinical trial system. In some embodiments, a clinical trial system can comprise a computer readable memory storing on an electronic storage device a database comprising the clinical trial data, records therefrom, or a summary thereof, in computer readable format. In some embodiments, a clinical trial system may not comprise a computer readable memory storing on an electronic storage device a database comprising the clinical trial data, or a summary thereof, in computer readable format. In some embodiments, a clinical trial system can comprise a computer processor, where the computer processor can be configured to access clinical trial data from the computer readable memory. In some embodiments, a clinical trial system can comprise a computer processor, where the computer processor may not be configured to access clinical trial data from the computer readable memory. A “computer processor” can include a microcontroller and/or a microprocessor, and can contain an integrated CPU processor core, an arithmetic and logic unit, a register, an internal clock, an internal bus, a logic gate, a transistor, a ceramic cover, computer memory, program memory, and/or a programmable input/output peripheral. In some cases, program memory can include ferroelectric RAM, NOR flash, OTP ROM, or RAM. HIPAA compliant procedures, suitable encryption and security precautions can be incorporated to protect data privacy.

### Slowing Progression of Disease

In some embodiment, performing a clinical trial as described herein can be used to slow the progression of a disease or condition as described herein. In some embodiment, employing a clinical trial database as described herein can be used to slow the progression of a disease or condition as described herein. In some embodiment, a clinical trial system as described herein can be used to slow the progression of a disease or condition as described herein.

An exemplary method can include slowing the progression of rheumatoid arthritis. In some embodiments, a method can comprise: separating the subject into a subject pool subset based on a selection from the group consisting of: IgG RF^{+/-}, IgG ACPA^{+/-}, IgA RF^{+/-}, IgA ACPA^{+/-}, CRP^{Hi/Lo}, cFib^{+/-}; and FcGR-3A, FcGR-2A, FcGR-3B, and FcAR polymorphisms. In some embodiments, a method may not comprise: separating the subject into a subject pool subset based on a selection from the group consisting of: IgG RF^{+/-}, IgG ACPA^{+/-}, IgA RF^{+/-}, IgA ACPA^{+/-}, CRP^{Hi/Lo}, cFib^{+/-}; and FcGR-3A ¹⁵⁸VF, FcGR-2A ¹³¹HR FcGR-3B, and FcAR polymorphisms. In some embodiments, a method can comprise administering a medicament to the subject, where the medicament can be a non-licensed and non-approved drug that is a biosimilar to a licensed and approved drug. In some embodiments, a method may not comprise administering a medicament to the subject, where the medicament can be a non-licensed and non-approved drug that is biosimilar to a licensed and approved drug. In some embodiments, a method can comprise administering an additional medicament to the subject if the subject does not achieve remission after a time period of about 1 year. In some embodiments, a method may not comprise administering an additional medicament to the subject if the subject does not achieve remission after a time period of about 1 year. In some embodiments, an additional medicament can be a non-licensed and non-approved drug that is biosimilar to a licensed and approved drug. In some embodiments, an additional medicament may not be a non-licensed and non-approved drug that is biosimilar to a licensed and approved drug. In some embodiments, an additional medicament can be the same as the medicament. In some embodiments, an additional medicament can be different from the medicament.

In some embodiments, a method can be employed to treat a patient population using either a clinical trial or a conventional treatment. In some embodiments, a method can comprise obtaining a subject pool comprising subjects having or suspected of having a disease or condition. In some embodiments, a method may not comprise obtaining a subject pool comprising subjects having or suspected of having a disease or condition. In some embodiments, a subject pool can be referred by a payer. In some embodiments, a subject pool may not be referred by a payer. In some embodiments, a method can comprise compiling a formulary. In some embodiments, a method may not comprise compiling a formulary. In some embodiments, a formulary can comprise approved or licensed medicaments. In some embodiments, a formulary may not comprise approved or licensed medicaments. In some embodiments, a formulary can comprise non-approved or non-licensed medicaments. In some embodiments, a formulary may not comprise non-approved or non-licensed medicaments. In some embodiments, a method can comprise storing the formulary in electronic format into a formulary system that comprises a computer readable memory configured to store the formulary on an electronic storage device. In some embodiments, a method may not comprise storing the formulary in electronic format into a formulary system that comprises a computer readable memory configured to store the formulary on an electronic storage device. In some embodiments, a method can comprise stratifying the subject pool into subjects who can enroll in a clinical trial or subject who can receive a conventional treatment paradigm. In some embodiments, a method may not comprise stratifying the subject pool into subjects who can enroll in a clinical trial or subject who can receive a conventional treatment paradigm. In some embodiments, stratifying can comprise consulting the formulary system to access the formulary. In some embodiments, stratifying may not comprise consulting the formulary system to access the formulary. In some embodiments, stratifying can comprise performing an assay on samples from subjects, or obtaining data derived therefrom, to determine a disease state genotype, phenotype, or immunological feature. In some embodiments, stratifying may not comprise performing an assay on samples from subjects, or obtaining data derived therefrom, to determine a disease state genotype, phenotype, or immunological feature. In some embodiments, stratifying can comprise providing a prior authorization. In some embodiments, stratifying may not comprise providing a prior authorization. In some embodiments, prior authorization can be provided based on the assay of data derived therefrom. In some embodiments, prior authorization may not be provided based on the assay of data derived therefrom. In some embodiments, prior authorization can comprise enrolling subjects in the clinical trial to receive a non-licensed and non-approved medicament from the formulary. In some embodiments, prior authorization may not comprise enrolling subjects in the clinical trial to receive a non-licensed and non-approved medicament from the formulary. In some embodiments, prior authorization can comprise providing a conventional treatment from the formulary, wherein the subjects do not participate in the clinical trial. In some embodiments, prior authorization may not comprise providing a conventional treatment from the formulary, wherein the subjects do not participate in the clinical trial. In some embodiments, a method can comprise administering a non-licensed and non-approved medicament to subjects enrolled in the clinical trial. In some embodiments, a method may not comprise administering a non-licensed and non-approved medicament to subjects enrolled in the clinical trial. In some embodiments, a method can comprise administering an additional medicament to the subject if the subjects that do not achieve remission after a time period of about 1 year; where the additional medicament can be a non-licensed and non-approved drug that may not be the same as the medicament. In some embodiments, a method may not comprise administering an additional medicament to the subject if the subjects that do not achieve remission after a time period of about 1 year; where the additional medicament can be a non-licensed and non-approved drug that may not be the same as the medicament.

A system can be used to administer treatment to a subject. In some cases, a system can comprise a pharmacy and a formulary, where the system can be employed to treat a population of subjects comprising a first and a second plurality of subjects by at least two different treatment paradigms. In some cases, a system may not comprise a pharmacy and a formulary. In some cases, a first plurality of subjects can be enrolled in a first treatment paradigm and a second plurality of subjects can be enrolled in a second treatment paradigm. In some embodiments, a first treatment paradigm can comprise administering a licensed, approved, or licensed and approved medicament to treat a disease or condition indicated for the licensed, the approved, or the licensed and approved medicament. In some embodiments, a first treatment paradigm may not comprise administering a licensed, approved, or licensed and approved medicament to treat a disease or condition indicated for the licensed, the approved, or the licensed and approved medicament. In some embodiments, a second treatment paradigm can comprise administering an unlicensed and unapproved medicament to treat the same disease or condition, where the unlicensed and unapproved medicament can be administered with an assurance to a payer based on efficacy, e.g., on a subject-by-subject basis. In some embodiments, a second treatment paradigm may not comprise administering an unlicensed and unapproved medicament to treat the same disease or condition, where the unlicensed and unapproved medicament can be administered with an assurance to a payer based on efficacy, e.g., on a subject-by-subject basis. In some embodiments, a first and second plurality of subjects are different. In some embodiments, a first and second plurality of subjects are the same. In some embodiments, a pharmacy can comprise unlicensed; unapproved; approved; licensed; and licensed and approved medicaments.

Employing clinical trials can be a benefit to society. By employing methods described herein, safety, efficacy, or safety and efficacy of medicaments can be increased through prolonged clinical trials and through successive iterations of clinical trial data that can be generated.

TABLE 4
Exemplary Embodiments

A1. A clinical trial testing a drug, wherein said trial can be funded at least in part by a
third-party payer, which may be a healthcare payer or insurer, and wherein said trial may
not be substantially funded by a pharmaceutical company that developed
and/or commercialized said drug.
A2. A clinical trial comprising a sizeable number of subjects, wherein said subjects may
pay to participate in the trial.
A3. The trial of embodiment A2, wherein at least a sizeable number of said subjects may
directly pay-to-participate, or indirectly pay-to-participate, e.g., through an insurer or other
payer.
A4. The trial of embodiment A2, wherein:
a) said trial can be a single-site trial, or can be a virtual-site trial; and/or
b) said trial can include at least some subjects who are provided assurance, which may be a
therapeutic and/or financial assurance.
A5. The trial of embodiment A2, wherein said trial:
a) can be funded, wholly or at least in part, by a third-party payer, such as a private payer,
government payer, pension fund, employer, or managed care company, but may not be
substantially funded by a pharmaceutical company that developed and/or commercialized
said drug;
b) can be large, such as thousands or tens of thousands of subjects;
c) can be conducted for a substantial period, such as at least about 4, 6, 7, 8, 9, or more
years;
d) can be a phase-4 clinical trial; and/or
e) can be for testing a drug or equivalent listed in table 1, 2, or 3.
B1. A method of conducting a clinical trial of substantial size, wherein at least a sizeable
number of subjects may enroll on a pay-to-participate basis.
B2. The method of embodiment B1, wherein said sizeable number of subjects may pay-to-
participate either directly or their cost can be borne wholly or at least in part by a third-
party payer.
B3. The method of embodiment B1, wherein:
a) said trial can be a single-site trial, or can be a virtual-site trial; and/or
b) said trial can include at least some subjects who are provided assurance, which can be a
therapeutic and/or financial assurance.
B4. The method of embodiment B1, wherein said trial:
a) can be conducted, wholly or at least in part, by a third-party sponsor, such as a managed
care company or a fully-integrated payer having managed care capabilities, but may not be
funded by a pharmaceutical company that developed and/or commercialized the drug that
is being tested;
b) can be large, such as thousands or tens of thousands of subjects;
c) can be conducted for a substantial period, such as at least about 4, 6, 7, 8, 9, or more
years;
d) can be a phase-4 clinical trial; and/or
e) can be for testing a drug or equivalent which is listed in table 1, 2, or 3.
C1. A clinical trial testing a drug, wherein said trial can be conducted by a third-party
sponsor, and said third-party sponsor cannot be a pharmaceutical company that developed,
and/or commercialized said drug.
C2. The trial of embodiment C1, wherein said third-party sponsor can be a managed care
company.
C3. The trial of embodiment C1, wherein:
a) said trial can be a single-site trial, or can be a virtual-site trial; or
b) said trial can include at least some subjects who are provided assurance, which can be a
therapeutic and/or financial assurance.
C4. The trial of embodiment C2, wherein said trial:
a) can be funded, wholly or at least in part, by a payer, an employer, or managed care
company, or wholly or in part by pay-to-participate subjects, whose payments can cover
full or partial drug and/or participation costs;
b) can be sizeable, such as thousands or tens of thousands of subjects;
c) can be conducted for a substantial period, such as at least about 4, 6, 7, 8, 9, or more
years;
d) can be a phase-4 clinical trial; and/or
e) can be for testing a drug or equivalent which is listed in table 1, 2, or 3.
D1. A method of conducting a clinical trial by a third-party sponsor.
D2. The method of embodiment D1, wherein said third-party sponsor can be a managed
care company, or a healthcare payer having managed care capabilities.
D3. The method of embodiment D1, wherein:
a) said trial can be a single-site trial, or can be a virtual-site trial; and/or
b) said trial can include at least some subjects who are provided assurance, which can be a
therapeutic and/or financial assurance.
D4. The method of embodiment D1, wherein said trial:
a) either can be funded, wholly or at least in part, by pay-to-participate subjects whose
payments can cover full or partial drug and/or participation costs; or may be paid, wholly
or at least in part, by a payer such as an insurer, employer, or a managed care company;
b) can be sizeable, such as thousands or tens of thousands of subjects;
c) can be conducted for a substantial period, such as at least about 4, 6, 7, 8, 9, or more
years;
d) can be a phase-4 clinical trial; and/or
e) can be for testing a drug or equivalent which is listed in table 1, 2, or 3.
E1. A method of gaining exemption from infringement of a national patent with claims
covering a patented technology, which can comprise using a patented technology solely for
uses reasonably related to the development or submission of information under a Federal
law which regulates the manufacture, use, offers to sell, or sale of drugs.
E2. The method of embodiment El, wherein the uses reasonably related to the development
or submission of information under a Federal law which regulates the manufacture, use,
offers to sell, or sale of drugs can be a clinical trial.
E3. The method of embodiment El, wherein said national patent can be issued in the US,
Europe, UK, Germany, France, Spain, Italy, Sweden, Canada, Mexico, Japan, China, South
Korea, or India.
E4. The method of embodiment E2, wherein:
a) said trial can be a single-site trial, or can be a virtual-site trial; and/or
b) said trial can include at least some subjects who are provided assurance, which can be a
therapeutic and/or financial assurance.
E5. The trial of embodiment E2, wherein said trial:
a) can be funded, wholly or at least in part, by a third-party payer, an employer, or a
managed care company; or can include pay-to-participate subjects whose payments can
cover full or partial drug and/or participation costs;
b) can be conducted by a third-party sponsor;
c) can be sizeable, such as thousands or tens of thousands of subjects;
d) can be conducted for a substantial period, such as at least about 4, 6, 7, 8, 9, or more
years;
e) can be a phase-4 clinical trial; and/or
f) can be for testing a drug or equivalent which is listed in table 1, 2, or 3.
F1. A method of administering a drug to patients in a clinical trial, wherein said drug is
covered by market exclusivity.
F2. The method of Claim F1, wherein the market exclusivity exists in the US, Europe, EP,
UK, Germany, France, Spain, Italy, Sweden, Canada, Mexico, Japan, China, South Korea,
or India.
F3. The method of Claim F1, wherein said trial:
a) is funded, wholly or at least in part, by a third-party payer, an employer, or a managed
care company; or includes pay-to-participate subjects whose payments cover full or partial
drug and/or participation costs;
b) is conducted by a third-party sponsor;
c) is sizeable, such as hundreds or thousands or tens of thousands of subjects; and/or
d) is conducted for a substantial period, such as at least about 4, 6, 7, 8, 9, or more years.
F4. The method of Claim F1, wherein:
a) said trial is a single-site trial, or is a virtual-site trial; and/or
b) said trial includes at least some subjects who are provided assurance, which is a
therapeutic and/or financial assurance.
F5. The trial of Claim F1, wherein said trial:
a) is a phase-2 or -3 clinical trial; and/or
b) is testing a drug or equivalent which is listed in table 1, 2, or 3.
In some embodiments, a study or clinical trial can test a drug, wherein the trial can be
funded, wholly or at least in part, by a third-party payer, which may be a healthcare payer
or insurer, and the trial typically may not be substantially funded by a pharmaceutical
company that developed and/or commercialized said drug. Trials are conceived with any or
all combinations of features described.
In some embodiments, a clinical trial can comprise a sizeable number of pay-to-
participate subjects. In certain embodiments, the trial can comprises at least a sizeable
number of subjects who directly pay-to-participate, or indirectly pay-to-participate, e.g.,
through an insurer or other payer. In some embodiments, the trial can be a single-site trial,
or can be a virtual-site trial; and/or can includes at least some subjects who are provided
assurance, which can be therapeutic and/or financial. In some embodiments, the trial (in all
various combinations): can be funded, wholly or at least in part, by a third-party payer,
such as a private payer, government payer, pension fund, employer, or managed care
company, but not substantially by a pharmaceutical company that develops and/or
commercializes drugs; can be large, such as thousands or tens of thousands of subjects; can
be conducted for a substantial period, such as at least about 4, 6, 7, 8, 9, or more years; can
be a phase-2, a phase-3, a merged, or a phase-4 clinical trial; can provide exemption from
patent infringement, including from an “evergreening” patent; and/or can be testing a drug
or equivalent listed in table 1, 2, or 3, e.g., adalimumab. In some embodiments, the trial can
include one or more subjects, or take place wholly or in part, in the US, Europe, EP, UK,
Germany, France, Spain, Italy, Sweden, Canada, Mexico, Japan, China, South Korea,
or India. In other embodiments, the study or trial is directed to a product covered by a patent
includes one or more claims which is directed to: provide an “evergreening” patent
coverage, including subject matter directed to any of: a method of manufacturing; a new
method of manufacturing an existing drug (or structural similar, salt, polymorph, solvate,
biosimilar, or chemical or metabolic conversion structure, or the like); a new target patient
group for an existing drug (or structural similar, salt, polymorph, solvate, biosimilar, or
chemical or metabolic conversion structure); a new means to use or administer an existing
drug (or structural similar, salt, polymorph, solvate, biosimilar, or chemical or metabolic
conversion structure, or the like), including with use of a companion diagnostic; a new
indication for an existing drug (or structural similar, salt, polymorph, solvate, biosimilar, or
chemical or metabolic conversion structure, or the like); a combination composition
comprising an existing drug; a new use for an existing drug (or structural similar, salt,
polymorph, solvate, biosimilar, or chemical or metabolic conversion structure, or the like)
composition; a variant isomer of an existing drug (or structural similar, salt, polymorph,
solvate, biosimilar, or chemical or metabolic conversion structure, or the like); a new
formulation for an existing drug (or structural similar, salt, polymorph, solvate, biosimilar,
or chemical or metabolic conversion structure, or the like); a new route of administration
for an existing drug (or structural similar, salt, polymorph, solvate, biosimilar, or chemical
or metabolic conversion structure, or the like); a drug (or structural similar, salt,
polymorph, solvate, biosimilar, or chemical or metabolic conversion structure, or the like)
or use thereof which had been initially approved (either in US, or elsewhere) a time (e.g., at
least about an integral number of years, like 1, 2, 3, 4, 5, 6, 7, 8, 9, 10, 11, 12, 13, 14, 15,
16, 17, 18, 19, 20, 21, 22, etc., and a fraction such as .00, .05, .10, .15, .20, .25, .30, .35,
.40, .45, .50, .55, .60, .65, .70, .75, .80, .85, .90, or .95) or more prior; a drug (or structural
similar, salt, polymorph, solvate, biosimilar, or chemical or metabolic conversion structure,
or the like) or use thereof, first sold, in the US or elsewhere, a period (e.g., at least about an
integral number of years, like 1, 2, 3, 4, 5, 6, 7, 8, 9, 10, 11, 12, 13, 14, 15, 16, 17, 18, 19,
20, 21, 22, 23, 24, 25, etc., plus a fraction such as .00, .05, .10, .15, .20, .25, .30, .35, .40,
.45, .50, .55, .60, .65, .70, .75, .80, .85, .90, or .95) or more prior; a drug or use thereof, for
which market approval was based on reference to human clinical studies (e.g., phase-1,
safety, or human testing, e.g., phase-2, or phase 3) initiated a term (e.g., at least about an
integral number of years, like 1, 2, 3, 4, 5, 6, 7, 8, 9, 10, 11, 12, 13, 14, 15, 16, 17, 18, 19,
20, 21, 22, 23, 24, 25, etc., plus a fraction such as .00, .05, .10, .15, .20, .25, .30, .35, .40,
.45, .50, .55, .60, .65, .70, .75, .80, .85, .90, or .95) or more prior; and/or a substitute or
replacement part of an implant or device. Databases, processing systems, procedures, and
operations comprising or handling records including data from such studies are also
conceived. Businesses and business methods comprising the described studies are also
provided.
In other embodiments are conceived a subject (and/or data therefrom) participating in the
study, e.g., one who pays to participate. The subject may be an adult, child, senior,
disabled, having an orphan disease, or the like, and may be covered (or used) by health
insurance provided by, e.g., CMS, VA, Medicare, private insurance, self-insured,
supplemental Medicare, or some combination. In addition, providers serving such subject,
e.g., a doctor, healthcare provider, healthcare assistant, clinical study or trial administrator,
or other individual or entity (e.g., medical practice, medical center, hospital, private or
public chain or consortium) which assists in the study on the subject, whether the subject is
in an empirical or control arm; or a pharmacy, pharmacist, PBM, diagnostic laboratory, or
other entity which supplies drug, device, testing, or treatment materials to said subject, e.g.,
as part of or in the course of the study; or a study sponsor, employee thereof, advisor,
agent, consultant, or the like who is involved in or assists in the design of the study
generating the data, e.g., medical data, collected by the study, whether the patient is located
in the same or a foreign jurisdiction; and an insurance person, employee, agent, consultant,
administrator, bureaucrat, or the like who handles, processes, or approves authorization for
treatment or payment for said subject, are conceived. Use of study data by sponsor is also
covered, e.g., to subset patients or diseases/conditions, but most importantly,
responsiveness to treatment. The drugs, devices, companion diagnostics, related products,
and the like, and of methods used in or pursuant to said study, and those who supply or
perform them, are also covered herein.
Another aspect provides a method of conducting a study or clinical trial of substantial size,
wherein all or at least a sizeable number of subjects enroll on a pay-to-participate
basis. Methods are conceived with any and all combinations of features described. In some
embodiments, the sizeable number of subjects pay-to-participate either directly or their cost
can be borne at least in part by a third-party payer. In some embodiments, the trial is a
single-site trial, or can be a virtual-site trial; and/or the trial includes at least some subjects
who are provided assurance, which can be therapeutic and/or financial. In some
embodiments, the trial (in all various combinations): can be conducted at least in part by a
third-party sponsor, such as a managed care company or a fully-integrated payer having
managed care capabilities, but typically not a pharmaceutical company that developed
and/or commercialized the drug being tested; can be large, such as thousands or tens of
thousands of subjects; can be conducted for a substantial period, such as at least about 4, 6,
7, 8, 9, or more years; can be a phase-2, a phase-3, a merged, or a phase-4 clinical trial; can
provide exemption from patent infringement, including from an “evergreening” patent;
and/or can be testing a drug or equivalent which is listed in table 1, 2, or 3, e.g.,
adalimumab. In some embodiments, the study may include one or more subjects, or take
place wholly or in part, in the US, Europe, EP, UK, Germany, France, Spain, Italy,
Sweden, Canada, Mexico, Japan, China, South Korea, or India. In other embodiments, the
method is directed to a research study or trial on a drug which is covered by a patent which
includes one or more claims which is directed to: provide an “evergreening” patent
coverage, including subject matter directed to any of: a method of manufacturing; a new
method of manufacturing an existing drug (or structural similar, salt, polymorph, solvate,
biosimilar, or chemical or metabolic conversion structure, or the like); a new target patient
group for an existing drug (or structural similar, salt, polymorph, solvate, biosimilar, or
chemical or metabolic conversion structure, or the like); a new means to use or administer
an existing drug (or structural similar, salt, polymorph, solvate, biosimilar, or chemical or
metabolic conversion structure, or the like), including with use of a companion diagnostic;
a new indication for an existing drug (or structural similar, salt, polymorph, solvate,
biosimilar, or chemical or metabolic conversion structure, or the like); a combination
composition comprising an existing drug; a new use for an existing drug (or structural
similar, salt, polymorph, solvate, biosimilar, or chemical or metabolic conversion structure,
or the like) composition; a variant isomer of an existing drug (or structural similar, salt,
polymorph, solvate, biosimilar, or chemical or metabolic conversion structure, or the like);
a new formulation for an existing drug (or structural similar, salt, polymorph, solvate,
biosimilar, or chemical or metabolic conversion structure, or the like); a new route of
administration for an existing drug (or structural similar, salt, polymorph, solvate,
biosimilar, or chemical or metabolic conversion structure, or the like); a drug (or structural
similar, salt, polymorph, solvate, biosimilar, or chemical or metabolic conversion structure,
or the like) or use thereof which had been initially approved (either in US, or elsewhere) a
time (e.g., at least about an integral number of years, like 1, 2, 3, 4, 5, 6, 7, 8, 9, 10, 11, 12,
13, 14, 15, 16, 17, 18, 19, 20, 21, 22, etc., plus a fraction such as .00, .05, .10, .15, .20, .25,
.30, .35, .40, .45, .50, .55, .60, .65, .70, .75, .80, .85, .90, or .95) or more prior; a drug (or
structural similar, salt, polymorph, solvate, biosimilar, or chemical or metabolic conversion
structure, or the like) or use thereof, first sold, in the US or elsewhere, a period (e.g., at
least about an integral number of years, like 1, 2, 3, 4, 5, 6, 7, 8, 9, 10, 11, 12, 13, 14, 15,
16, 17, 18, 19, 20, 21, 22, 23, 24, 25, etc., plus a fraction such as .00, .05, .10, .15, .20, .25,
.30, .35, .40, .45, .50, .55, .60, .65, .70, .75, .80, .85, .90, or .95) or more prior; a drug or use
thereof, for which market approval was based on reference to human clinical studies (e.g.,
phase-1, safety, or human testing, e.g., phase-2, or phase 3) initiated a term (e.g., at least
about an integral number of years, like 1, 2, 3, 4, 5, 6, 7, 8, 9, 10, 11, 12, 13, 14, 15, 16, 17,
18, 19, 20, 21, 22, 23, 24, 25, etc., plus a fraction such as .00, .05, .10, .15, .20, .25, .30,
.35, .40, .45, .50, .55, .60, .65, .70, .75, .80, .85, .90, or .95) or more prior; and/or a
substitute or replacement part of an implant or device. Databases, processing systems,
procedures, and operations comprising or handling records including data from such
methods are also conceived. Businesses and business methods using the described methods
are provided.
Another aspect provides a study or clinical trial testing a drug, wherein the study or trial
can be conducted by a third-party sponsor, and the third-party sponsor typically may not be
a pharmaceutical company that developed, and/or commercialized the drug. Trials are
conceived with any or all combinations of features described. In some embodiments, the
third-party sponsor can be a managed care company. In some embodiments, the trial can be
a single-site trial, or can be a virtual-site trial; and/or the trial includes at least some
subjects who are provided assurance, which can be therapeutic and/or financial. Another
aspect provides such a trial which (in all various combinations): can be funded, at least in
part, by a payer, an employer, or managed care company, or in part by pay-to-participate
subjects, whose payments cover at least some drug and/or participation costs; can be
sizeable, such as thousands or tens of thousands of subjects; can be conducted for a
substantial period, such as at least about 4, 6, 7, 8, 9, or more years; can be a phase-2,
a phase-3, a merged, or a phase-4 clinical trial; can provide exemption from patent
infringement, including from an “evergreening” patent; and/or can be testing a drug or
equivalent which is listed in table 1, 2, or 3. In other embodiments, the study or trial is
directed to a drug which is covered by a patent which includes one or more claims which is
directed to: provide an “evergreening” patent coverage, including subject matter directed to
any of: a method of manufacturing; a new method of manufacturing an existing drug (or
structural similar, salt, polymorph, solvate, biosimilar, or chemical or metabolic conversion
structure, or the like); a new target patient group for an existing drug (or structural similar,
salt, polymorph, solvate, biosimilar, or chemical or metabolic conversion structure, or the
like); a new means to use or administer an existing drug (or structural similar, salt,
polymorph, solvate, biosimilar, or chemical or metabolic conversion structure, or the like),
including with use of a companion diagnostic; a new indication for an existing drug (or
structural similar, salt, polymorph, solvate, biosimilar, or chemical or metabolic conversion
structure, or the like); a combination composition comprising an existing drug; a new use
for an existing drug (or structural similar, salt, polymorph, solvate, biosimilar, or chemical
or metabolic conversion structure, or the like) composition; a variant isomer of an existing
drug (or structural similar, salt, polymorph, solvate, biosimilar, or chemical or metabolic
conversion structure, or the like); a new formulation for an existing drug (or structural
similar, salt, polymorph, solvate, biosimilar, or chemical or metabolic conversion structure,
or the like); a new route of administration for an existing drug (or structural similar, salt,
polymorph, solvate, biosimilar, or chemical or metabolic conversion structure, or the like);
a drug (or structural similar, salt, polymorph, solvate, biosimilar, or chemical or metabolic
conversion structure, or the like) or use thereof which had been initially approved (either in
US, or elsewhere) a time (e.g., at least about an integral number of years, like 1, 2, 3, 4, 5,
6, 7, 8, 9, 10, 11, 12, 13, 14, 15, 16, 17, 18, 19, 20, 21, 22, etc., plus a fraction such as .00,
.05, .10, .15, .20, .25, .30, .35, .40, .45, .50, .55, .60, .65, .70, .75, .80, .85, .90, or .95) or
more prior; a drug (or structural similar, salt, polymorph, solvate, biosimilar, or chemical
or metabolic conversion structure, or the like) or use thereof, first sold, in the US or
elsewhere, a period (e.g., at least about an integral number of years, like 1, 2, 3, 4, 5, 6, 7,
8, 9, 10, 11, 12, 13, 14, 15, 16, 17, 18, 19, 20, 21, 22, 23, 24, 25, etc., plus a fraction such
as .00, .05, .10, .15, .20, .25, .30, .35, .40, .45, .50, .55, .60, .65, .70, .75, .80, .85, .90, or
.95) or more prior; a drug or use thereof, for which market approval was based on reference
to human clinical studies (e.g., phase-1, safety, or human testing, e.g., phase-2, or phase 3)
initiated a term (e.g., at least about an integral number of years, like 1, 2, 3, 4, 5, 6, 7, 8, 9,
10, 11, 12, 13, 14, 15, 16, 17, 18, 19, 20, 21, 22, 23, 24, 25, etc., plus a fraction such as .00,
.05, .10, .15, .20, .25, .30, .35, .40, .45, .50, .55, .60, .65, .70, .75, .80, .85, .90, or .95) or
more prior; and/or a substitute or replacement part of an implant or device. Databases,
processing systems, procedures, and operations comprising or handling records including
data from such studies are also conceived.
Another aspect provides a method of conducting a study or clinical trial by a third-party
sponsor. Methods are conceived with any and all combinations of features described. In
some embodiments, the third-party sponsor can be a managed care company, or a
healthcare payer having managed care capabilities. In certain embodiments of the method,
the trial can be a single-site trial, or can be a virtual-site trial; and/or the trial includes at
least some subjects who are provided assurance, which can be therapeutic and/or financial.
In some aspects of the methods, the trial (in all various combinations): can be funded, at
least in part, by pay-to-participate subjects, whose payments cover some drug and/or
participation costs, which may be paid at least in part by a payer such as an insurer,
employer, or a managed care company; can be sizeable, such as thousands or tens of
thousands of subjects; can be conducted for a substantial period, such as at least about 4, 6,
7, 8, 9, or more years; can be a phase-2, a phase-3, a merged, or a phase-4 clinical trial; can
provide exemption from patent infringement, including from an “evergreening” patent;
and/or can be testing a drug or equivalent which is listed in table 1, 2, or 3. In other
embodiments, the study or trial is directed to a drug which is covered by a patent which
includes one or more claims which is directed to: provide an “evergreening” patent
coverage, including subject matter directed to any of: a method of manufacturing; a new
method of manufacturing an existing drug (or structural similar, salt, polymorph, solvate,
biosimilar, or chemical or metabolic conversion structure, or the like); a new target patient
group for an existing drug (or structural similar, salt, polymorph, solvate, biosimilar, or
chemical or metabolic conversion structure, or the like); a new means to use or administer
an existing drug (or structural similar, salt, polymorph, solvate, biosimilar, or chemical or
metabolic conversion structure, or the like), including with use of a companion diagnostic;
a new indication for an existing drug (or structural similar, salt, polymorph, solvate,
biosimilar, or chemical or metabolic conversion structure, or the like); a combination
composition comprising an existing drug; a new use for an existing drug (or structural
similar, salt, polymorph, solvate, biosimilar, or chemical or metabolic conversion structure,
or the like) composition; a variant isomer of an existing drug (or structural similar, salt,
polymorph, solvate, biosimilar, or chemical or metabolic conversion structure, or the like);
a new formulation for an existing drug (or structural similar, salt, polymorph, solvate,
biosimilar, or chemical or metabolic conversion structure, or the like); a new route
of administration for an existing drug (or structural similar, salt, polymorph, solvate,
biosimilar, or chemical or metabolic conversion structure, or the like); a drug (or structural
similar, salt, polymorph, solvate, biosimilar, or chemical or metabolic conversion structure,
or the like) or use thereof which had been initially approved (either in US, or elsewhere) a
time (e.g., at least about an integral number of years, like 1, 2, 3, 4, 5, 6, 7, 8, 9, 10, 11, 12,
13, 14, 15, 16, 17, 18, 19, 20, 21, 22, etc., plus a fraction such as .00, .05, .10, .15, .20, .25,
.30, .35, .40, .45, .50, .55, .60, .65, .70, .75, .80, .85, .90, or .95) or more prior; a drug (or
structural similar, salt, polymorph, solvate, biosimilar, or chemical or metabolic conversion
structure, or the like) or use thereof, first sold, in the US or elsewhere, a period (e.g., at
least about an integral number of years, like 1, 2, 3, 4, 5, 6, 7, 8, 9, 10, 11, 12, 13, 14, 15,
16, 17, 18, 19, 20, 21, 22, 23, 24, 25, etc., plus a fraction such as .00, .05, .10, .15, .20, .25,
.30, .35, .40, .45, .50, .55, .60, .65, .70, .75, .80, .85, .90, or .95) or more prior; a drug or use
thereof, for which market approval was based on reference to human clinical studies (e.g.,
phase-1, safety, or human testing, e.g., phase-2, or phase 3) initiated a term (e.g., at least
about an integral number of years, like 1, 2, 3, 4, 5, 6, 7, 8, 9, 10, 11, 12, 13, 14, 15, 16, 17,
18, 19, 20, 21, 22, 23, 24, 25, etc., plus a fraction such as .00, .05, .10, .15, .20, .25, .30,
.35, .40, .45, .50, .55, .60, .65, .70, .75, .80, .85, .90, or .95) or more prior; and/or a
substitute or replacement part of an implant or device. Databases, processing systems,
procedures, and operations comprising or handling records including data from such
methods are also conceived.
Another aspect can comprise a method of gaining, or achieving, exemption from
infringement of a national patent with claims covering a patented technology, comprising
using the patented technology solely for uses reasonably related to the development or
submission of information under a Federal law which regulates the manufacture, use, offers
to sell, or sale of drugs. Methods are conceived with any and all combinations of features
described. In certain embodiments, the use reasonably related to the development or
submission of information under a Federal law which regulates the manufacture, use, offers
to sell, or sale of drugs can be a clinical trial. In some embodiments, the national patent can
be issued in the US, Europe, EP, UK, Germany, France, Spain, Italy, Sweden, Canada,
Mexico, Japan, China, South Korea, or India. In some embodiments, the trial can be
a single-site trial, or can be a virtual-site trial; and/or the trial includes at least some subjects
who are provided assurance, which can be therapeutic and/or financial. In yet some
embodiments, the trial (in all various combinations): can be funded, at least in part, by a
third-party payer, an employer, or a managed care company, or includes pay-to-participate
subjects, whose payments cover at least some drug and/or participation costs; can be
conducted by a third-party sponsor; can be sizeable, such as thousands or tens of thousands
of subjects; can be conducted for a substantial period, such as at least about 4, 6, 7, 8, 9, or
more years; can be a phase-2, a phase-3, a merged, or a phase-4 clinical trial; can provide
exemption from patent infringement, including from an “evergreening” patent; can allow
administering of drug covered by market exclusivity; and/or can be testing a drug or
equivalent which is listed in table 1, 2, or 3. In other embodiments, the national patent
includes one or more claims which is directed to: provide an “evergreening” patent
coverage, including subject matter directed to any of: a method of manufacturing; a new
method of manufacturing an existing drug (or structural similar, salt, polymorph, solvate,
biosimilar, or chemical or metabolic conversion structure, or the like); a new target patient
group for an existing drug (or structural similar, salt, polymorph, solvate, biosimilar, or
chemical or metabolic conversion structure, or the like); a new means to use or administer
an existing drug (or structural similar, salt, polymorph, solvate, biosimilar, or chemical or
metabolic conversion structure, or the like), including with use of a companion diagnostic;
a new indication for an existing drug (or structural similar, salt, polymorph, solvate,
biosimilar, or chemical or metabolic conversion structure, or the like); a combination
composition comprising an existing drug; a new use for an existing drug (or structural
similar, salt, polymorph, solvate, biosimilar, or chemical or metabolic conversion structure,
or the like) composition; a variant isomer of an existing drug (or structural similar, salt,
polymorph, solvate, biosimilar, or chemical or metabolic conversion structure, or the like);
a new formulation for an existing drug (or structural similar, salt, polymorph, solvate,
biosimilar, or chemical or metabolic conversion structure, or the like); a new route
of administration for an existing drug (or structural similar, salt, polymorph, solvate,
biosimilar, or chemical or metabolic conversion structure, or the like); a drug (or structural
similar, salt, polymorph, solvate, biosimilar, or chemical or metabolic conversion structure,
or the like) or use thereof which had been initially approved (either in US, or elsewhere) a
time (e.g., at least about an integral number of years, like 1, 2, 3, 4, 5, 6, 7, 8, 9, 10, 11, 12,
13, 14, 15, 16, 17, 18, 19, 20, 21, 22, etc., plus a fraction such as.00, .05, .10, .15, .20, .25,
.30, .35, .40, .45, .50, .55, .60, .65, .70, .75, .80, .85, .90, or .95) or more prior; a drug (or
structural similar, salt, polymorph, solvate, biosimilar, or chemical or metabolic conversion
structure, or the like) or use thereof, first sold, in the US or elsewhere, a period (e.g., at
least about an integral number of years, like 1, 2, 3, 4, 5, 6, 7, 8, 9, 10, 11, 12, 13, 14, 15,
16, 17, 18, 19, 20, 21, 22, 23, 24, 25, etc., plus a fraction such as .00, .05, .10, .15, .20, .25,
.30, .35, .40, .45, .50, .55, .60, .65, .70, .75, .80, .85, .90, or .95) or more prior; a drug or use
thereof, for which market approval was based on reference to human clinical studies (e.g.,
phase-1, safety, or human testing, e.g., phase-2, or phase 3) initiated a term (e.g., at least
about an integral number of years, like 1, 2, 3, 4, 5, 6, 7, 8, 9, 10, 11, 12, 13, 14, 15, 16, 17,
18, 19, 20, 21, 22, 23, 24, 25, etc., plus a fraction such as .00, .05, .10, .15, .20, .25, .30,
.35, .40, .45, .50, .55, .60, .65, .70, .75, .80, .85, .90, or .95) or more prior; and/or a
substitute or replacement part of an implant or device.
Also disclosed herein is a method of administering to patients a drug covered by market
exclusivity (i.e., during a period of blockage of submitting an application for market
approval), comprising using the drug in a clinical trial before submission of an application
for market approval. Methods are conceived with any and all combinations of features
described. In some embodiments, the blockage of submitting an application for market
approval exists in the US, Europe, EP, UK, Germany, France, Spain, Italy, Sweden, Canada,
Mexico, Japan, China, South Korea, or India. In some embodiments of the method, the trial
(in all various combinations): is funded, wholly or at least in part, by a third-party payer, an
employer, or a managed care company, or includes pay-to-participate subjects, whose
payments cover all or some drug and/or participation costs; is conducted by a third-party
sponsor; is sizeable, such as hundreds or thousands or tens of thousands of subjects; can
provide exemption from patent infringement, including from an “evergreening” patent;
and/or is conducted for a substantial period, such as at least about 4, 6, 7, 8, 9, or more years.
In some embodiments, the trial is a single-site trial, or is a virtual-site trial; and/or the trial
includes at least some subjects who are provided assurance, which is therapeutic and/or
financial. In certain embodiments, the trial is a phase-2, or -3, or merged clinical trial; and/or
is testing a drug or equivalent which is listed in table 1, 2, or 3. A trial period can occur
before the submission of an application for market approval, which is otherwise precluded by
certain forms of “market exclusivity”. Here, trials with other features, e.g., payer funding;
pay-to-participate subjects; third-party sponsor; size, and/or duration are also provided. As
such, clinical trials may provide both safe harbor from patent issues and avoidance of block
from “market exclusivity”.

### EXAMPLES

### Example 1—Classical Clinical Trial

The classical paradigm for a clinical trial involves development of a candidate drug, whether small molecule or biologic, by the drug developer, who may typically be a trial sponsor and a manufacturer, or its equivalent. See, e.g., Piantadosi (2017) Clinical Trials: A Methodologic Perspective (Wiley Series in Probability and Statistics) 3rd Ed. Wiley, ISBN-10: 1118959205, ISBN-13: 978-1118959206; and Friedman, et al. (2015) Fundamentals of Clinical Trials 5th ed. Springer, ISBN-10: 3319185381, ISBN-13: 978-3319185385; and an extensive library of textbooks and treatises on fundamentals of clinical and research trials in the medical sciences and particularly the pharmaceutical sciences. See also clinicaltrials.gov.

Typically, a drug candidate is subjected to extensive preclinical research studies. Then the candidate progresses to clinical research involving II, III studies. Studies are performed to understand the pharmacology, mechanism of action, safety, efficacy, and other relevant features for therapeutic use in humans. Before drug is introduced to humans, various review board approvals can be collected to ensure that proper medical ethics and proper patient protection procedures are incorporated.

The classical paradigm for the drug development process is the Phase I safety dose ranging study, the P-II safety study, and the P-III efficacy study. The development program is designed to be quick, inexpensive, and use small numbers of patients to progress the candidate to clear regulatory requirements. As such, the number of patients involved in the P-I study may be tens of patients; in P-II often <200 patients; and in P-III often 300-1,500 patients. Generally, the drug developer sponsors the trial and bears the clinical trial costs, as money invested to be recovered by later sales upon drug approval. Diagnostic evaluations are performed on the patients to determine whether there are any toxicity or adverse side effects from dosing, and to document any side effects on clinical indication being tested. Once regulatory approval for use for the intended disease indication is received, there may be no financial incentive to conduct exhaustive, additional clinical research to determine optimum drug usage, dosing, therapeutic strategy, companion theragnostic evaluation, and other features to optimize treatment outcome in patients. Most of these features fall into the area characterized as “practice of medicine”. For a majority of commercially approved drugs, approx. 5-20% of the patients achieve remission or excellent response, and as high as 30-70% of the patients can be poor or non-responders.

In the classical paradigm, drug developer, e.g., pharmaceutical company, is the innovator and has patent freedom to operate, with few or no obstructions to conduct clinical trials from interference from patents. The financing of the clinical trial and associated costs, e.g., cost of drug, trial administration costs, and so forth are paid by the drug developer with the expectation of recovery (return on investment) from profits on sales. Although there is uncertainty of ultimate market approval, the trials are statistically sufficiently powered to achieve desired endpoints and trial outcomes.

### Example 2—Classical § 271(e) Trial

For a classical generic, whether small molecule or biologic, trial, § 271(e) allows for exemption (or immunity) from patent infringement for a clinical trial. This has been used for the Phase-I, II, III clinical trials to submit data for FDA approval which allow entry into the market as soon as the corresponding blocking patents expire, e.g., which block sales of the new competitors.

### Example 3—Generic/Biosimilar Approval

A. Overseas Approvals; Trials Run Ex-US

In certain circumstances, to seek regulatory approval from FDA, data is generated ex-US. The reasons the trial is conducted overseas may be purely for administrative and logistical reasons, and may include lower costs, or otherwise, e.g., for various reasons patients are not readily accessible. In either case, the data can be submitted to FDA to seek approval for sale in the US. In some situations, data may be assembled, but the submission with FDA is barred if the drug is still patent protected.

B. Approved by FDA but Sales Blocked by Patents

In some situations, drug safety might be established by a trial performed in the US, but sales of the drug may be prohibited, e.g., by blocking patents that prohibit sales in the US. In some situations, pharmacological equivalence might be established or where such equivalence might be recognized by medical or regulatory authorities outside the US, but there may be other reasons, e.g., patents, which block sales of the drug in the US.

### Example 4—Pay-to-Participate Clinical Trials

A. Clinical Trial—Conventional Paradigm

In classical or conventional clinical trials, trial participants (patients) are either paid to participate, or otherwise drug is supplied by the sponsor, e.g., the drug developer. Because of some perceived uncertainty or risk in administering a new drug, there has been some question as to whether paying trial participants to participate is ethical, and one factor in that evaluation is how much the trial participants can be paid. In an alternative scenario, whether volunteers might pay to participate in Phase-I, II, III trials, i.e., to access, e.g., a clinical drug candidate that is not yet approved for commercialization. Only recently has the concept of “pay-to-participate” trials been tried. These have been used in stem cell trials and some neurological medications in smaller trial sizes. These small numbers were, e.g., 50, 70, 90, 110, 130, 150, 175, 200, 225, 250, 300, or similar “limited” numbers of pay-to-participate patients in prior art studies. As such, the use of such a clinical trial funding model for larger than “limited” or “small numbers” of such pay-to-participate subjects, is a new strategy described herein, and might be described as “sizeable” pay-to-participate trials, to distinguish from the smaller ones. “Sizeable” trials would be those comprising larger, e.g., N=100,000. Third-party payers cover drug and/or participation costs, either wholly or in significant part.

B. Unapproved by FDA; Biosimilar or Generic

In some situations, drug safety might be established by a combination of trial and equivalence to an approved drug, but the drug has not yet been approved as a biosimilar or generic equivalent. In such a situation, e.g., phase-3 trial, the biosimilar candidate is evaluated to establish efficacy. Under the pay-to-participate trial, patients access the drug; third-party payer and (or) patients may cover drug and/or participation costs. In certain cases, efficacy and/or financial assurance is additionally provided as part of the trial feature.

C. Approved Drug

In Phase-4 trials, in the area of generics, e.g., small molecule or biologics, which are molecular versions which have been tested and/or shown to be both structurally and functionally equivalent or identical to already established therapeutics, the therapeutic differences may be undetected or undetectable. Certain trials are run to determine whether any differences, if any, are actually detectable among the generics of a drug and its biosimilars. Pay-to-participate may originate from third-party payers, who may be insurers, employers, and the like. As above, in appropriate situations, efficacy and/or financial assurance is additionally provided.

D. Innovator Desires Access to Alternative Trial Financing: Non-271(e) Clinical Trials

A non-271(e) clinical trial may be conducted which is not impacted by the 271(e) exemption. For example, an innovator, who is a small to mid-sized biotechnology company, who holds patents to the test drug and has freedom to operate, wants to access the pay-to-participate feature. For instance, the drug is approved by FDA based on Phase-2 clinical trial data through fast-track process or breakthrough therapy designation. As mandated by the FDA, post approval, the innovator has to perform one Phase-3 clinical trial, which may also be referred to as a post-approval trial equivalent to that of a Phase-4 clinical trial, or two separate Phase-3 clinical trials, followed by a Phase-4 trial. Under such a scenario, during the non-271(e) trial, patients pay, directly or indirectly, for access to the drug. Thus, instead of having only a typical large pharmaceutical company partner to fund the trial process, e.g., Phase-3 trial (both drug and treatment), the innovator can access an alternative option to fund the trial and simultaneously derive revenue during the trial. In one instance, the innovator has an option to pay for the clinical trials, e.g., about 5%, 10%, 25%, etc. of the trial costs. In another instance, along with the third-party managed care company, the innovator has an option to co-participate or co-lead in performing the clinical trials. In this instance, the innovator has partial or full access to the trial data. Having a different partner, e.g., a third-party managed care company instead of a large pharmaceutical company, the innovator can have an autonomous control over how the trial is performed as opposed to, e.g., short-term endpoints and faster trial completion presumably imposed by a large pharmaceutical company.

In one instance, a third-party payer can pay for the trial costs, which may include payments for drug to innovator. In another instance, a third-party sponsor, e.g., a managed care company, conducts the trial.

### Example 5: Patent Protected Drug, Highly Effective Early Results

A phase-2 clinical trial demonstrates highly effective safety and efficacy results, e.g., 85-100% cure rate. The drug has not yet completed a phase-3 trial; however, the regulatory agency, e.g., the FDA is not willing to approve without a phase-3 study, which may take ca. five more years to complete. A third-party payer, e.g., private payer, agrees to conduct a pay-to-participate phase-3 clinical trial through a third-party sponsor, e.g., a managed care company, to further demonstrate the efficacy of the drug. FDA expedites the drug approval based on the phase-2 trial data but requires that the innovator pharmaceutical company conduct a phase-3 trial (e.g., N=400-600, and in rare diseases, e.g., N=60-150). Such a phase-3 clinical trial effectively becomes a phase-4 trial. Because of the funding constraints, the innovator turns to a third-party managed care company to conduct this phase-3 trial, which is a non-271(e) clinical trial. FDA and CMS (Centers for Medicare and Medicaid) agree for such a third-party sponsored trial. The managed care company conducts a phase-3, pay-to-participate clinical trial. While the objectives of the phase-3 trial are met, patients are also able to access the drug because of this trial. This approach is herein referred to as merging of trials. In such a non-271(e) clinical trial setting, the innovator, e.g., a mid-sized biotechnology company, has an option to participate in the trial, along with the third-party managed care company; the innovator has an option to pay for the clinical trial costs, e.g., about 5%, 10%, 30%, 40%, etc., of the costs. Such Phase-3 or Phase-4 clinical trials are of conventional trial sizes, e.g., N=300-1000, and the trial duration, e.g., 2-5 years. Subsequently, a sizeable Phase-4 clinical trial is conducted.

By virtue of the access to pay-to-participate component funding some or all of drug cost, the drug manufacturer agrees for such a third-party funded, third-party sponsored clinical trial.

### Example 6: Platform Clinical Trial Overview

A clinical trial is designed to investigate a drug biosimilar in a Phase IV clinical trial. The clinical trial might not be a placebo controlled, double blind, subject randomized clinical trial. In the clinical trial, the biosimilar is obtained directly from a generic drug manufacturer. The clinical trial is, in certain embodiments, carried out in a facility with a drug formulary, a pharmacy, a laboratory, and a disease and therapy management entity onsite. The clinical trial is, in various embodiments, managed through a virtual (i.e., telehealth or site-less) clinical platform as exemplified in FIGS. 2 and 3. Alternatively, a single-site clinical trial is performed, but with uniform theragnostic criteria and evaluation for a larger trial, e.g., sizeable trial.

The clinical trial is, in certain cases, carried out for at least 2 years. In certain embodiments, biological samples from patients are collected and archived to allow for certain retrospective analyses.

### Example 7: Enrollment of a Subject

A subject in need of treatment is enrolled in a clinical trial. The subject's prescription drug coverage amount is used to pay for the drug cost, while the subject's employer pension plan may itself pay for the trial. The trial is conducted by a third-party clinical trial sponsor. The subject pays, e.g., a 10% of the drug cost to participate in the study.

### Example 8—Treatment in a Clinical Trial

FIG. 1 shows, in a simplified depiction, the healthcare operation described in the earlier application, with an example of focus on RA. The treatment side includes the possibility, as described herein, of treating with drugs approved for sale but having issues with selling because of patent issues. FIG. 2 shows how the operation of a clinical trial platform is seamlessly integrated into the infrastructure within the described business. The platform often is a virtual, (i.e., site-less) clinical platform devoid of physical clinical sites, or may have one or more “trial sites”. The platform embodiment further described in FIG. 3, has components, preferably integrated together, of a drug formulary, a financial arm which deals with the insurance and payment functions including prior authorization needs to deliver drug for treatment, a Disease and Therapy Management Care team, and theragnostic laboratories. Most of the clinical trial functions including disease and therapy management (DTM), patient therapeutic adherence (PTA) and therapy guideline adherence (TGA) by specialist physicians involved in clinical trials are managed, all or in part, through the virtual (telehealth) clinical platform. These have been described in the Healthcare application. These platforms work together to provide treatment to patients, but provides the option in treatment to use drugs covered by patents, particularly where use would otherwise be blocked outside of a clinical trial context.

The Disease and Therapy Management Team often is a telehealth group, which may be physically disperse but have collected together necessary expertise of multiple specialists for complex disease conditions, e.g., rheumatoid arthritis, multiple sclerosis, lupus nephritis, Crohn's disease, hematological malignancies such as B-NHL, CLL. Consultations are regular or frequent, as necessary, at a very high level with access (preferably in real time) to medical records and can prescribe and make available the appropriate treatments to the patient where the patient is. Thus, the site of treatment may be different from the physical location of the prescribing or clinical trial physician.

### Example 9—Phase-IV Clinical Trials

Rheumatoid Arthritis:

Drugs under investigation: e.g., etanercept, adalimumab, infliximab, rituximab, tocilizumab, tofacitinib, sarilumab, canakinumab

Either innovator drug or generic drugs are investigated in this trial

Objectives of the Trial:

Subset(s) and subtype(s) of patients achieving clinical remission, excellent response, non-responders to these medicaments.

Time to remission, time to disease relapse after remission period, selection of therapies after disease relapse (same drug versus different drug) to achieve clinical remission. Evaluate longer term responsiveness to treatment.

Approach (Some or all; Controlled Comparisons):

Patients are stratified according to disease subtypes, disease severity per pathophysiology, and/or treatment response per mechanism of action (MOA) of the drugs. Theragnostic evaluation can test MOA effectiveness as early readout.

Subset(s) of patients achieving remission, and continued remission, e.g., for 2-4 years.

Subset(s) of patients where disease relapses, and their response patterns to re-treatment of the same drug or an alternate drug, and determination of mechanistic rationale for this (See FIG. 4).

Methods of achieving clinical remission in chronic autoimmune diseases, e.g., RA, for 6-10+ years.

Patients achieving clinical remission to maintenance treatments (e.g., rituximab maintenance treatment every 3-6 months).

Patients achieving clinical remission to monotherapy treatments (i.e., adalimumab monotherapy and methotrexate withheld).

Methods of Stratification (Some or all; can Compare Different Theragnostic Modalities):

Subset(s) of patients stratified based on disease severity, correlated to treatment response to these drugs.

Subtypes of RA patients based on immunology—IgG RF^{+/-}; IgG ACPA^{+/-}; IgA RF^{+/-}; IgA ACPA^{+/-}; citrullinated Fibrinogen (cFib^{+/-}); CRP^{Hi/Lo}. Both qualitative and quantitative measurements are correlated. E.g., Presence of high levels of either IgG RF or IgG ACPA in patient group G-1 (see 3 below) can lead to severe RA.

Determination of FcGR-3A, 2A polymorphisms, FcAR (CD89; Ser/Gly248 polymorphism), FcGR-3B polymorphisms; patients stratified into 3×3 matrices to determine disease severity (3Ax2A; 3Bx2A; FcARx2A). E.g., Group-1 in the 3×3 matrix is characterized by FcGR-3A (VV¹⁵⁸) and FcGR-2A (HH¹³¹) polymorphisms, and Group-9 by FF¹⁵⁸ and RR¹³¹ polymorphisms. G-1 and G-9 are the excellent and poor responder subsets, respectively, to ADCC-mediated single-course antibody (e.g., rituximab) monotherapy.

Patients eligible for maintenance therapy (e.g., rituximab) based on ADCC functional assay, including minimal residual disease (MRD) flow cytometry, depletion and re-population of B-cell sub sets.

Inflammatory Cytokine/Immune Cells Evaluations:

Determination of cytokine levels, e.g., TNF-alpha, IL-1, IL-1β, IL-6, and the like. correlated to FcGR, FcAR polymorphisms (see steps-2,3 above). Drugs are not as effective if these cytokine levels do not decrease upon treatment. Depletion of immune effector cells (platelets, NK-cells, inflammatory macrophages) leads to decreased cytokine levels.

Determination of B-cell subsets (CD19⁺ naïve; CD27⁺ memory, CD38⁺ plasma) by minimal residual disease (MRD) flow cytometry: complete versus partial depletion, re-population of these cells correlate to treatment response. Complete depletion leads to excellent response.

Determination of FRβ⁺ (folate receptor beta) inflammatory macrophages: depletion and re-population of these cells correlate to better treatment response and disease relapse, respectively.

Determination of T-cell subsets (naïve, regulatory (Treg), and inflammation-related cells (IRC)) in ACPA⁺ early RA: higher naïve cell frequency is associated with disease remission.

Additional Phase-IV Trials

| Disease | | | |
|---|---|---|---|
| Indications | Approach | | |
| Multiple Sclerosis | Identification of responder and non- | | |
| Drugs | responder subsets to these therapies: | | |
| investigated: | a. | Severity-based stratification (FcGR- | |
| ocrelizumab, | | 3A/2A) of patients and selection of | |
| alemtuzumab, | | appropriate therapies; | |
| ofatumumab, | b. | Poor (phagocytotic) immune | |
| natalizumab, | | complex clearance is associated | |
| dimethyl | | with disease severity: very poor | |
| fumarate, | | in Groups-8, 9; | |
| daclizumab | c. | Depletion and re-population | |
| | | patterns of B-cell and T-cell subsets | |
| | | by MRD-FC | |
| SLE (Lupus), | Identification of responder and non- | | |
| lupus | responder subsets to these therapies: | | |
| nephritis | a. | Severity-based stratification (FcGR- | |
| indications | | 3A/2A) of patients and selection of | |
| Drugs | | appropriate therapies; | |
| investigated: | b. | FcGR 3A/2A based 3x3 matrix generation: | |
| belimumab, | | i. | Poor (phagocytotic) immune |
| rituximab, | | | complex clearance is associated |
| obinutuzumab, | | | with disease severity: very poor |
| voclosporin, | | | in Groups-6, 8, 9; |
| abatacept, | | ii. | Depletion and re-population |
| anifrolumab | | | patterns of B-cell and T-cell |
| | | | subsets by MRD-FC |
| | | iii. | Correlation of autoantibodies: |
| | | | e.g., ANA, anti-dsDNA, anti- |
| | | | Smith, anti-U1RNP, anti-Ro/ |
| | | | SSA and anti-La/SSB to FcGR- |
| | | | 3A/2A polymorphisms. |

| B-NHL | Identification of responder and non- | |
|---|---|---|
| Drugs | responder subsets to these therapies: | |
| investigated: | a. | FcGR 3A/2A based 3x3 matrix |
| rituximab, | | generation; |
| ibrutinib, | b. | ADCC functional assay |
| lenalidomide, | c. | Characterization of B-cell subsets |
| obinutuzumab, | | by MRD-FC to determine depletion |
| axicabtagene | | and re-population of B-cell subsets; |
| ciloleucel | | and use of this information to select |
| | | patients for single course rituximab |
| | | monotherapy and maintenance |
| | | therapy. |
| CLL | Identification of responder and non- | |
| Drugs | responder subsets to these therapies: | |
| investigated: | a. | FcGR 3A/2A based 3x3 matrix |
| rituximab, | | generation; |
| ibrutinib | b. | ADCC functional assay; |
| | c. | Characterization of B-cell subsets |
| | | by MRD-FC to determine depletion |
| | | and re-population of B-cell subsets; |
| | | and use of this information to select |
| | | patients for single course rituximab |
| | | monotherapy and maintenance therapy. |

### Example 10: Development/Tracking of Diagnostics; Cell or Tissue Based Therapeutics; Vaccines; Gene Therapy; Implants; Prostheses; and the Like

Related activities which are for uses reasonably related to the development and submission of information under a Federal law which regulates the manufacture, use, offer to sell, or sale of drugs or veterinary biological products, are performed for things other than medicaments. As described above, these may include articles recognized in the US Pharmacopoeia, or official National Formulary, or supplements thereto; articles intended for use in the diagnosis, cure, mitigation, treatment, or prevention of disease in man or other animals; articles (other than food) intended to affect the structure or any function of the body of man or other animals; and/or articles intended for use as a component of the above. Likewise, devices include an instrument, apparatus, implement, machine, contrivance, implant, in vitro reagent, or other similar or related article, including any component, part, or accessory, which is recognized in the official National Formulary, or the United States Pharmacopeia, or any supplement to them; intended for use in the diagnosis of disease or other conditions, or in the cure, mitigation, treatment, or prevention of disease, in man or other animals; or intended to affect the structure or any function of the body of man or other animals, and which does not achieve its primary intended purposes through chemical action within or on the body of man or other animals and which is not dependent upon being metabolized for the achievement of its primary intended purposes. Thus, studies or procedures related to development and/or submission of information, e.g., non-routine submission of data or results, to review aspects of these are subject to the uses described herein.

Development of various diagnostic or theragnostic testing methods, e.g., for establishing reproducibility of measure or validation of relationship to treatment outcome, are subject to these methods. Companion diagnostic methods, linked to medicament usage, which might become part of drug labels are included.

Similarly, articles related to cell- or tissue-based therapies or methods are subject to regulatory review. This can include cell transfers, transfusions, stem cell or sorted subpopulations of cell types, organ parts, cell or organ transplants, vaccines and other preventative treatments, devices, gene therapy related materials and devices, implants, prostheses, and equipment which are attached permanently, semi-permanently, or temporarily to a patient in treatment or therapy. These include critical or related compositions, supplementary compositions, articles, devices, scaffolds for cells or tissue implants, equipment for manufacture, production, or use of such, direct or indirect methods of use (including testing of what methods to avoid or which lead to unsuccessful use), and other efforts to validate or evaluate safety and/or efficiency in use.

### Example 11: Further Clinical Studies

Because the extended clinical trial is no longer constrained by certain limitations of the classical clinical trial paradigm, therapeutically relevant questions are answered in these trials, and these shall include:
a) Long term treatment outcomes such as time to remission, sustained remission period, time to relapse
b) Who are the best responders and worst responders to a treatment
c) What are optimal theragnostics; theragnostic modalities; companion theragnostics
d) How is therapy optimally administered (e.g., single course versus maintenance treatment) and monitored
e) What are the alternative treatments; secondary or further improvements
f) What are the mechanisms of action of the drugs; response mechanisms in defined disease subtype(s)
g) What are the personalized medicine complexities; biomarkers
h) What are the compliance issues such as patient therapeutic adherence, treatment guideline adherence.

### Example 12: Third-Party Clinical Trial Sponsor

From a patient pool of N=300,000 rheumatoid arthritis (RA), a third-party sponsor conducts N=200,000 patient clinical trial in the U.S. It is a 271(e) clinical trial setting. It is a single-site trial, e.g., virtual trial operated through a digital health platform. The rest of the N=100,000 RA patients undergo conventional treatment protocol. Thus, this third-party sponsor is not only a clinical trial sponsor but also a treatment provider.

The RA patient pool comes from multiple private payers, employers, pension funds, and government payers, e.g., Medicare. All these entities, collectively referred to as third-party payers, have approved and opted for such a treatment cum trial design. Patients decide to choose either the clinical trial option or the conventional treatment option. Various theragnostic procedures are used to stratify RA into distinct disease subtypes and subsets of patients.

The pharmaceutical companies that developed and/or commercialize these drugs are not involved in this trial, e.g., they are not the trial sponsors, nor do they pay for the clinical trial costs. A clinical research organization (CRO) representing pharmaceutical companies is not part of this trial. In some embodiments, the third-party sponsor is responsible for designing, managing, and/or executing the clinical trial; the trial data developed by the third-party sponsor are owned by the sponsor. In some embodiments, a sponsor is the sole representative to communicate with FDA for the clinical trial matters. In some embodiments, the financial and/or legal liabilities related to the clinical trial rest with the third-party sponsor.

It is a 10-year clinical trial that adopts adaptive trial design. Multiple drugs are tested in a single clinical trial. One of the objectives is to determine the excellent responders (subsets) and non-responders (subsets) and mechanistic correlations to each of the therapy. Another objective is to provide patient-specific N=1 assurance, e.g., both efficacy and financial assurances. Efficacy and financial assurances are specific to that subject in the trial. For example, in RA, financial assurance can refer to a limited money back guarantee equivalent to the coinsurance amount. Several biologics and small molecule drugs are employed in this trial. Some of the biologics include: etanercept, adalimumab, tocilizumab, infliximab. A small molecule includes tofacitinib. Corresponding biosimilars, e.g., adalimumab biosimilar, and small molecule generic drugs are also included in this trial.

To conduct this clinical trial, the trial sponsor buys these drugs either directly from pharmaceutical companies or from distribution networks. The trial sponsor uses either net price or list price for pricing calculations to pay for the drugs.

Clinical trial data and the patient data are maintained in clinical systems and data warehouse, and all such data, e.g., patient-specific, disease-specific, and population-specific, developed by the sponsor are proprietary to the third-party clinical trial sponsor. Data generated from the conventional treatment protocol are maintained in data warehouse. Provided that the sponsor authenticates the access, the data can be reviewed, analyzed, and used by payers, providers, physicians, pharmacies, diagnostic companies, or pharmaceutical companies.

### Example 13: Systems, Data Development and Usage

Data can exist in the form of medical records, which are stored in various ways including electronically. Any person or entity who creates (clinical) data for entry to record, facilitates such (e.g., helps subject in the process of seeing, organizing to see doctors, healthcare professionals, receptionists, sample collection, sample evaluation (diagnostic technicians), data interpretation, record entry, use of data (specifically payer, insurer, actuarial), planning of study (and subjects) are included. This includes subjects directly, or anyone who arranges for subjects, collects samples, evaluates samples, interprets data, uses data either directly or indirectly. This specifically includes insurers who use data (prior authorization, payment of claims, subset patients, determine subsetting, actuarial categories, and the like); what sponsors do (e.g., plan study, determine who to include, what to study, where to do study, when to start/end study and endpoints, why study is performed (objective), how study strategy and design is done, and the like), what study performers do (e.g., get subject informed consent, interact with subject, identify and arrange to evaluate and treat subject, collect samples when appropriate, arrange for sample evaluation and data return, use data in evaluation of subject/treatment, and the like), what laboratory technicians do (e.g., collect sample, evaluate controls, evaluate test samples, archive samples, record and report data back to record, and the like), and whoever else have access to the subjects, samples, or data (clinical data as opposed to identification data).

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

1-135. (canceled)

136. A clinical trial testing a drug, wherein said trial can be funded at least in part by a third-party payer, which may be a healthcare payer or insurer, and wherein said trial may not be substantially funded by a pharmaceutical company that developed and/or commercialized said drug.

137. The trial of claim 136, wherein said trial comprising a sizeable number of subjects, wherein said subjects may pay to participate in the trial.

138. The trial of claim 137, wherein said trial:
a) can be of substantial size, wherein at least a sizeable number of said subjects may directly pay-to-participate, or indirectly pay-to-participate, e.g., through an insurer or other payer;
b) can be a single-site trial, or can be a virtual-site trial; and/or
c) can include at least some subjects who are provided assurance, which may be a therapeutic and/or financial assurance.

139. The trial of claim 137, wherein said trial:
a) can be funded, wholly or at least in part, by a third-party payer, such as a private payer, government payer, pension fund, employer, or managed care company, but may not be substantially funded by a pharmaceutical company that developed and/or commercialized said drug;
b) can be large, such as thousands or tens of thousands of subjects;
c) can be conducted for a substantial period, such as at least about 4, 6, 7, 8, 9, or more years;
d) can be a phase-4 clinical trial; and/or
e) can be for testing a drug or equivalent listed in table 1, 2, or 3.

140. A method of conducting a clinical trial of substantial size, wherein at least a sizeable number of subjects may enroll on a pay-to-participate basis.

141. The method of claim 140, wherein said sizeable number of subjects may pay-to-participate either directly or their cost can be borne wholly or at least in part by a third-party payer.

142. The method of claim 140, wherein said trial:
a) can be a single-site trial, or can be a virtual-site trial;
b) can include at least some subjects who are provided assurance, which can be a therapeutic and/or financial assurance;
c) can be conducted, wholly or at least in part, by a third-party sponsor, such as a managed care company or a fully-integrated payer having managed care capabilities, but may not be funded by a pharmaceutical company that developed and/or commercialized the drug that is being tested;
d) can be large, such as thousands or tens of thousands of subjects;
e) can be conducted for a substantial period, such as at least about 4, 6, 7, 8, 9, or more years;
f) can be a phase-4 clinical trial; and/or
g) can be for testing a drug or equivalent which is listed in table 1, 2, or 3.

143. A clinical trial testing a drug, wherein said trial can be conducted by a third-party sponsor, and said third-party sponsor cannot be a pharmaceutical company that developed, and/or commercialized said drug.

144. The trial of claim 143, wherein said trial:
a) conducted by a third-party sponsor can be a managed care company;
b) can be a single-site trial, or can be a virtual-site trial; or
c) can include at least some subjects who are provided assurance, which can be a therapeutic and/or financial assurance.

145. The trial of claim 144, wherein said trial:
a) can be funded, wholly or at least in part, by a payer, an employer, or managed care company, or wholly or in part by pay-to-participate subjects, whose payments can cover full or partial drug and/or participation costs;
b) can be sizeable, such as thousands or tens of thousands of subjects;
c) can be conducted for a substantial period, such as at least about 4, 6, 7, 8, 9, or more years;
d) can be a phase-4 clinical trial; and/or
e) can be for testing a drug or equivalent which is listed in table 1, 2, or 3.

146. A method of conducting a clinical trial by a third-party sponsor.

147. The method of claim 146, wherein said trial:
a) conducted by a third-party sponsor can be a managed care company, or a healthcare payer having managed care capabilities;
b) said trial can be a single-site trial, or can be a virtual-site trial; and/or
c) said trial can include at least some subjects who are provided assurance, which can be a therapeutic and/or financial assurance.

148. The method of claim 146, wherein said trial:
a) either can be funded, wholly or at least in part, by pay-to-participate subjects whose payments can cover full or partial drug and/or participation costs; or may be paid, wholly or at least in part, by a payer such as an insurer, employer, or a managed care company;
b) can be sizeable, such as thousands or tens of thousands of subjects;
c) can be conducted for a substantial period, such as at least about 4, 6, 7, 8, 9, or more years;
d) can be a phase-4 clinical trial; and/or
e) can be for testing a drug or equivalent which is listed in table 1, 2, or 3.

149. A method of gaining exemption from infringement of a national patent with claims covering a patented technology, which can comprise using a patented technology solely for uses reasonably related to the development or submission of information under a Federal law which regulates the manufacture, use, offers to sell, or sale of drugs.

150. The method of claim 149, wherein the uses reasonably related to the development or submission of information under a Federal law which regulates the manufacture, use, offers to sell, or sale of drugs can be a clinical trial.

151. The method of claim 149, wherein said national patent can be issued in the US, Europe, UK, Germany, France, Spain, Italy, Sweden, Canada, Mexico, Japan, China, South Korea, or India.

152. The method of claim 150, wherein said trial:
a) can be a single-site trial, or can be a virtual-site trial;
b) can include at least some subjects who are provided assurance, which can be a therapeutic and/or financial assurance;
c) can be funded, wholly or at least in part, by a third-party payer, an employer, or a managed care company; or can include pay-to-participate subjects whose payments can cover full or partial drug and/or participation costs;
d) can be conducted by a third-party sponsor;
e) can be sizeable, such as thousands or tens of thousands of subjects;
f) can be conducted for a substantial period, such as at least about 4, 6, 7, 8, 9, or more years;
g) can be a phase-4 clinical trial; and/or
h) can be for testing a drug or equivalent which is listed in table 1, 2, or 3.

153. A method of administering a drug to patients in a clinical trial, wherein said drug is covered by market exclusivity.

154. The method of claim 153, wherein the market exclusivity exists in the US, Europe, EP, UK, Germany, France, Spain, Italy, Sweden, Canada, Mexico, Japan, China, South Korea, or India.

155. The method of claim 153, wherein said trial:
a) is funded, wholly or at least in part, by a third-party payer, an employer, or a managed care company; or includes pay-to-participate subjects whose payments cover full or partial drug and/or participation costs;
b) is conducted by a third-party sponsor;
c) is sizeable, such as hundreds or thousands or tens of thousands of subjects;
d) is conducted for a substantial period, such as at least about 4, 6, 7, 8, 9, or more years;
e) is a single-site trial, or is a virtual-site trial;
f) includes at least some subjects who are provided assurance, which is a therapeutic and/or financial assurance;
g) is a phase-2 or -3 clinical trial; and/or
h) is testing a drug or equivalent which is listed in table 1, 2, or 3.


SEQUENCE LISTING (25 sequences)

SEQ ID NO: 1 - protein, 65 aa, Artificial Sequence
  Description of Artificial Sequence Synthetic polypeptide
LTYTDCTESGQNLCLCEGSNVCGQGNKCILGSDGEKNQCVTGEGTPKPQSHNDGDFEEIPEEYLQ

SEQ ID NO: 2 - protein, 20 aa, Artificial Sequence
  Description of Artificial Sequence Synthetic peptide
FPRPGGGGNGDFEEIPEEYL

SEQ ID NO: 3 - protein, 355 aa, Artificial Sequence
  Description of Artificial Sequence Synthetic polypeptide
SYQGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 4 - protein, 527 aa, Artificial Sequence
  Description of Artificial Sequence Synthetic polypeptide
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 5 - protein, 527 aa, Artificial Sequence
  Description of Artificial Sequence Synthetic polypeptide
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGNWSTAESGAECTNWQSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAAAAASPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 6 - protein, 260 aa, Artificial Sequence
  Description of Artificial Sequence Synthetic polypeptide
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 7 - protein, 497 aa, Artificial Sequence
  Description of Artificial Sequence Synthetic polypeptide
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 8 - protein, 506 aa, Artificial Sequence
  Description of Artificial Sequence Synthetic polypeptide
EFARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQDLLVDTM

SEQ ID NO: 9 - protein, 327 aa, Dickeya chrysanthemi
ADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 10 - protein, 393 aa, Artificial Sequence
  Description of Artificial Sequence Synthetic polypeptide
ALAQKRDNVLFQAATDEQPAVIKTLEKLVNIETGTGDAEGIAAAGNFLEAELKNLGFTVTRSKSAGLVVGDNIVGKIKGRGGKNLLLMSHMDTVYLKGILAKAPFRVEGDKAYGPGIADDKGGNAVILHTLKLLKEYGVRDYGTITVLFNTDEEKGSFGSRDLIQEEAKLADYVLSFEPTSAGDEKLSLGTSGIAYVQVNITGKASHAGAAPELGVNALVEASDLVLRTMNIDDKAKNLRFNWTIAKAGNVSNIIPASATLNADVRYARNEDFDAAMKTLEERAQQKKLPEADVKVIVTRGRPAFNAGEGGKKLVDKAVAYYKEAGGTLGVEERTGGGTDAAYAALSGKPVIESLGLPGFGYHSDKAEYVDISAIPRRLYMAARLIMDLGAGK

SEQ ID NO: 11 - protein, 726 aa, Artificial Sequence
  Description of Artificial Sequence Synthetic polypeptide
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIDDDDDDDDDD

SEQ ID NO: 12 - protein, 496 aa, Artificial Sequence
  Description of Artificial Sequence Synthetic polypeptide
APQPPNILLLLMDDMGWGDLGVYGEPSRETPLCSPSRAALLTGRLPIRNGFYTTNAHARNLLKKAGYVSKIVGKWHLGHRPQFHPLKHGFNIPVYRDWEMVGRYYEEFPINLKTGEANLTFLYWAVDATHAPVYASKPFLGTSQRGRYGDVADNTFVFFTSDNGAALISAPEQGGSNGPFPGHVTAGQVSHQLGSIMDLFTTSLALAGLTLMDRPIFYYRGDTLMAATLGQHKAHFWTWTVTTHNLEDHTKLPLIFHLGRDPGERFPLSFEALVPAQPQLNVCNWAVMNWAPPGCEKLGKPNLDRMAAEGLLFPNFYSANAYTPQEIVGGIPDSEQLLPEDEWFGSPNCHFGPYDNKARPQIYLQEALDFIKRQARHHPFAVREIDDSIGKILELLQDLHLCGKQTTFEGGMREPALAWWPPSDRAIDGLNLLPTLLQGRNSWENFRQGIDFCPGQNVSGASAEYQEALSRITSVVQQHQCLTPPESIPKKCLWSH

SEQ ID NO: 13 - protein, 378 aa, Artificial Sequence
  Description of Artificial Sequence Synthetic polypeptide
SGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 14 - protein, 513 aa, Artificial Sequence
  Description of Artificial Sequence Synthetic polypeptide
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 15 - protein, 298 aa, Artificial Sequence
  Description of Artificial Sequence Synthetic polypeptide
TYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 16 - protein, 449 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 521 aa, Artificial Sequence
  Description of Artificial Sequence Synthetic polypeptide
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 18 - protein, 134 aa, Artificial Sequence
  Description of Artificial Sequence Synthetic polypeptide
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 19 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 448 aa, Artificial Sequence
  Description of Artificial Sequence Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 431 aa, Artificial Sequence
  Description of Artificial Sequence Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 504 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 24 - protein, 449 aa, Artificial Sequence
  Description of Artificial Sequence Synthetic polypeptide
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 448 aa, Artificial Sequence
  Description of Artificial Sequence Synthetic polypeptide
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK